(19) United States
(12) Patent Application Publication
(10) Pub. No.: US 2021/0047410 A1
(43) Pub. Date: Feb. 18, 2021

(54) METHODS OF SELECTING AND DESIGNING SAFER AND MORE EFFECTIVE ANTI-CTLA-4 ANTIBODIES FOR CANCER THERAPY

(71) Applicants: oncolmmune, Inc., Rockville, MD (US); Children's Research Institute, Children's National Medical Center, Washington, DC (US)

(72) Inventors: Yang Liu, Baltimore, MD (US); Pan Zheng, Baltimore, MD (US); Fei Tang, Baltimore, MD (US); Mingyue Liu, Baltimore, MD (US); Martin Devenport, Gaithersburg, MD (US); Xuexiang Du, Baltimore, MD (US); Yan Zhang, Rockville, MD (US)

(73) Assignees: Oncolmmune, Inc., Rockville, MD (US); Children's Research Institute, Children's National Medical Center, Washington, DC (US)

(21) Appl. No.: 16/967,065

(22) PCT Filed: Jan. 29, 2019
(86) PCT No.: PCT/US19/15664

§ 371 Date: Aug. 3, 2020

Related U.S. Application Data
(60) Provisional application No. 62/625,662, filed on Feb. 2, 2018.
(60) Provisional application No. 62/647,123, filed on Mar. 23, 2018.

(51) Int. Cl. C07K 16/28; A61P 35/00; C12N 5/071; C12N 5/0783; G01N 33/58

(52) U.S. Cl. CPC C07K 16/2818; C07K 16/2827; G01N 33/582; C12N 5/0682; C12N 5/0637; A61P 35/00

(57) ABSTRACT

The present invention relates to compositions of anti-CTLA-4 antibodies that bind to the human CTLA4 molecule and their use in cancer immunotherapy and for the reduction of autoimmune side effects compared to other immunotherapeutic agents.

### STATEMENT REGARDING FEDERALLY SPONSORED RESEARCH OR DEVELOPMENT

This invention was made in part with Government support under Grant Nos. AI64350, CA171972 and AG036690, awarded by the National Institutes of Health. The Government has certain rights in this invention.

### FIELD OF THE INVENTION

The present invention relates to anti-cytotoxic T lymphocyte-associated antigen-4 (CTLA-4) antibodies and antigen-binding fragments thereof.

### BACKGROUND OF THE INVENTION

The classic checkpoint blockade hypothesis states that cancer immunity is restrained by two distinct checkpoints: the first is the interaction between CTLA-4 and B7 that limits priming of naïve T cells in the lymphoid organ, while the second is the Programmed Death 1 (PD-1)/B7H1(PDL1) interaction that results in exhaustion of effector T cells within the tumor microenvironment [1]. Since then, several new targets have been under evaluation in clinical trials [2] and multiple mechanisms have been described for the targeting reagents [3]. Anti-CTLA-4 monoclonal antibodies (mAbs) induce cancer rejection in mice [4-6] and patients [7-8].

Recently, a number of additional mechanisms were proposed to explain the immunotherapeutic effect of anti-CTLA-4 mAbs, including depletion of regulatory T cells (Treg) in tumor microenvironment [9-11], and blocking of transendocytosis of B7 on dendritic cells [12-13]. However, it remains to be tested whether anti-CTLA-4 antibodies induce tumor rejection by mechanisms postulated by the checkpoint blockade hypothesis, namely blocking B7-CTLA-4 interaction and functioning in the lymphoid organs to promote activation of naïve T cells [1].

The systemic effect of anti-CTLA-4 mAbs was questioned by reports proposing that the tumor immunotherapeutic effect of anti-mouse CTLA-4 mAbs depends on their interaction with activating receptor for Fc and that the therapeutic effect correlates with selective depletion of Tregs in the tumor microenvironment [9-11]. While these studies cast doubt on the dogma that anti-CTLA-4 antibodies execute their therapeutic effect at lymphoid organs, it does not address the core issue as to whether blocking the B7-CTLA-4 interaction is required for or contributes to cancer therapeutic effect, or is involved in the depletion of Tregs in the tumor microenvironment.

Despite the generally accepted concept that anti-mouse CTLA-4 mAbs induce tumor rejection by blocking negative signaling from B7-CTLA-4 interaction, the blocking activity of these antibodies [4-6, 9-11] has not been critically evaluated. On the other hand, it has been reported that the first clinically used anti-CTLA-4 mAb, Ipilimumab, can block the B7-CTLA-4 interaction if soluble B7-1 and B7-2 are used to interact with immobilized CTLA-4 [14]. However, since B7-1 and B7-2 are membrane-associated costimulatory molecules, it is unclear whether the antibody blocks B7-CTLA-4 interaction under physiologically relevant conditions.

A combination of the anti-PD-1 mAb Nivolumab and the anti-CTLA-4 mAb, Ipilimumab, significantly increased objective response rates of advanced melanoma patients [6, 7]. Promising results also emerged from this combination therapy in advanced non-small cell lung carcinoma (NSCLC) [8]. Similar clinical benefits were observed when another anti-CTLA-4 mAb (Tremelimumab) was combined with Durvalumab, an anti-PD-L1 mAb [9]. Severe adverse events (SAEs) present a major obstacle to broader clinical use of anti-CTLA-4 mAbs, either alone or in combination [6, 7]. The SAEs observed in the Ipilimumab trials led to the concept of immunotherapy-related adverse events (irAE) [10]. In particular, in combination therapy with Ipilimumab and Nivolumab (anti-PD-1), more than 50% patients developed grade 3 and grade 4 SAE. In NSCLC, Ipilimumab and Nivolumab combination therapy resulted in high response rates, although the grade 3 and 4 SAEs also occurred at high rates [8]. Likewise, the combination of Durvalumab (anti-PD-L1) and Tremelimumab (anti-CTLA-4) showed clinical activities in NSCLC [9], although this activity was not substantiated in a phase III clinical trial. High rates of grade 3 and 4 SAEs were reported and patient drop-off rates were high, presumably due to unacceptable toxicity [9]. Since a higher dose of anti-CTLA-4 mAb is associated with better clinical outcomes in both monotherapy and combination therapy, irAE not only prevents many patients from continuing on immunotherapy, but also limits the efficacy of the cancer immunotherapy effect (CITE). Furthermore, the high numbers of patient who dropped off with both anti-CTLA-4 mAbs likely attributed to the failure to meet clinical endpoints in several clinical trials [11, 12].

More recently, a head-to-head comparison of the anti-PD-1 mAb, Nivolumab, and the anti-CTLA-4 mAb, Ipilimumab, as adjuvant therapy for resected stage III and IV melanoma showed that Ipilimumab had lower CITE but higher irAE [13], further dimming the prospect of CTLA-4-targeting immunotherapy. However, Ipilimumab-treated patients who survived for three years showed no further decline in survival rate over a ten-year period [14]. The remarkably sustained response highlights the exceptional benefit of targeting CTLA-4 for immunotherapy, especially if irAE can be brought under control.

A fundamental question for the generation of safe and effective anti-CTLA-4 mAbs is whether CITE and irAE are intrinsically linked. Since genetic inactivation of CTLA-4 expression leads to autoimmune diseases in mouse and human, it is assumed that the irAE would be a necessary price for CITE. On the other hand, recent studies suggest that rather than blocking B7-CTLA-4 interaction, the therapeutic effect of anti-mouse CTLA-4 mAbs requires antibody-mediated depletion of Treg specifically within tumor microenvironment [16-18]. These studies raise the intriguing possibility that CITE can be achieved without irAE if one can achieve local Treg depletion without mimicking genetic inactivation of CTLA-4 expression. In order to test this hypothesis, it is essential to establish a model that faithfully recapitulates clinically observed irAE.

Commonly reported irAEs in patients that receive either anti-CTLA-4 or anti-CTLA-4 plus anti-PD-1/PD-L1 agents include hematological abnormalities such as pure red cell aplasia [19, 20], and non-infection-related inflammatory damage to solid organs, such as colitis, dermatitis, pneumonitis, hepatitis, and myocarditis [21-23]. While the term irAE implies an intrinsic link between CITE and autoimmune AE, there are very few investigational studies that substantiate such a link. In contrast, the inventors' previous work involving human Ctla4 knockin mice showed that the levels of anti-DNA antibodies and cancer rejection parameters do not always correlate with each other [24]. In particular, it was found that one of the antibodies tested, L3D10, conferred strongest CITE but yet induced the lowest levels of anti-DNA antibodies among several mAbs tested. Nevertheless, since the anti-CTLA-4 mAb induced adverse events are relatively mild in the mice, this model failed to recapitulate clinical observations. As such, it is of limited value in understanding the pathogenesis of irAE and in identification of safe and effective anti-CTLA-4 mAbs. Moreover, since these studies were performed before clinically used anti-CTLA-4 mAbs were available, it is unclear whether the principles were relevant to irAE induced by clinical products.

### SUMMARY OF THE INVENTION

It is assumed that anti-CTLA-4 antibodies cause tumor rejection by blocking negative signaling from the B7-CTLA-4 interactions. As disclosed herein, human CTLA4 gene knockin mice as well as human hematopoietic stem cell reconstituted mice were used to systematically evaluate whether blocking the B7-CTLA-4 interaction under physiologically relevant conditions is required for the CITE of anti-human CTLA-4 mAbs. Surprisingly, at concentrations considerably higher than plasma levels achieved by clinically effective dosing, the anti-CTLA-4 antibody Ipilimumab blocks neither B7 transendocytosis by CTLA-4 nor CTLA-4 binding to immobilized or cell-associated B7. Consequently, Ipilimumab does not increase B7 levels on DC from either CTLA4 gene humanized mice (Ctla4^{b/h}) or human CD34+ stem cell-reconstituted NSG™ mice. In Ctla4h/m mice expressing both human and mouse CTLA4 genes, anti-CTLA-4 antibodies that bind to human but not mouse CTLA-4 efficiently induce Fc receptor-dependent Treg depletion and tumor rejection. The blocking antibody L3D10 is comparable to the non-blocking Ipilimumab in causing tumor rejection. Remarkably, L3D10 progenies that lost blocking activity during humanization remain fully competent in Treg depletion and tumor rejection. Anti-B7 antibodies that effectively blocked CD4 T cell activation and de novo CD8 T cell priming in lymphoid organ do not negatively affect the immunotherapeutic effect of Ipilimumab. Thus, the clinically effective anti-CTLA-4 mAb, Ipilimumab, causes tumor rejection by mechanisms that are independent of checkpoint blockade but dependent on host Fc receptors. The data presented herein call for a reappraisal of the CTLA-4 checkpoint blockade hypothesis and provide new insights for next generation of safe and effective anti-CTLA-4 mAbs.

In addition to conferring the cancer immunotherapeutic effect (CITE), anti-CTLA-4 monoclonal antibodies (mAbs) cause severe immunotherapy-related adverse events (irAE). Targeting CTLA-4 has shown remarkable long-term benefit and thus remains a valuable tool for cancer immunotherapy if the irAE can be brought under control. An animal model that recapitulates clinical irAE and CITE would be a valuable for developing safer CTLA-4 targeting reagents. In developing a mouse model of irAE, the inventors considered three factors. First, since combination therapy with anti-PD-1 and anti-CTLA-4 is being rapidly expanded into multiple indications, a model that recapitulates the combination therapy would be of great significance for the field. Second, the fact that combination therapy results in SAEs (grades 3 and 4 organ toxicity) in more than 50% of the subjects will make it easier to recapitulate irAE in the mouse model. Third, since the mouse is generally more resistant to irAE, one must search for conditions under which the irAE can be faithfully recapitulated. As the autoimmune phenotype in Ctla4^{-/-} mice appears strongest at a young age [25, 26], and targeted mutation of the Ctla4 gene in adult mice leads to a less severe autoimmune diseases [27], the inventors had the insight that mice may be most susceptible to anti-CTLA-4 mAbs if they are administrated at the young age. Taking these factors into consideration, the inventors have identified a model system that faithfully recapitulates the irAEs observed in clinical trials of combination therapy.

Specifically, a model for evaluating CITE and/or irAEs of anti-CTLA-4 antibodies, either alone or in combination, using mice with the humanized Ctla4 gene is described herein. In this model, the clinical drug Ipilimumab induced severe irAE, especially when combined with anti-PD-1 antibody. At the same time, another anti-CTLA-4 mAb, L3D10, induced comparable CITE with very mild irAE under the same conditions, showing that irAE and CITE are not intrinsically linked and they demand distinct genetic and immunological bases. The irAE corresponded to systemic T cell activation and reduced Treg/Teff ratios among autoreactive T cells. Using mice that were either homozygous or heterozygous for the human allele, the inventors discovered that irAE required biallelic engagement, while CITE only required monoallelic engagement. As the immunological distinction for monoallelic vs biallelic engagement, the inventors found that biallelic engagement of Ctla4 gene was necessary for preventing conversion of autoreactive T cells into Treg. Humanization of L3D10 that led to loss of blocking activity further increased safety without affecting the therapeutic effect. Taken together, the data presented herein demonstrate that complete CTLA-4 occupation, systemic T cell activation and preferential expansion of self-reactive T cells are dispensable for tumor rejection but correlate with irAE, while blocking B7-CTLA-4 interaction impacts neither safety nor efficacy of anti-CTLA-4 antibodies. These data provide important insights for clinical development of safer and potentially more effective CTLA-4 targeting immunotherapy.

Described herein are important principles relevant to anti-CTLA-4 mAbs-induced irAE. In particular, anti-CTLA-4 mAbs with strong binding affinity of CTLA-4 at low pH, like Ipilimumab or Tremelimumab, will drive surface CTLA-4 to lysosomal degradation during internalization, which trigger irAEs as a result of the loss of surface CTLA-4. In contrast, anti-CTLA-4 mAbs with weak binding affinity in low pH, will dissociate from CTLA-4 during antibody-induced internalization. Internalized CTLA-4 will be released from these antibodies and recycle back to cell surface and maintain the function of CTLA-4 as a negative regulator of immune response. By preserving cell surface CTLA-4, which is the target for ADCC/ADCP for intratumor Treg depletion, pH sensitive antibodies are more effective in selective Treg depletion in tumor microenvironment and thus in rejecting large tumors. These findings represent a significant paradigm shift in CTLA-4 targeting for the development of therapeutic agents, from one that selects antibodies based on antagonizing the interaction between B7 and CTLA-4 to one that preserves normal CTLA-4 recycling. This provides important innovations to the design and/or selection of novel anti-CTLA-4 antibodies with better anti-tumor efficacy and lower toxicity.

Specifically, to increase the anti-tumor activity, CTLA-4 targeting agents will deplete Tregs in the tumor microenvironment. In a particular embodiment, the anti-CTLA-4 mAbs have increased Fc mediated Treg depleting activity. Treg depletion can occur by antibody-dependent cell-mediated cytotoxicity (ADCC) or antibody-dependent cell-mediated phagocytosis (ADCP). This activity can also be enhanced if the CTLA-4 antibody does not down regulate CTLA-4 of regulatory T cells in the tumor microenvironment, preferentially by preserving recycle of internalized CTLA-4 molecules.

To reduce irAEs, CTLA-4 targeting agents will be selected or engineered to preserve normal CTLA-4 recycle and thus its normal function of regulatory T cells outside the tumor microenvironment. In a particular embodiment, the anti-CTLA-4 mAbs have substantially reduced binding affinity to CTLA-4 at late endosomal or lysosomal pH (pH4-6) and will dissociate from CTLA-4 during antibody-induced internalization, allowing released CTLA-4 to recycle back to the cell surface and maintain the function of CTLA-4 as a negative regulator of immune response.

In most preferred embodiments, anti-CTLA-4 antibodies are selected or engineered to improve both Treg depleting anti-tumor activity and CTLA-4 recycling activity.

To further enhance the toxicity profile of the CTLA-4 targeting agents, they may have reduced binding to soluble CTLA-4 (sCTLA-4). sCTLA-4 is generated by alternative splicing of the CTLA-4 gene transcript, and there is an association between CTLA4 polymorphism and multiple autoimmune diseases relates to the defective production of soluble CTLA4 (nature 2003, 423: 506-511) and genetic silencing of the sCTLA4 isoform increased the onset of type I diabetes in mice (Diabetes 2011, 60:1955-1963). For example, genetic variants that generate less sCTLA-4 transcript, such as haplotype CT60G, have increased autoimmune disease-susceptibility relative to haplotypes that generate more sCTLA-4, such as the resistant CT60A haplotype. Accordingly, the presence of sCTLA-4 in the serum is associated with reduced autoimmune disease. Furthermore, soluble CTLA4 (abatacept and belatacept) is a widely used drug for immune suppression. Therefore, anti-CTLA-4 mAbs with reduced binding affinity to sCTLA-4 may maintain the function of sCTLA-4 as a negative regulator of immune response. The invention described herein also includes designing novel anti-CTLA-4 antibodies or enhancing the efficacy and/or toxicity profile of existing anti-CTLA-4 antibodies by incorporating the functional characteristics or attributes of the antibodies described herein.

Provided herein is an anti-CTLA-4 antibody, which may not confer systemic T cell activation or preferential expression of self-reactive T cells, and/or which may allow CTLA-4 to cycle back to a cell surface. The antibody may bind to CTLA-4 with a higher affinity at pH 7.0 as compared to a pH of 5.5 or 4.5. The antibody may induce Fc-R-mediated T regulatory cell depletion in a tumor microenvironment. The antibody may not confer systemic T cell activation or preferential expression of self-reactive T cells. The foregoing antibody may not block binding of CTLA-4 to its B7 ligand. The antibody may have reduced affinity to soluble CTLA-4 compared to CTLA-4 located on the cell surface. The anti-CTLA-4 antibody may be combined with an anti-PD-1 or anti-PD-L1 antibody. The anti-CTLA-4 antibody may be used for treating cancer.

Also provided herein is a method of identifying an anti-CTLA-4 antibody that induces lower levels of immunotherapy-related adverse events. The method may comprise providing cells comprising cell surface CTLA-4, contacting the cells with a candidate anti-CTLA-4 antibody, following a period of incubation, detecting the amount of cell surface CTLA-4, and comparing the amount of cell surface CTLA-4 to a threshold level. The threshold level may be the amount of cell surface CTLA-4 from cells that were contacted with a control anti-CTLA-4 antibody. A higher amount of cell surface CTLA-4 as compared to the threshold level may identify the candidate anti-CTLA-4 antibody as an anti-CTLA-4 antibody that induces lower levels of irAE. The cells may express human CTLA-4, and the cell surface CTLA-4 may be detectably labeled. The detectable label may be a fluorescent tag, such as orange fluorescent protein. The detecting may comprise measuring the amount of the detectable label of the cell surface CTLA-4 using a Western blot, immunohistochemistry, or flow cytometry, The incubation may comprise contacting the candidate anti-CTLA-4 antibody with a detectably labeled anti-IgG antibody, and measuring the amount of the detectable label of the detectably labeled anti-IgG antibody using a Western blot, immunohistochemistry or flow cytometry. The detectably labeled anti-IgG antibody may comprise alex488. The cells may be 293T cells, Chinese Hamster Ovary cells, and T regulatory cells (Tregs).

Further provided herein is an anti-CTLA-4 antibody that has higher binding affinity for CTLA-4 at a high pH of 6.5-7.5 as compared to a low pH of less than or equal to 6. The high pH may be 7 and the low pH may be 4.5 or 5.5.

Also provided herein is a method of screening for or designing an anti-CTLA-4 antibody for use in immunotherapy, where the anti-CTLA-4 antibody does not cause lysosomal CTLA-4 degradation. The method may comprise (a) contacting the anti-CTLA-4 antibody with a CTLA-4 protein at a pH of 6.5-7.5, and quantifying the amount of anti-CTLA-4 antibody binding to the CTLA 4 protein; (b) contacting the anti-CTLA-4 antibody with a CTLA-4 protein at a pH of 4.5-5.5, and quantifying the amount anti-CTLA-4 antibody binding to the CTLA-4 protein; (c) comparing the amount of binding in (a) and (b). The anti-CTLA-4 antibody may not cause lysosomal CTLA-4 degradation if the amount of binding in (a) as compared to (b) is greater than or equal to a threshold level. The pH of (a) may be 7.0, the pH of (b) may be 5.5, and the threshold level may be 3-fold. The pH of (a) may be 7.0, the pH of (b) may be 4.5, and the threshold level may be 10-fold. The amount of anti-CTLA-4 antibody binding may be the amount of anti-CTLA-4 antibody required to achieve 50% maximal binding to the CTLA-4 protein. The anti-CTLA-4 antibody may allow CTLA-4 that has been bound at a cell surface to recycle back to the cell surface after endocytosis.

Further provided herein is a method of treating cancer in a subject in need thereof, which may comprise administering to the subject an antibody whose binding to CTLA-4 is disrupted at an acidic pH corresponding to that found in endosomes and lysosomes. The anti-CTLA-4 antibody may exhibit a reduction of at least 3-fold in its binding to CTLA-4 at pH 5.5 as compared to pH 7.0, and may exhibit a reduction of at least 10-fold in its binding to CTLA-4 at pH 4.5 as compared to pH 7.0. The anti-CTLA-4 antibody may exhibit a greater reduction in binding to soluble CTLA-4 than to cell-surface-bound or immobilized CTLA-4, as compared to Ipilimumab or Tremelimumab.

Also provided herein is an anti-CTLA-4 antibody identified, screened or designed as described herein. The anti-CTLA-4 antibody may be administered to a subject in need thereof in a method of treating cancer, may be used to treat cancer, and may be used in the manufacture of a medicament for treating cancer. The anti-CTLA-4 antibody may be used in combination with an anti-PD-1 or anti-PD-L1 antibody, and the antibodies may be administered concomitantly or sequentially, and may be combined into a single composition.

### BRIEF DESCRIPTION OF THE DRAWINGS

FIG. 1. Mutational analysis of CTLA-4-Fc reveals that Ipilimumab and L3D10 bind to distinct but overlapping epitopes. a-e. Based on the crystal structure and variation of mouse and human CTLA-4 sequences, hCTLA-4-Fc mutants M17 (SEQ ID NO: 1) and M17-4 (SEQ ID NO: 2) were generated. a. The integrity of CTLA-4 molecules was confirmed by their ability to bind to biotinylated B7-1. Control hIgG-Fc, WT (M1) and mutated (M17 and M17-4) hCTLA-4-Fc proteins were coated on 96-well plate at a concentration of 1 μg/ml. Varying doses of biotinylated hB7-1-Fc were added to test their binding abilities, which were measured by streptavidin-HRP. b-e. Control hIgG-Fc, WT (M1) (SEQ ID NO: 3) and mutated (M17 and M17-4) hCTLA-4-Fc proteins were coated on 96-well plates at a concentration of 1 μg/mL. Varying doses of biotinylated L3D10 or Ipilimumab were added to test their binding abilities to hCTLA-4-Fc molecules. The specificity of the binding is confirmed by their binding to WT CTLA-4-Fc (b) but not hIgG-Fc (c). While 4 mutations in M17 completely inactivated the binding to both L3D10 and Ipilimumab (d), 3 mutations in M17-4 drastically abrogated the binding to L3D10 but not Ipilimumab (e).

FIG. 2. Ipilimumab exhibits poor blocking activity for B7-CTLA-4 interactions if B7 is immobilized. a. Both Ipilimumab and L3D10 potently block B7-CTLA-4 interaction if soluble B7-1 is used for the binding assay. Varying doses of anti-human CTLA-4 mAbs were added along with 0.025 μg/ml of biotinylated human B7-1-Fc to plate coated with 1 μg/ml human CTLA-4-Fc. The amounts of B7-1-Fc bound to plates were measured using HRP-conjugated avidin. Data shown are means of duplicates and are representative of two independent experiments. b. Ipilimumab binds better than L3D10 to biotinylated human CTLA-4-Fc. Varying doses of anti-human CTLA-4 mAbs or control IgG were coated onto the plate. Biotinylated CTLA4-Fc was added at 0.25 μg/ml. The amounts of CTLA-4 bound to plates were measured using HRP-conjugated streptavidin. Data shown are means of duplicates and are representative of two independent experiments. c. Detectable but modest blocking of mouse B7-1-human CTLA-4 interaction by Ipilimumab when mB7-1 is expressed on CHO cells. Varying doses of anti-human CTLA-4 mAbs were added along with 200 ng of human CTLA-4-Fc to 1.2×10⁵ CHO cells expressing mouse B7-1. In contrast, L3D10 showed strong blocking of binding of mouse B7-1 to human CTLA-4. Data shown are means and S.D. of triplicate data and are representative of three independent experiments. d. L3D10 but not Ipilimumab blocks interaction between polyhistindine tagged human CTLA-4 and CHO cells expressing human B7-1. 1.2×10⁵ CHO cells expressing human B7-1 were incubated with 200 ng biotinylated and polyhistidine-tagged CTLA-4 along with given doses of antibodies. The amounts of CTLA-4-Fc bound to CHO cells were detected with PE-streptavidin by flow cytometry. Data (Mean±S.D.) shown are normalized mean fluorescence intensity (MFI) of triplicate samples and are representative of two independent experiments. e. Ipilimumab and L3D10 exhibited differential blocking activity for the interaction between soluble hCTLA-4 and cell surface expressed hB7-1. hB7-1-positive, FcR-negative L929 cells (1×10⁵/test) were incubated with biotinylated CTLA-4-Fc (200 ng/test) along with given doses of antibodies. The amounts of B7-bound CTLA-4-Fc were detected with PE-streptavidin, and mean fluorescence intensity (MFI) of PE was calculated. Data represent the results of two independent experiments.

FIG. 3. Ipilimumab exhibits poor blocking activity for B7-1-CTLA-4 and B7-2-CTLA-4 interactions if the B7-1 or B7-2 are immobilized. (A-C) Blocking activities of anti-human CTLA-4 mAbs Ipilimumab and L3D10 in B7-1-CTLA-4 interaction. (A) hB7-1-Fc was immobilized at the concentration of 0.5 μg/ml. Biotinylated CTLA-4-Fc was added at 0.25 μg/ml along with given doses of antibodies. (B) As in A, except that varying doses of biotinylated CTLA-4-Fc was used in the presence of a saturating dose of Ipilimumab or L3D10 (100 μg/ml). (C) As in A, except that varying doses of B7-1-Fc were used to coat plate and a saturating dose of Ipilimumab or L3D10 (100 μg/ml) was used to block CTLA-4-B7-1 interaction. (D-F) Blocking activities of anti-human CTLA-4 mAbs Ipilimumab and L3D10 in B7-2-CTLA-4 interaction. (D) As in A, except that hB7-2-Fc was immobilized. (E), As in B, except that hB7-2-Fc was immobilized. (F) As in C, except that hB7-2-Fc was immobilized. Data shown in A-F are means of duplicate or triplicate optical density at 450 nm. (G) Blocking of CTLA-4 interaction with cell surface hB7-1. CHO cells expressing hB7-1 were incubated with biotinylated CTLA-4-Fc along with given doses of antibodies. The amounts of B7-bound CTLA-4-Fc were detected with PE-streptavidin, and mean fluorescence intensity (MFI) of PE was calculated. (H) Blocking of CTLA-4 interaction with cell surface mB7-2. As in C, except CHO cell expressing mB7-2 was used. (I) Blocking of CTLA-4-Fc binding to spleen DCs matured with overnight LPS stimulation. As in G and H, except 0.5 μg/ml LPS-stimulated 2×10⁶ splenocytes were used for each test and CD11c^{high} DCs (as FIG. 5B) were gated for analyzing PE intensity. Data (Mean±S.D.) shown are normalized MFI values of triplicate samples. Data shown in this figure have been repeated 2-5 times.

FIG. 4. Reconciling the differential blocking effects of Ipilimumab. (A-D) Ipilimumab does not break up preformed B7-CTLA-4 complex. (A, B) Impact of anti-CTLA-4 mAbs on B7-complexed CTLA-4. The B7-CTLA-4 complexes were formed by adding biotinylated CTLA-4 to plates pre-coated with either B7-1 (A) or B7-2 (B). Grading doses of anti-CTLA-4 mAbs were added to plates with pre-existing B7-1-CTLA-4 complex (A) or B7-2-CTLA-4 complex (B). Two hours later, the unbound proteins were washed away and the amounts of B7-1 or B7-2-complexed CTLA-4 were detected using HRP-labeled Streptavidin. (C) Dissociation kinetics of B7 and CTLA-4 complex based on flow cytometric assays using B7-expressing CHO cells. Surface hB7-1 or mB7-2 expressing CHO cells (1×10⁵/test) were incubated with soluble biotinylated CTLA-4-Fc (200 ng/test) for 30 min at room temperature. After washing, cells were incubated in 100 μl DPBS buffer for the indicated minutes. The amounts of B7-bound CTLA-4-Fc were detected with PE-streptavidin by flow cytometry, and the mean fluorescence intensity (MFI) of PE was calculated from triplicated samples. Data shown are the results from one of two independent experiments. (D) L3D10 but not Ipilimumab significantly disrupts the pre-established interaction between soluble CTLA-4 and hB7-1 expressed on CHO cells. Surface hB7-1 expressing CHO cells (1×10⁵/test) were incubated with soluble biotinylated CTLA-4-Fc (200 ng/test) for 30 min at room temperature. After washing, cells were incubated with given doses of antibodies in 100 μl DPBS buffer for 1 hour. The amounts of B7-bound CTLA-4-Fc were detected with PE-streptavidin, and MFI of PE was calculated. The results represent one of three independent assays with similar patterns. (E) Ipilimumab does not relieve CTLA-4-Fc mediated inhibition of CD28-Fc binding to B7-1-transfected J558 cells (J558-B7). J558-B7 cells were incubated with biotinylated CD28-Fc (20 μg/ml) in the presence of CTLA-4-Fc (5 μg/ml) and grading doses of anti-CTLA-4 mAbs or control IgG-Fc. Data shown are means and S.E.M. of MFI from triplicate samples and are representative of at least three independent experiments with similar results. (F) Kinetics of B7-1-CTLA-4 interaction when B7-1 was immobilized. (G) Kinetics of B7-1-CTLA-4 interaction when CTLA-4 is immobilized. Data shown in this figure were repeated 2-5 times.

FIG. 5. Characterization of cellular assays for B7-CTLA-4 interactions. a. Confocal images of 293T cells stably expressing wild-type (WT, top panels) and Y201V mutant (bottom panels) of human hCTLA-4-OFP proteins. Note that while WT hCTLA-4 is predominantly intracellular, mutant hCTLA-4 molecules show a clear pattern of plasma membrane distribution. b. GFP⁺OFP⁺ cells in cell-cell binding assays used in FIG. 6 are cell-cell aggregates based on their forward and side scatters. Representative flow profiles of hB7-2-GFP-CHO and hCTLA-4^{Y201V}-OFP-293T cells co-incubated at 4° C. for 2 h. Top panels show forward vs. side scatters of the GFP⁺OFP⁺ cells, while the lower panels show comparisons of the forward scatters (left) and side scatters (right) of single vs. double positive cells. c. Characterization of the transendocytosis assay. The top panels show the gating used for data presented in FIG. 7, while the lower panels show that after co-incubation at 37° C. for 4 hours, CTLA-4-OFP-CHO cells acquired GFP signals from hB7-2-GFP-CHO cells without alteration in the forward and side scatters.

FIG. 6. Ipilimumab is ineffective in blocking B7/CTLA-4 mediated cell-cell interactions. (A) Profiles of B7-1-GFP or B7-2-GFP-transfected CHO cells or CTLA-4^{Y201V}-transfected 293T cells or mixture of B7-2 and CTLA-4 transfectants without co-incubation. (B) SDS-PAGE analysis for purity of Fabs used for the study. (C, D) Representative FACS profiles (C, Fabs used at 10 μg/ml) and dose responses (D) showing comparable binding by L3D10 and Ipilimumab Fabs to CTLA-4-OFP transfected CHO cells. Alex Fluor 488-conjugated goat anti-human IgG (H+L) was used as the secondary antibody for the binding assay. Dose responses show similar binding activity of Ipilimumab and L3D10 Fabs. AF488-MFI, mean fluorescence intensity of Alex Fluor 488 dye. (E) Inhibition of B7-1-CTLA-4^{Y201V}-mediated cell-cell interaction by anti-CTLA-4 mAb Fabs. B7-1-GFP-transfected CHO cells and CTLA-4^{Y201V}-transfected 293T cells were co-incubated at 4° C. for 2 hours in the presence of 10 μg/ml Fab or control proteins. Data shown are representative FACS profiles. (F) Quantitative comparison between L3D10 and Ipilimumab for their blocking of cell-cell interaction mediated by B7-1 and CTLA-4 expressed on opposing cells. As in E, except that grading doses of antibodies were added. (G) Inhibition of B7-2-CTLA-4^{Y201V}-mediated cell-cell interaction by anti-CTLA-4 mAb Fabs. As in E, except that B7-2-GFP transfectants were used. (H) Quantitative comparison between L3D10 and Ipilimumab for their blocking of cell-cell interaction mediated by B7-2 and CTLA-4 expressed on opposing cells. As in F, except that B7-2-GFP-transfected CHO cells were used. All assays were repeated at least 2 times.

FIG. 7. Ipilimumab is ineffective in blocking B7-transendocytosis by CTLA-4. (A) FACS profiles of B7-2-GFP- or CTLA-4-OFP-transfected CHO cell lines used for transendocytodosis assay. (B) Rapid transendocytosis of B7-2 by CTLA-4. B7-2-GFP transfectants and CTLA-4-OFP-transfectants were co-incubated for 0, 0.5, 1 and 4 hours at 37° C. (C) Lack of transendocytosis of B7-H2 by CTLA-4. As in B, except that B7-H2-GFP transfected P815 cells and data at 0, 1 and 4 hours of co-culturing are presented. (D) Representative profiles depicting differential blockade of transendocytosis of B7-1-GFP by CTLA-4-OFP-expressing CHO cells during coculture in the presence of control hIgG-Fc or Fab from either Ipilimumab or L3D10 (10 μg/ml) for 4 hours. (E) Dose response curve depicting inhibition of B7-1 transendocytosis by L3D10 and Ipilimumab Fab. As in D, except varying doses of control hIgG-Fc or Fab were added to the co-culturing. (F) As in D, except that B7-2-GFP-transfected CHO cells were used. (G) Dose response curve depicting inhibition of B7-2 transendocytosis by L3D10 and Ipilimumab Fab. As in E, except that B7-2-GFP transfected CHO cells were used. Data shown (Mean±S.D.) are % of transendocytosis over varying doses of Fab. All assays were repeated at least 3 times.

FIG. 8. Ipilimumab does not block B7-CTLA-4 interaction in vivo. (A) Diagram of the experimental design. (B) Representative data showing the phenotype of CD11b⁺CD11c^{high} dendritic cells (DC) analyzed for B7 expression. (C) Representative histograms depicting the levels of mB7-1 on DC from mice that received control hIgG-Fc, L3D10 or Ipilimumab. Data in the top panel show an antibody effect in homozygous human CTLA4 knockin mice (Ctla4^{h/h}), while that in the bottom panel show an antibody effect in the heterozygous mice (Ctla4^{h/m}). (D) As in C, except that expression of mB7-2 is shown. Data shown in c and d are representative of those from 3 mice per group and were repeated once. (E) In human CTLA4 homozygous mice, L3D10 but not Ipilimumab induced upregulation of mB7-1 (left panel) and mB7-2 (right panel). Data shown (mean±S.E.M.) are summarized from two experiments involving a total of 6 mice per group. (F) As in E, except that heterozygous mice are used. Neither L3D10 nor Ipilimumab block B7-CTLA-4 interaction in mice that co-dominantly express both mouse and human Ctla4 genes. Statistical significance was determined using Student's t test. *P<0.05, **P<0.01, ***P<0.001. n.s., not significant.

FIG. 9. Despite somewhat higher levels of endotoxin detected in the hIgG-Fc control preparation than the anti-CTLA4 antibody preparations, hIgG-Fc did not up-regulate B7-1 and B7-2 expressions on mouse spleen DCs. a, b. Representative profiles of B7-1 (a) and B7-2 (b) expression among the spleen DCs gated as depicted in FIG. 5b from Ctla4^{h/h} mice treated with 500 μg of hIgG-Fc or equal volume of PBS. c, d. Summarization of mean fluorescence intensities for B7-1 (c) and B7-2 (d) expressed on spleen DCs. n=5 Ctla4^{h/h} mice for each group. Therefore, the profiles of the control hIgG-Fc-treated mice reflect the basal expression levels of B7-1 and B7-2. Thus, the lack of effect of Ipilimumab over hIgG-Fc indicates its inability to up-regulate B7-1 and B7-2 in vivo as shown in FIG. 8.

FIG. 10. L3D10, HL12, HL32 and Ipilimumab bind to human CTLA-4 but not mouse Ctla-4. Data shown are dot plots of intracellular staining of CTLA-4 among gated CD3⁺CD4⁺ cells, using spleen cells from Ctla4^{h/h} (top) or Ctla4ⁿ (bottom) mice. Anti-mouse Ctla-4 mAb 4F10 (BD Biosciences) was used as control.

FIG. 11. Ipilimumab does not block human B7-human CTLA-4 interaction in vivo. (A) FACS profiles depicting the composition of human leukocytes among the peripheral blood leukocytes (PBL) of NSG™ mice reconstituted with human cord blood CD34⁺ cells. (B) Summary data of individual mice as analyzed in A. (C) Normal composition of Tregs (middle right panel) and DCs (right panel) in spleen of humanized NSG™ mice. (D) Expression of FOXP3 and CTLA-4 among human CD4 T cells in mice spleen. (E, F) L3D10 but not Ipilimumab blocks human B7-2-human CTLA-4 interaction in the human cord blood CD34⁺ stem cell reconstituted NSG™ mice. The humanized mice received intraperioneal treatment of either control Ig or anti-CTLA-4 mAbs (500 μg/mouse). Splenocytes were harvested at 24 hours after injection and analyzed for expression of B7-2 on DC. (E) Representative profiles of hB7-2 on DC. (F) Summary data (mean±S.E.M.) from two independent experiments. The mean data in the control mice is artificially defined as 100 and those in experimental groups are normalized against the control. Statistical significance was determined using Student's t test. *P<0.05, **P<0.01, ***P<0.001. n.s., not significant.

FIG. 12. Blocking the B7-CTLA-4 interaction does not contribute to anti-CTLA-4 mAbs elicited cancer immunotherapeutic activity and intratumorial Treg depletion. (A) Comparable immunotherapeutic effect despite vastly different blocking activity by two anti-CTLA-4 mAbs. 5×10⁵ or 1×10⁶ MC38 tumor cells were injected (s.c.) into Ctla4^{h/h} mice (n=5-6), and mice were treated (i.p.) with 100 μg (left), 30 μg (middle) or 10 μg (right) Ipilimumab, L3D10 or control hIgG-Fc per mouse on days 7, 10, 13, and 16, as indicated by arrows. Data represent mean±S.E.M. of 5-6 mice per group. Statistical analyses were performed by two-way repeated measures ANOVA (treatment×time). For 100 μg treatments, Ipilimumab vs. hIgG-Fc: P<0.0001; L3D10 vs. hIgG-Fc: P<0.0001; Ipilimumab vs. L3D10: P=0.0699. For 30 μg treatments, Ipilimumab vs. hIgG-Fc: P<0.0001; L3D10 vs. hIgGFc: P<0.0001; Ipilimumab vs. L3D10: P=0.9969. For 10 μg treatments, Ipilimumab vs. hIgG-Fc: P<0.0001; L3D10 vs. hIgG-Fc: P<0.0001; Ipilimumab vs. L3D10: P=0.9988. Data are representative of 3-5 independent experiments. (B) Ipilimumab and L3D10 have similar therapeutic effect for B16 melanoma growth. 1×10⁵ B16 tumor cells were injected (s.c.) into Ctla4^{h/h} mice (n=4-5), and mice were treated (i.p.) with 100 μg (left) or 250 μg (right) Ipilimumab, L3D10 or control hIgG-Fc on day 11, 14, 17 (left) or on day 2, 5, and 8 (right), as indicated by arrows. For the left panel, Ipilimumab vs. hIgG-Fc: P=0.0265; L3D10 vs. hIgG-Fc: P=0.0487; Ipilimumab vs. L3D10: P=0.302. For the right panel, Ipilimumab vs. hIgG-Fc: P=0.00616; L3D10 vs. hIgG-Fc: P=0.0269: Ipilimumab vs. L3D10: P=0.370, Data represent mean±S.E.M. of 4-5 mice per group. (C-F) Blocking B7-CTLA-4 interactions does not contribute to selective depletion of Treg in tumor microenvironment in the Ctla4^{h/h} mice. L3D10 and Ipilimumab did not delete Treg in the spleen (C) of mice at 3 days after third treatment. Data shown are the percentage of Foxp3⁺ cells among CD4 T cells in Ctla4^{h/h} mice. n=6 mice for each group. Both L3D10 and Ipilimumab depleted Treg in tumors transplanted into the Ctla4^{h/h} mice, as determined by % Treg among CD4 T cells (D, upper), absolute Treg number (D, lower) and CD8/Treg ratios (E). Summary data from two experiments involving 7 mice per group are presented in D (upper panel) and E. The numbers of Foxp3⁺ cells (d, lower panel) in the tumor from Ctla4^{h/h} mice were counted by flow cytometry on 3 days after the third antibody treatment. n=5 for each group. Statistical analyses were performed by ordinary one-way ANOVA with Tukey's multiple comparisons test. (F) Blocking B7-CTLA-4 interaction does not contribute to increased IFNγ producing cells among tumor-infiltrating CD4 (left) or CD8 (right) T cells. Summary data are from two experiments involving 7 mice per group. Single cell suspensions of collagenase-digested tumors from mice were prepared between 13 or 16 days and cultured in the presence of Golgi blocker for 4 hours and stained for intracellular cytokines. (G-J) In Ctla4^{h/m} mice where neither antibody blocks the B7-CTLA-4 interaction, both L3D10 and Ipilimumab induce robust tumor rejection and intratumorial Treg depletion. As in A, except that heterozygous mice that express both mouse and human CTLA-4 were used. (G, H) Both higher doses (G, 100 μg/mouse/injection) and lower doses (H, 10 μg/mouse/injection) of antibody treatments showed effective therapeutically effects. In G, Ipilimumab vs. hIgG-Fc: P<0.0001; L3D10 vs. hIgG-Fc: P<0.0001; Ipilimumab vs. L3D10: P=0.4970. Data are representative of 5 independent experiments. Tregs were selectively depleted in the tumor (I) but not in the spleen (J) of Ctla4^{h/m} mice that neither antibodies significantly blocked B7-CTLA-4 interaction in vivo. Data (Mean±S.E.M.) shown in C, D, E and I are the percentage of Treg at 18 (experiment 1) or 20 days (experiment 2) after tumor cell challenge and 11 or 13 days after initiation of 3 or 4 anti-CTLA-4 mAb treatments as indicated in arrows. Statistical significance in C-F and I-J was determined using the Mann-Whitney test. (K) Anti-FcR mAb administration abrogated the therapeutic effect of Ipilimumab. 5×10⁵ MC38 tumor cells were injected (s.c.) into Ctla4^{h/h} mice, and mice were treated (i.p.) with 30 μg Ipilimumab alone, or 30 μg Ipilimumab (black arrow) plus 1 mg 2.4G2 (red arrow) or control hIgG-Fc on days 7, 10, 13, and 16, as indicated. Statistical analyses were performed by two-way repeated measures ANOVA (treatment×time). Ipilimumab vs. hIgG-Fc: P=0.0003; Ipilimumab plus 2.4G2 vs. hIgG-Fc: P=0.6962; Ipilimumab plus 2.4G2 vs. Ipilimumab: P=0.0259.

FIG. 13. CTLA-4 is expressed in tumor-infiltrated Tregs. a. Tumor-derived FoxP3⁺ Tregs had higher expression of CTLA-4 than Foxp3-negative CD4 T cells. As in FIG. 12, MC38 tumor cells were injected into Ctla4^{h/h} or Ctla4^{h/m} mice and mice were treated with 100 μg per dose of control hIgG-Fc or anti-CTLA-4 mAbs on days 7, 10, and 13. Five days after the third antibody treatment, mice were sacrificed and tumor cells were subjected to flow cytometric analysis for human CTLA-4 or mouse Ctla-4 expression in tumor-infiltrated CD45⁺CD4⁺Foxp3⁺ Tregs and CD45⁺CD4⁺Foxp3⁻ T cells. Data represent the results from one of three independent experiments. b, c. Tregs from tumor had higher expression of both surface CTLA-4 and total CTLA-4 than that from spleen. As in FIG. 12, 14 days after MC38 tumor inoculation, Ctla4^{h/h} mice were sacrificed for flow cytometric analysis of surface CTLA-4 (b) and total CTLA-4 (c) expression in spleen and tumor derived CD4⁺Foxp3⁺ Tregs. Each line of the histogram plots indicates one individual mouse. n=6 mice and data shown represent the results from one of at least three independent experiments.

FIG. 14. Effects of anti-hCTLA-4 mAbs on IFNγ and TNFα production among spleen and tumor T cells. As in FIG. 12a, MC38 tumor cells were injected into Ctla4^{h/h} mice and mice were treated with 100 μg per dose of control hIgG-Fc or anti-CTLA-4 mAbs on days 7, 10, and 13. Three days after the third antibody treatment, mice were sacrificed to analyze the frequencies of IFNγ- and TNFα-expressing cells among CD4 (a, c, e) and CD8 (b, d, f) T cells in tumors (a, b) and spleens (c-f) from the treated mice. Summary data are from two experiments involving 7 mice per group.

FIG. 15. Humanized L3D10 antibody progenies (HL12 and HL32) that lost blocking activities remain effective in local Treg depletion and tumor rejection. (A) Binding activities of HL12, HL32 and L3D10 to 1 μg/ml immobilized polyhistidine-tagged CTLA-4. (B) HL12 and HL32 failed to block B7-1-CTLA-4 interaction. B7-1-Fc was immobilized at a concentration of 0.5 μg/ml. Biotinylated CTLA-4-Fc was added at 0.25 μg/ml along with grading concentration of anti-CTLA-4 mAbs. (C) HL12 and HL32 barely block B7-2-CTLA-4 interaction. As in B, except B7-2-Fc is immobilized. (D) HL12 and HL32 failed to up-regulate B7-1 and B7-2 in vivo. As in FIG. 8, Ctla4^{h/h} mice received 500 μg/mouse/injection of control hIgG-Fc or anti-CTLA-4 mAbs. Spleen cells were harvested the next day to determine the levels of B7-1 and B7-2 on CD11b⁺CD11c^{high} DCs, as detailed in FIG. 8. n=3 for each group. (E-G) Similar to L3D10, HL12 and HL32 showed selective depletion of Tregs in the tumor microenvironment in the Ctla4^{h/h} mice. As in FIG. 12, L3D10, HL12 and HL32 elicited comparable and efficient depletion of Tregs in tumor (E), but did not deplete Tregs in spleen (F) and tumor draining lymph node (G). Data shown were pooled from 2 experiments. n=5 mice for each group. Mice were sacrificed one day after one injection of 100 μg indicated drug. (H) Efficient rejection of MC38 tumors by Ipilimumab and humanized L3D10 antibodies HL12 and HL32. Mice bearing MC38 were treated on days 7, 10, 13 and 16 days after tumor cells inoculation with 100 μg control IgG-Fc or Ipilimumab or HL12, HL32. Data shown are means and S.E.M. of tumor volume. n=6 mice for each group. Statistical analyses were performed by two-way repeated measures ANOVA (treatment×time). Ipilimumab vs. hIgG-Fc: P=0.034; HL12 vs. hIgG-Fc: P=0.037; HL32 vs. hIgG-Fc: P=0.0336; HL12 vs. Ipilimumab: P=0.9021; HL32 vs. Ipilimumab: P=0.9972; HL32 vs. HL12: P=0.7250. (I) HL32 and L3D10 are comparably effective in the treatment of B16 tumor cells in a minimal disease model. 1×10⁵ B16 tumor cells were injected (s.c.) into Ctla4 mice (n=4-5), and mice were treated (i.p.) with 250 μg of Ipilimumab, L3D10, HL32 or control IgG-Fc on days 2, 5, and 8. HL32 vs. hIgG-Fc: P=0.0002; L3D10 vs. HL32:P=0.9998; Ipilimumab vs. HL32: P=0.8899. Data represent mean±S.E.M. of 5-6 mice per group.

FIG. 16. Despite the inability to block CTLA-4-B7 interaction, HL12 and HL32 exhibit similar effects as L3D10 on abundance of T cell subpopulations in peripheral lymph organs and tumors. a. The ability of HL12 and HL32 to block soluble B7 binding to immobilized CTLA-4-Fc was abrogated. hCTLA-4-Ig was immobilized at the concentration of 0.25 μg/ml on 96-well ELISA plate. Biotinylated hB7-1-Fc was added at 0.25 μg/ml along with giving doses of anti-CTLA-4 mAbs (L3D10, HL12 and HL32) or control hIgG-Fc. After washing, the plate-bound biotinylated hB7-1-Fc was detected with HRP-conjugated avidin. Data shown are means of triplicate optical density at 450 nm. Results are representative of 3 independent experiments. b, c. As L3D10, HL12 and HL32 preferentially eliminate tumor-infiltrated Tregs. As in FIGS. 12e-12g, the frequencies (b) and numbers (c) of CD8 T cells (top row), CD4⁺Foxp3⁻ T cells (middle row) and CD4⁺Foxp3⁺ Tregs (bottom row) in tumor, spleen and tumor draining lymph node (dLN) were analyzed. Live CD45⁺ leukocytes were initially gated to quantitate the frequencies of T cell subpopulations (T subset/CD45⁺ cells×100%) in various tissues, and the numbers of T cell subpopulations in tumors were normalized against tumor weight (gram). Mice were sacrificed one day after one injection of 100 μg indicated drug. Data shown were pooled from 2 experiments. n=5 mice for each group.

FIG. 17. The therapeutic effect of Ipilumumab is not achieved by blocking CTLA-4-B7 negative signaling. (A) Confirmation of the blocking activities of anti-B7 mAbs. CHO cells expressing mouse B7-1 or B7-2 were incubated with a mixture of antibodies (20 μg/ml) and biotinylated human CTLA-4-Fc (2 μg/ml) for 1 hour. After washing away unbound proteins, the cell surface CTLA-4-Fc was detected by PE-conjugated streptavidin and measured by flow cytometry. Data shown are representative FACS profiles and were repeated 2 times. (B) Diagram of experimental design. MC38 tumor-bearing Ctla4^{h/m} mice received anti-B7-1 and anti-B7-2 antibodies (300 μg/mouse/injection, once every 3 days for a total of 3 injections) in conjunction with either control Ig or Ipilimumab, mice that received Ipilimumab without anti-B7-1 and anti-B7-2 were used as positive control for tumor rejection. (C, D) Saturation of B7-1 and B7-2 by antibody treatments as diagramed in B. The PBL were stained with FITC-conjugated anti-B7-1 and anti-B7-2 mAbs at 24 hours after the last anti-B7 treatment on day 13. PBL from Cd80^{-/-} Cd86^{-/-} mice were used as negative control. (E) Complete blocking of B7-2 in vivo. As in C and D, except that CD45⁺ leukocytes were gated from single cell suspensions of draining lymph nodes in mice bearing MC38 were used. The top panel depicts profiles of B7-2 staining, while the lower panel shows the mean fluorescence intensities. This study has been repeated 3 times. (F) Ablation of antibody responses confirmed the functional blockade of B7 by anti-B7-1 and anti-B7-2 mAbs. Sera were collected at day 22 after tumor challenge to evaluate anti-human IgG antibody response. (G) Saturating blocking by anti-B7-1 and anti-B7-2 mAbs did not affect the immunotherapeutic effect of Ipilimumab. Data shown in g are tumor volumes over time and were repeated twice with similar results. Data in D-G represent mean±S.E.M. n.s., not significant.

FIG. 18. In vivo treatment of anti-B7 mAbs prevents Ipilimumab mediated T cell activation and de novo priming of CD8 T cell. (A) Functional blockade of B7 by anti-B7-1 (1G10) and anti-B7-2 (GL1) mAbs prevented Ipilimumab induced CD4 T cell activation. MC38 tumor-bearing Ctla4^{h/h} mice (n=5 for each group) were treated intraperitoneally with hIgG-Fc (100 μg/mouse/injection), Ipilimumab (100 μg/mouse/injection) or Ipilimumab plus anti-mB7 mAbs (300 μg 1G10 plus 300 μg GL1 per mouse/injection) on days 7, 10 and 13 and euthanized on day 14. Sex and age-matched, tumor-free Ctla4^{h/h} mice were used as control naïve mice. Spleen T cells from these mice were purified by MACS negative selection and co-cultured with naïve spleen DCs in the presence of 10 μg/ml hIgG-Fc for 4 days. The levels of Th2 cytokines (including IL-4, IL-6 and IL-10) in the supernatant were quantitated by cytokine beads assays (CBA). (B, C) Anti-B7 mAbs prevented Ipilimumab induced priming of antigen-specific CD8 T cells. As in A, except that all mice (n=4 for each group) were immunized subcutaneously with 50 μg SIY peptide emulsified in 100 μg Complete Freund's Adjuvant (CFA) on day 8. Mice were sacrificed on day 15 and tumor draining lymph nodes were collected to evaluate SIY-specific CD8 T cells (gated on CD3⁺CD4⁻ cells) by tetramer staining. OVA tetramer was used for control staining. Representative FACS profiles (B) and summary data (C) are shown. Data shown are representative of two independent experiments with similar results.

FIG. 19. Evaluation of blocking activities of commonly used anti-mouse Ctla-4 mAbs 9H10 and 9D9. a, b. 9H10 does not block B7-CTLA-4 interaction if B7-1 (a) and B7-2 (b) are coated onto plates. Biotinylated mouse Ctla-4-Fc fusion protein were incubated with B7-coated plates in the presence of given concentration of control IgG or anti-mouse Ctla-4 mAb 9D9 and 9H10. Data shown are means of duplicated wells and are representative of two independent experiments. c, d. 9D9 and 9H10 exhibit differential binding ability to soluble (c) and plate bound Ctla-4-Fc (d). MPC-11 (mouse IgG2b) and Hamster IgG (Ham IgG) are isotype-matched control Ig proteins. Data shown are means of duplicated wells and are representative of at least two independent experiments. e, f. Differential effect of anti-mouse Ctla-4 mAbs 9D9 and 9H10 on upregulating the levels of B7-1 (e) and B7-2 (f) on splenic CD11c^{high} DCs from WT (Ctla4^{m/m}) mice. At 24 hours after treatment with 500 μg antibodies, mice were sacrificed and splenocytes were harvested for flow staining immediately. IgG group indicates mice receiving 500 μg of MPC-11 and 500 μg of Ham IgG. The data (Mean±S.E.M.) are summarized from 6 independent mice per group in two independent experiments involving 3 mice per group each. Statistical significance in e and f was determined using Student's t test. *P<0.05, **P<0.01, ***P<0.001. n.s., not significant.

FIG. 20. Distinct in vitro and in vivo blocking activities of anti-mouse Ctla-4 mAb 4F10. a, b. The effect of 4F10 on interaction of Ctla-4Fc to plate-coated B7-1 (a) or B7-2 (b). Biotinylated mouse Ctla-4-Fc fusion protein was incubated with B7-coated plates in the presence of given concentrations of control IgG or anti-mouse Ctla-4 mAb 4F10. The Ctla-4-Fc binding was detected with HRP-conjugated avidin. Data shown are means of duplicates and are representative of two independent experiments. c, d. Impact of 4F10 on B7-1 and B7-2 expression on CD11c^{high} dendritic cells. Spleen cells from WT (Ctla4^{m/m}) mice administrated i.p. with 500 μg 4F10 or hIgG-Fc were analyzed for B7 levels by flow cytometry. Summary data (Mean±S.E.M.) on B7-1 (c) and B7-2 (d) levels are from 6 mice per group. The B7 levels in the control IgG-treated group are artificially defined as 100.

FIG. 21. L3D10 and Ipilimumab exhibited comparable anti-tumor activities. MC38-tumor-bearing Ctla4^{h/h} mice (n=5) received treatment of control hIg, Ipilimumab or L3D10 (30 μg/injection×4) on days 7, 10, 13 and 16. The tumor growth was measured every 3 days. Data are mean±S.E.M. and were reproduced more than 3 times. Statistical significance was analyzed by two-way repeat measurement ANOVA with Bonferroni multiple comparison test. hIg vs Ipi, P=0.0335; hIg vs L3D10, P=0.0248; Ipi vs L3D10, P=0.6928.

FIG. 22. Tregs from neonates and adult tumor-bearing mice express higher levels of CTLA-4 molecules than naïve adult mice. A. Comparison between neonates (10 days old male mice, grey line) and adult mice (2-3 months old male mice, black line). Data shown are profiles of Foxp3⁺CD4⁺ Treg from spleen of male mice (n=3). B. Comparison between naïve (black line) and tumor-bearing (grey line) adult male mice (3 months old, n=6). Data shown are FACS profiles depicting distribution of total CTLA-4 among Foxp3⁺CD4⁺ cells. The difference is statistically significant and has been reproduced at least five times.

FIG. 23. Human CTLA4 gene knockin mice distinguished irAE of anti-CTLA-4 mAbs Ipilimumab and L3D10 when used alone or in combination with anti-PD-1 mAb: growth retardation and pure red blood cell aplasia. (A) Time-line of antibody treatment and analysis. C57BL/6 Ctla4^{h/h} mice were treated, respectively, with control human IgG-Fc, anti-human CTLA-4 mAb Ipilimumab, human IgG1 Fc chimeric L3D10+ human IgG-Fc, anti-PD-1 (RMP1-14)+ human IgG-Fc, anti-PD-1+ Ipilimumab, or anti-PD-1+L3D10 at a dose of 100 μg/mouse/injection on days 10, 13, 16 and 19. The CBC analysis was performed on day 41 after birth and necropsy was performed on day 42 after birth. To avoid cage variation, mice in the same cages were individually tagged and treated with different antibodies. Tests were performed double blind. (B) Major growth retardation of female mice by Ipilimumab+ anti-PD-1. One female mouse from the Ipilimumab plus anti-PD-1 treated group was excluded from analysis due to death on day 22 with serious grow retardation. Data shown were means and S.E.M. of % weight gain following the first injection. hIg vs Ipilimumab+ anti-PD-1, P<0.0001; L3D10+ anti-PD-1 vs Ipilimumab+ anti-PD-1, P=0.003. (C) Major growth retardation of male mice by Ipilimumab+ anti-PD-1. As in B, except male mice were used. hIg vs Ipilimumab+ anti-PD-1, P=0.0116; L3D10+ anti-PD-1 vs Ipilimumab+ anti-PD-1, P=0.0152. The numbers of mice used were included in the parentheses following group labels. (D-G) Pure red cell aplasia recapitulated in the mouse model as a typical phenotype of irAE. (D) Ipilimumab+ anti-PD-1 combination therapy reduced hemacrit (HCT), hemoglobin (Hb) and mean corpuscular volume (MCV). Data shown are a summary of 2-3 independent experiments with each dot represents one individual mouse (blue for male mice and red for female mice, and n=9-22 mice per group. (E) Defective generation of red cells in bone marrow. Photographs depict the change of coloration in bone (upper panel) and bone marrow flush (lower panel) in mice that received indicated treatments. (F) Analysis of erythrocyte development by flow cytometry. Data shown are representative FACS profiles depicting distribution of Ter119, CD71 and forward scatters (FSC-A) among bone marrow cells. The gating and % of cells at stage I-V are indicated. (G) Summary data of % of erythroid cells at each of the developmental stages. Data shown are means and S.E.M. of data with 3-4 female mice per group, and were repeated at least three times in both male and female mice. Statistical tests used: B and C, two-way repeat measurement ANOVA with Bonferroni multiple comparison test; D and G, one-way ANOVA with Bonferroni multiple comparison test and Non-Parametric One-way ANOVA (Kruskal-Wallis test) with Dunn's multiple comparisons test.

FIG. 24. Normal blood cell parameters following antibody treatment as outlined in FIG. 23. Data shown are a summary of 2-3 independent experiments with each dot denotes an individual mouse (dark grey for male mice and lighter grey for female mice). CBC results were analyzed by Non-Parametric One-way ANOVA (Kruskal-Wallis test) with Dunn's multiple comparisons test. No statistically significant differences were found in pairwise comparisons. NE, Neutrophils; WBC, White Blood Cells; RBC, Red Blood Cells; MO, Monocytes; LY, Lymphocytes; EO, Eosinophils; RDW, Red Cell Distribution Width; PLT, Platelets; MPV, Mean Platelet Volume.

FIG. 25. Ipilimumab caused heart-defects when used in combination with anti-mouse PD-1. (A) Gross anatomy shows heart enlargement despite reduced body size in mice treated with anti-PD-1+ Ipilimumab. Photographs in the left panels are from formalin-fixed heart from mice that received indicated treatments, and the data on the right panel show the sizes after normalizing against body weight. (B) Macroscopic images depicting enlarged heart atriums and ventricles, and corresponding thinning of heart wall. (C) Histology of control hIg, L3D10+ anti-PD-1 or anti-PD-1+ Ipilimumab-treated hearts. The upper 4 panels show H&E staining at the aorta base, while the lower 4 panels show inflammation in myocardium of the left ventricle. (D) Identification of leukocytes and T cells by immunohistochemistry (top panels) and three-color immunofluorescence staining using FITC-labeled CD4 or CD8, Rhodamine-labeled anti-CD3 or anti-Foxp3 antibodies (lower panel). (E) The composite pathology scores of male and female mice (n=5-12) receiving different treatments. The scores of male mice are indicated with blue circles, while that of female mice are indicated with red circles. The samples were collected from 6 independent experiments and were scored double blind. Data are mean±S.E.M. and analyzed by One-way ANOVA with Bonferroni's multiple comparison test.

FIG. 26. Gross anatomy and H&E staining show hypoplastic ovaries and uterus after Ipilimumab+ anti-PD-1 treatment. As in FIG. 23 and FIG. 25, necropsy was performed on day 42 after birth.

FIG. 27. Ipilimumab increased ACTH levels in sera. C57BL/6 Ctla4 mice were treated, respectively, with control human IgG Fc, anti-PD1, anti-human CTLA-4 mAbs Ipilimumab, L3D10, HL12 or HL32 at a dose of 100 μg/mouse/injection on days 10, 13, 16 and 19. Sera were collected on day 42 or 43 after birth. Serum ACTH levels were measured using Enzyme-linked Immunosorbent Assay Kit for Adrenocorticotropic Hormone (Cloud-Clone Corp., Cat. No. SEA836Mu). n=8-18 mice per group. Statistical significance was analyzed by one-way ANOVA with Bonferroni multiple comparison test.

FIG. 28. Ipilimumab caused multiple organ inflammation when either used as single agent or in combination with anti-PD-1. (A) Representative images of H&E stained paraffin sections from different organs. Representative inflammatory foci are marked with arrows. Scale Bar, 200 μm. (B) Toxicity scores of internal organs and glands. The scores of male mice are indicated with dark grey circles, while that of female mice are indicated with lighter grey circles. (C) Composite scores of all organs and glands. Data are mean±S.E.M., n=5-12 mice per group. The samples were collected from 6 independent experiments and were scored double blind. Data were analyzed by One-way ANOVA with Bonferroni's multiple comparison test.

FIG. 29. Comparing systemic T cell activation in mice that received immunotherapy drugs starting at day 10. (A) Minor impact on CD4 (top panel) and CD8 (bottom panel) T cell frequencies by anti-PD-1 and anti-CTLA-4. Data shown are % of CD4 and CD8 T cells in the spleen on day 32 after the start of antibody treatment. (B) Representative FACS profiles depicting the increase of memory and effector CD4 (Top panels) or CD8 (bottom panels) T cells in mice that received monotherapy and combination treatment of anti-PD-1 plus Ipilimumab during the perinatal period. (C, D) Summary data on the phenotype of CD4 (C) and CD8 (D) T cells in mice that received combination treatments with anti-PD-1 plus anti-CTLA-4 mAbs as indicated. Data shown are % of cells with phenotypes of naïve (left), central memory (middle) and effector (right) memory phenotypes. Data shown are summarized from four experiments involving 7-11 female mice and 2-6 male mice per group. Statistical tests used: A, One-way ANOVA with Bonferroni multiple comparison test; C and D, One-way ANOVA with Bonferroni multiple comparison test.

FIG. 30. Ipilimumab increased the frequency of Treg in the spleen from Ipilimumab-treated mice. C57BL/6 Ctla4^{h/h} mice were treated, respectively, with control human IgG Fc, anti-PD-1 or anti-CTLA-4 mAbs Ipilimumab or L3D10 at a dose of 100 μg/mouse/injection in combination with anti-PD-1 on days 10, 13, 16 and 19. Spleens were collected and the percentages of Foxp3⁺ Treg in splenic CD4 T cells were evaluated by flow cytometry on day 42 after birth. Statistical significance was analyzed by One-way ANOVA with Bonferroni multiple comparison test.

FIG. 31. In combination with anti-PD-1, Ipilimumab preferentially expanded autoreactive Teff. (A) Diagram of the breeding scheme. The mice were produced in two steps. The first step was an outcross between two inbreed strains as indicated. The second step was an intercross of F1s to obtain mice of designed genotypes (H-2^{d+} Ctla4^{h/h or h/m}Mmtv⁸⁺⁹⁺) for the studies. (B) Diagram of the experimental timeline. (C) Representative FACS profiles depicting the distribution of Vβ11, Vβ8 and Foxp3 markers among gated CD4 T cells from mice that received antibody treatments. (D) Composite ratios of Treg/Teff among VSAg-reactive (Vβ5⁺, 11⁺, or 12⁺, top panel) and non-reactive (Vβ8⁺) CD4 T cells. (E) Lack of impact on thymocytes. As in D, except the CD3⁺CD4⁺CD8⁻ thymocytes were analyzed. Data shown are means and S.D., n=6-7 mice per group.

FIG. 32. Ipilimumab binds to human CTLA-4 but not mouse CTLA-4. Data shown are dot plots of intracellular staining of CTLA-4 among gated CD3⁺CD4⁺ cells, using spleen cells from Ctla4^{h/h} (top) or Ctla4^{m/m} (bottom) mice. Biotinylated hIg and Ipilimumab were used for intracellular staining. Anti-CD3 (clone 145-2C11), CD4 (clone RM4-5), FoxP3 (clone FJK-16s) mAbs and FoxP3 staining buffer were purchased from eBioscience.

FIG. 33. Humanized L3D10 clones maintained safety profiles when used in combination therapy with anti-PD-1 mAb. (A) Comparing humanized L3D10 clones HL12 and HL32 with Ipilimumab for their combination toxicity when used during perinatal period. Except changes in antibodies used, the experimental regimen was identical to what was depicted in FIG. 23A. (B) Ipilimumab but not humanized L3D10 clones HL12 and HL32 induced anemia when used in combination with anti-PD-1 antibody. (C) Pathology scores of internal organs and glands after the mice were treated with either control of given combination of immunotherapeutic drugs. (D) Composite pathology scores. Dark grey circles represent scores of male mice and the lighter grey scores represent female mice used. All scorings were performed double blind. Data are mean±S.E.M., n=5-12 mice per group. The samples were collected from 5 independent experiments and were scored double blind. Statistical methods used were: A, Repeated measures two-way ANOVA with Bonferroni's multiple comparison test; B, Non-Parametric One-way ANOVA (Kruskal-Wallis test) with Dunn's multiple comparisons test; C and D, One-way ANOVA with Bonferroni's multiple comparison test.

FIG. 34. Phenotypes of CD4 and CD8 T cells activation in the spleen of humanized mice receiving given immunotherapeutics. Mice were treated as shown in FIG. 23A, except humanized L3D10 clones (HL12 and HL32) were used. Data shown are percentages and phenotypes of CD4 (top panels) and CD8 (Bottom panels) spleen T cells on day 32 after the start of antibody treatment. Data are summarized from 3 experiments involving 5-11 mice (lighter grey: female; dark grey: male) per group. Statistical significance was analyzed by One-way ANOVA with Bonferroni multiple comparison test and Non-Parametric One-way ANOVA (Kruskal-Wallis test) with Dunn's multiple comparisons test.

FIG. 35. Comparing the immunotherapeutic effect of HL12 and HL32 with Ipilimumab. (A, B) MC38 bearing-Ctla4^{h/m} mice (n=5) were i.p. treated with 30 μg (A) or 10 μg (B) of either control hIg, Ipilimumab, HL12 or HL32 on day 7, 10, 13 and 16. (C, D) CT26 bearing-Ctla4^{h/m} mice (n=6-10) were i.p. treated with 150 μg (C) or 100 μg (D) of either control Ig, Ipilimumab, HL12 or HL32 on day 7, 10, 13 and 16. (E, F) B16 bearing-Ctla4^{h/h} mice (n=5-6) were i.p. treated with 250 μg control Ig, Ipilimumab, HL12 (E) or HL32 (F) Data are mean±S.E.M. and data were analyzed by repeated measures two-way ANOVA with Bonferroni's multiple comparison test. In all settings, HL12 and HL32 induced statistically significant tumor rejection when compared with Control hIgG, HL12 (A, P=0.0023; B, P=0.0105; C, P<0.0001; D, P=0.0272; E, P<0.0001); HL32 (A, P=0.004; B, P=0.0059; C, P<0.0001; D, P=0.0259; F, P=0.1003). Tumor rejections induced by Ipilimumab were also significant in all but except one (C) setting (A, P=0.0026; B, P=0.0231; C, P=0.2; D, P=0.0003, E, P=0.0145; F, P=0.0234). The differences between different therapeutic antibodies are not statistically significant.

FIG. 36. Distinct genetic requirement for irAE and CITE revealed in C57BL/6.Ctla4¹¹⁷″² mice. (A-C) Evaluation of irAE. Female mice (n=5) of given genotypes were treated with either control human IgG (hIg), or anti-PD-1+ Ipilimumab during the perinatal period and evaluated for body weight gain, inflammation and red blood cell anemia at 6 weeks of age. (A) Ipilimumab+ anti-PD-1 combination induced growth retardation in Ctla4^{h/h} but not the Ctla4^{h/m} mice. (B) Except for a modest induction in some mice in the salivary gland, Ipilimumab+ anti-PD-1 did not induce inflammation in internal organs in heterozygous mice. (C) Ipilimumab+ anti-PD-1 did not induce red blood cell anemia in heterozygous mice. (D) Effective tumor rejection induced by Ipilimumab. Tumor bearing Ctla4^{h/h} and Ctla4^{h/m} mice received treatment of either control hIg or Ipilimumab (100 μg/injection×4) on days 7, 10, 13 and 16. The tumor growth was measured every 3 days. Data are mean±S.E.M. and all Data shown were reproduced 2 times. (E) Ipilimumab+ anti-PD-1 did not cause systemic T cell activation in Ctla4^{h/m} mice. Representative FACS profiles depicting the distribution of CD44 and CD62L are shown on the left and summary data are shown on the right. Data in A and D were analyzed by repeated measures two-way ANOVA with Bonferroni's multiple comparison test; whereas those in B, C and E were analyzed by unpaired two-tailed Student's t test.

FIG. 37. irAE and CITE in 6-7 week-old young adult and 10-day old tumor-bearing mice. (A-C) MC38-bearing young male mice (7-week old) were inoculated with MC38 tumor cells and treated with either control hIgG, Ipilimumab, HL12 or HL32 (100 μg/injection×4) on days 7, 10, 13 and 16 after tumor cell challenges. (A) Tumor volumes over time. (B) Serum TNNI3 levels on day 25 after tumor challenge were determined by ELISA. (C) H&E staining show hyalinization and inflammation in the myocardium. Scale bar 100 μm. (D, E) MC38-bearing young male mice (6-week old) were inoculated with MC38 tumor cells and treated with either control hIgG, Ipilimumab or Ipilimumab+ anti-PD-1 (100 μg/injection×4) on days 7, 10, 13 and 16 after tumor cell challenge. (D) Tumor volumes over time. (E) Serum TNNI3 levels on day 25 after tumor challenge were determined by ELISA. (F) 10-day old mice were challenged with MC38 tumors, and immunotherapies were initiated on days 14, 17, 20 and 23 days of age and tumor sizes over time were presented. Data are mean±S.E.M. and analyzed by repeated measures two-way ANOVA with Bonferroni's multiple comparison test. hIg vs Ipilimumab or Ipi+ anti-PD-1, P<0.0001; Ipilimumab vs Ipi+α-PD-1, ns. (G) Combination therapy and monotherapy induced multiple organ inflammations. Representative H&E sections from salivary gland and lung are presented. Scale bar, 100 μm. B and E, data are mean±S.E.M. and statistical significance was analyzed.

FIG. 38A-F. Loss of naïve T cells and increase of effector memory T cells correlate with multiple organ inflammation. Data shown are re-analyses of data presented in FIGS. 16, 25, 28, 29 and 33. Naïve T cells: CD44^{Lo}CD62L^{Hi}; central memory T cells: CD44^{Hi}CD62L^{Hi}; effector memory T cells: CD44^{Hi}CD62L^{Lo}. Correlation coefficient and P-value of linear regression were calculated by Pearson's method.

FIG. 39. Ipilimumab induced modest renal function abnormality in tumor-bearing mice. MC38-bearing mice were treated with 100 μg/injection/mouse for 3 or 4 times on days 7, 10, 13 and 16. Sera were collected on day 18-25 after tumor inoculation. A. The levels of Creatinine and BUN in sera of MC38-bearing hCTLA4-KI mice at day 18-20 (Red: female; blue: male). B. The levels of Creatinine and BUN in sera of MC38-bearing hCTLA4-KI mice (all male) at day 25 after tumor inoculation. Creatinine levels were measured using Creatinine (serum) Colorimetric Assay Kit (Cayman Chemical) or Creatinine (CREA) Kit (RANDOX, Cat No, CR2336). Serum BUN levels were measured using UREA NITROGEN DIRECT kit (Stanbio laboratory). Statistical significance was determined by student's t test.

FIG. 40. Humanization further improves safety of L3D10 based on composite pathology scores. Dark grey dots represent scores of male mice and the lighter grey dots represent female mice used. All scorings were performed double blind. Data are mean±S.E.M., and n=9 mice per group. Statistical significance was determined by one-way ANOVA with Bonferroni's multiple comparison test.

FIG. 41. Distinct mechanisms responsible for irAE and CITE. (A) irAE is caused by inhibiting the conversion of autoreactive T cells into autoreactive Treg, which leads to a polyclonal expansion of autoreactive T cells in the peripheral lymphoid organs. (B) Tumor rejection is achieved by FcR-mediated depletion of Treg in tumor microenvironment and is independent of naïve T cell activation in the peripheral lymphoid organs. Neither irAE nor CITE depends on blockade of B7-CTLA-4 interaction.

FIG. 42. Clinical anti-CTLA-4 mAb Ipilimumab induces cell surface CTLA-4 down-regulation. A-B, 293T cells transfected with human CTLA-4 molecules tagged with orange-fluorescence protein (OFP) were incubated with either control IgG or Ipilimumab (IP) for 4 hrs. (A) The fluorescence of OFP was detected by flow cytometry. B, with or without the present of cycloheximide (CHX), the protein level of CTLA-4 in A was analyzed by Western blot. C, Cell Surface CTLA-4 in A was tested by staining with a commercially available anti-CTLA-4 mAbs (BNI3), which has strong binding to cell surface CTLA-4 even in the presence of saturating doses of Ipilimumab. D, Plasma membrane proteins in A were isolated and the surface CTLA-4 was detected by Western blot. E, CHO stable cell lines expressing human CTLA-4 were treated with either control IgG or Ipilimumab (IP) for 4 hrs. The protein level of CTLA-4 was analyzed by Western blot. F, Cell Surface CTLA-4 in E was tested by flow cytometry. G, Plasma membrane proteins in E were isolated and the surface CTLA-4 was detected by Western blot. H, MC38 mouse colon cancer model was induced in C57BL/6 Ctla-4^{h/h}-KI mice. Tumor cells were isolated by collagenase digestion and treated with either control IgG or Ipilimumab (IP) for 4 hrs in vitro. Surface and intracellular CTLA-4 of tumor-infiltrating Tregs was tested by flow cytometry. I, MC38 bearing-Ctla4^{h/h} mice were i.p. treated with 100 μg of either control IgG or Ipilimumab (IP) for 16 hrs on day 14 after tumor inoculation. Surface and intracellular CTLA-4 of tumor-infiltrating Tregs was tested by flow cytometry. Results in C and H are triplicates (mean±SEM). Data in I are mean±SEM (n=8). *p<0.05, **p<0.01. Unpaired two-tailed Student's t test.

FIG. 43. Ipilimumab induces cell surface CTLA-4 down-regulation in immunotherapy-related adverse effect (irAE) model and in activated human Tregs. A, CTLA-4^{h/h}-KI neonatal mice (n=5) were i.p. treated with anti-PD-1 (100 ug) for either 24 hrs or 48 hrs. After that, mice were further i.p. treated with 100 μg of control IgG or Ipilimumab for 4 hrs. Surface and intracellular CTLA-4 of lung and spleen Tregs were evaluated by flow cytometry. B-C, Human PBMCs from healthy donors' blood were stimulated by anti-CD3/anti-CD28 for 2 days and treated with either control IgG or Ipilimumab (IP) for 4 hrs. Surface CTLA-4 of CD4⁺CD25⁺Foxp3⁺ Tregs and CD4⁺CD25⁺Foxp3⁻ non-Tregs was measured by flow cytometry (B). Analysis of CD4⁺CD25⁺Foxp3⁺ Tregs from four health donors has been shown in (C). Data are mean±SEM. *p<0.05, **p<0.01, #p<0.001. Unpaired two-tailed Student's t test.

FIG. 44. The antibody-induced down-regulation of surface CTLA-4 causes immunotherapy-related adverse effect (irAE). A, C57BL/6 Ctla4^{h/h} neonatal mice were treated, respectively, with control human IgG+ anti-PD-1, Tremelimumab (IgG1)+ anti-PD-1, or HL12+ anti-PD-1 at a dose of 100 μg/mouse/injection on age of days 10, 13, 16 and 19. Weight gains of different treatments are shown. One mouse from the Tremelimumab (IgG1) plus anti-PD1 treated group was excluded from analysis for death on day 18 age with serious growth retardation. Data shown are means and SEM of % weight gain following the first injection. B, The CBC analysis of blood from mice in A was performed on day 41 after birth. Data of blood hematocrit (HCT), total hemoglobin (Hb) and Mean Corpuscular Volume (MCV) are shown. C, MC38 bearing-Ctla4^{h/h} mice (n=5) were i.p. treated with either control Ig (100 μg) or Tremelimumab (IgG1) (1 μg, 30 μg or 100 μg) on days 7, 10, 13 and 16 after tumor inoculation. Tumor volumes shown were means and SEM of % weight gain following the first injection. D, 293T cells transfected with hCTLA-4 were incubated with either control IgG, Ipilimumab, Tremelimumab (IgG1), HL12 or HL32 for 4 hrs. The protein level of CTLA-4 was analyzed by Western blot. E, CHO stable cell lines expressing hCTLA-4 were treated with Ipilimumab, Tremelimumab (IgG1), HL12 or HL32 for 2 hrs at 4/37° C. After washing out the unbound antibodies, surface CTLA-4 was detected by anti-hIgG (H+L)-alex488 for half an hour at 4° C. and analyzed by flow cytometry. After normalization by subtracting the alex488 fluorescence at 4° C., the fluorescence intensity of surface CTLA-4 shown are triplicates (mean+SEM) *P<0.05, unpaired two-tailed t-test. F, 293T cells transfected with hCTLA-4 were incubated with either control IgG Ipilimumab or HL12 for 4 hrs. Plasma membrane proteins were isolated and the surface CTLA-4 was detected by Western blot. G, Ctla-4^{h/h}-KI neonatal mice (n=6) were i.p. treated with anti-PD-1 (100 ug). 24 hrs after that, mice were further i.p. treated with 100 μg of either control IgG, Ipilimumab or HL12 for 4 hrs. Surface and intracellular CTLA-4 of lung and spleen Tregs were evaluated by flow cytometry. Since HL12 blocks the binding of BIN3 to CTLA-4, saturating doses of HL12 were added before CTLA-4 staining by BNI3 clone when comparing HL12 group with control group. H, HL12 treatments in G were evaluated by staining cells with another commercial anti-CTLA-4 mAbs (eBio20A), which did not block the binding of HL12 to CTLA-4. I, Human PBMCs from healthy donors' blood were stimulated by anti-CD3/anti-CD28 for 2 days and treated with either control IgG, Ipilimumab (IP) or HL12 for 4 hrs. Surface CTLA-4 of CD4⁺CD25⁺Foxp3⁺ Tregs was measured by flow cytometry. Anti-CTLA-4 mAbs (BNI3) were used for comparing control and Ipilimumab groups while anti-CTLA-4 mAbs (eBio20A) were used for HL12 group. Data in G and H are mean±SEM. Results in I are triplicates (mean±SEM). *p<0.05, **p<0.01, #p<0.001. Unpaired two-tailed Student's t test.

FIG. 45. Anti-CTLA-4 mAbs regulate surface CTLA-4 through lysosome-mediated degradation. A, Surface CTLA-4 on CHO stable cell lines expressing hCTLA-4 was labeled with either Ipilimumab-Alex488 or HL12-Alex488 at 4° C. and transferred to 37° C. for 30 min. Representative confocal images of antibody-labeled surface CTLA-4 are shown. B, Surface CTLA-4 in A was stained with lyso-tracker and co-localization between surface CTLA-4 and lysosomes is shown by confocal images (Green: surface CTLA-4; Pink: Lysosomes; White: Merge). C, Time-span of cell surface CTLA-4 localization after Ipilimumab and HL12 induced CTLA-4 internalization in B has been shown by representative confocal images. D, with or without pre-treatment of lysosome inhibitor chloroquine (CQ), 293T cells transfected with hCTLA-4 were incubated with either control IgG or Ipilimumab for 4 hrs. The protein level of CTLA-4 was analyzed by Western blot.

FIG. 46. Anti-CTLA-4 mAbs, which reserve higher binding affinity during endosome-lysosome transportation, facilitate lysosomal degradation of surface CTLA-4. A, His-hCTLA-4 (0.5 μg/ml) was coated in ELISA plates and different anti-CTLA4-mAbs were added at 10 μg/ml in the buffer at different pH range from pH 4.0 to 7.0. Antibodies binding with CTLA-4 were measured by ELISA. B. Comparison of limiting doses of different anti-CTLA-4 antibodies at pH4.5, 5.5 and 7.0. His-hCTLA-4 (0.5 μg/ml) was coated in ELISA plates and varying doses of anti-CTLA4-mAbs were measured for their binding to CTLA-4. Note that Ipilimumab and Tremelimumab exhibit essentially identical dose response at pH7.0 and pH5.5. The amounts of antibodies needed at pH5.5 to achieve 50% maximal pH7.0 binding (IC50) were essentially the same at those needed at pH7.0. The IC50 at pH4.5 was increased by approximately 50-250%. In contrast, HL12 and HL32 exhibit more than 10-fold reduction when binding at pH5.5 was compared with that at pH7.0, based on increase of IC50. The reduction of IC at pH4.5 is greater than 100-fold reduction was observed when their binding at pH 4.5 was compared to pH7.0, again based on increase of IC50. C, His-hCTLA-4 (0.5 μg/ml) was coated and different anti-CTLA4-mAbs were added at 10 μg/ml at pH 7.0. After extra antibodies were washed away, binding of CTLA-4 was detected followed by 2 h incubation at lower pH buffer (pH4.5, 5.5, and 6). Data in A-C are means of duplicate optical density at 450 nm. D, Surface CTLA-4 was labeled with anti-CTLA-4 mAbs at 4° C. for 30 min then transferred to 37° C. for 1 h. Antibody-bound CTLA-4 was captured by protein-G beads and tested by Western blot. E, Surface CTLA-4 in D was pre-treated with or without CQ, which neutralized endosomal-lysosomal pH, for 30 min. Antibody-bound CTLA-4 was captured by protein-G beads and tested by Western blot.

FIG. 47. Internalized CTLA-4 triggered by HL12 recycles back to cell surface and prevents anti-CTLA-4-induced irAE. A, 293T cells transfected with human Rab11 tagged with dsRed were incubated with either Ipilimumab-Alex488 or HL12-Alex488 at 4° C. for 30 min. and transferred to 37° C. for 1 h. Representative confocal images of co-localization between surface CTLA-4 and Rab11 are shown (Green: surface CTLA-4; Red: Rab11; Blue: Nuclei; Yellow: Merge of CTLA-4 and Rab11). B, 293T cells transfected with hCTLA-4-GFP were incubated with either control IgG, Ipilimumab or HL12 for 4 hrs. Representative confocal images of surface CTLA-4 are shown. C, Model depicting the distinct regulation by anti-CTLA-4 mAbs responsible for antibody-induced immunotherapy-related adverse effect (irAE).

FIG. 48. pH-sensitive anti-CTLA-4 antibodies are more efficient in induction of Treg in tumor microenvironment. Ctla4^{h/h} mice that bore MC38 tumors received either control hIgG, Ipilimumab, HL32 or HL12 (100 μg/mouse) on day 14 after tumor inoculation. % Treg cells among CD4 T cells in the tumor were determined were determined by flow cytometry of single cell suspensions of tumors harvested at 16 hours after antibody treatment. Note that two pH sensitive antibodies, HL12 and HL32 caused rapid Treg depletion at 24 hours after antibody treatment (P<0.0001). In contrast, while Ipilimumab can cause Treg depletion after repeated dosing (FIG. 21), it is largely ineffective at 24 hours after single dosing.

FIG. 49. pH-sensitive anti-CTLA-4 antibodies are more efficient in inducing rejection of large established tumors. Ctla4^{h/h} mice that bore MC38 tumors received either control hIgG, ipilimumab, Tremelimumab (IgG1) (TremeIgG1), HL32 or HL12 (30 μg/mouse) on days 17 and 20 after tumor inoculation. Tumor sizes were measured using a caliber

### DETAILED DESCRIPTION

### 1. Definitions

As used herein, the term “antibody” refers to an immunoglobulin molecule that possesses a “variable region” antigen recognition site. The term “variable region” refers to a domain of the immunoglobulin that is distinct from a domains broadly shared by antibodies (such as an antibody Fc domain). The variable region comprises a “hypervariable region” whose residues are responsible for antigen binding. The hypervariable region comprises amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; ref. 44) and may comprise those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Ref. 45). “Framework Region” or “FR” residues are those variable domain residues other than the hypervariable region residues as herein defined. An antibody disclosed herein may be a monoclonal antibody, multi-specific antibody, human antibody, humanized antibody, synthetic antibody, chimeric antibody, camelized antibody, single chain antibody, disulfide-linked Fv (sdFv), intrabody, or an anti-idiotypic (anti-Id) antibody (including, e.g., anti-Id and anti-anti-Id antibodies to antibodies of the invention). In particular, the antibody may be an immunoglobulin molecule, such as IgG, IgE, IgM, IgD, IgA or IgY, or be of a class, such as IgG₁, IgG₂, IgG₃, IgG₄, IgA₁ or IgA₂, or of a subclass.

As used herein, the term “antigen binding fragment” of an antibody refers to one or more portions of an antibody that contain the antibody's Complementarity Determining Regions (“CDRs”) and optionally the framework residues that comprise the antibody's “variable region” antigen recognition site, and exhibit an ability to immunospecifically bind antigen. Such fragments include Fab′, F(ab′).sub.2, Fv, single chain (ScFv), and mutants thereof, naturally occurring variants, and fusion proteins comprising the antibody's “variable region” antigen recognition site and a heterologous protein (e.g., a toxin, an antigen recognition site for a different antigen, an enzyme, a receptor or receptor ligand, etc.). As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

Human, chimeric or humanized antibodies are particularly preferred for in vivo use in humans, however, murine antibodies or antibodies of other species may be advantageously employed for many uses (for example, in vitro or in situ detection assays, acute in vivo use, etc.).

A “chimeric antibody” is a molecule in which different portions of the antibody are derived from different immunoglobulin molecules such as antibodies having a variable region derived from a non-human antibody and a human immunoglobulin constant region. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; 46-48), and chain shuffling (U.S. Pat. No. 5,565,332), the contents of all of which are incorporated herein by reference.

The invention particularly concerns “humanized antibodies.” As used herein, the term “humanized antibody” refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. A humanized antibody is an antibody comprising a humanized light chain and a humanized heavy chain immunoglobulin. For example, a humanized antibody would not encompass a typical chimeric antibody, because, e.g., the entire variable region of a chimeric antibody is non-human. The donor antibody is referred to as being “humanized,” by the process of “humanization,” because the resultant humanized antibody is expected to bind to the same antigen as the donor antibody that provides the CDRs. Humanized antibodies may be human immunoglobulins (recipient antibody) in which hypervariable region residues of the recipient are replaced by hypervariable region residues from a non-human species (donor antibody) such as mouse, rat, rabbit or a non-human primate having the desired specificity, affinity, and capacity. In some instances, Framework Region (FR) residues of the human immunoglobulin are replaced by corresponding non-human residues. Furthermore, humanized antibodies may comprise residues which are not found in the recipient antibody or in the donor antibody. These modifications may further refine antibody performance. The humanized antibody may comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody may optionally also comprise at least a portion of an immunoglobulin constant region (Fc), which may be that of a human immunoglobulin that immunospecifically binds to an FcγRIIB polypeptide, that has been altered by the introduction of one or more amino acid residue substitutions, deletions or additions (i.e., mutations).

### 2. Anti-CTLA4 Antibody Compositions

An antibody against human CTLA-4 protein, Ipilimumab, has been shown to increase survival of cancer patients, either as the only immunotherapeutic agent or in combination with another therapeutic agent such as an anti-PD-1 antibody. However, the CITE is associated with significant immune-related significant adverse effects (irAEs). There is a great need to develop novel anti-CTLA-4 antibodies to achieve better therapeutic effects or fewer autoimmune adverse effects. The inventors have discovered anti-CTLA-4 antibodies that, surprisingly, can be used to induce cancer rejection without significant autoimmune adverse effects associated with immunotherapy.

Provided herein are antibodies and antigen-binding fragments thereof, and compositions comprising the foregoing. The composition may be a pharmaceutical composition. The antibody may be an anti-CTLA-4 antibody. The antibody may be a monoclonal antibody, a human antibody, a chimeric antibody or a humanized antibody. The antibody may also be monospecific, bispecific, trispecific, or multispecific. The antibody may be detectably labeled, and may comprise a conjugated toxin, drug, receptor, enzyme, or receptor ligand.

Also provided herein is an antigen-binding fragment of an antibody that immunospecifically binds to CTLA-4, and in particular human CTLA-4, which may be expressed on the surface of a live cell at an endogenous or transfected concentration. The antigen-binding fragment may bind to CTLA-4, and the live cell may be a T cell.

In a particular embodiment, the anti-CTLA-4 antibody may efficiently induce Treg depletion and Fc receptor-dependent tumor rejection. In a preferred embodiment, to increase the anti-tumor activity, CTLA-4 targeting agents will selectively deplete Tregs in the tumor microenvironment. In a particular embodiment, the anti-CTLA-4 mAbs have increased Fc mediated Treg depleting activity. Treg depletion can occur by Fc mediated effector function such as antibody-dependent cell-mediated cytotoxicity (ADCC) or antibody-dependent cell-mediated phagocytosis (ADCP). The Fc mediated effector function can be introduced or enhanced by any method known in the art. In one example the antibody is an IgG1 isotype, which has increased effector function compared to other isotypes. The Fc mediated effector function can be further enhanced by mutation of the amino acid sequence of the Fc domain. For example, three mutations (S298A, E333A and K334A) can be introduced into the CH region of the Fc domain to increase ADCC activity. Antibodies used for ADCC mediated activity usually require some kind of modification in order to enhance their ADCC activity. There are a number of technologies available for this which typically involves engineering the antibody so that the oligosaccharides in the Fc region of the antibody do not have any fucose sugar units, which improves binding to the FcγIIIa receptor. When antibodies are afucosylated the effect is to increase antibody-dependent cellular cytotoxicity (ADCC). For example, Biowa's POTELLIGENT® technology uses a FUT8 gene knockout CHO cell line to produce 100% afucosylated antibodies. FUT8 is the only gene coding a 1,6-Fucosyltransferase which catalyzes the transfer of Fucose from GDP-Fucose to GlcNAc in a 1,6-linkage of complex-type oligosaccharide. Probiogen has developed a CHO line that is engineered to produce lower levels of fucosylated glycans on MAbs, although not through FUT knockout. Probiogen's system introduces a bacterial enzyme that redirects the de-novo fucose synthesis pathway towards a sugar-nucleotide that cannot be metabolized by the cell. As an alternative approach, Seattle Genetics has a proprietary feed system which will produce lower levels of fucosylated glycans on MAbs produced in CHO (and perhaps other) cell lines. Xencor has developed an XmAb Fc domain technology is designed to improve the immune system's elimination of tumor and other pathologic cells. This Fc domain has two amino acid changes, resulting in a 40-fold greater affinity for FcγRIIIa. It also increases affinity for FcγRIIa, with potential for recruitment of other effector cells such as macrophages, which play a role in immunity by engulfing and digesting foreign material.

In another embodiment, the anti-CTLA-4 antibody may not confer complete CTLA-4 occupation (i.e. non-blocking or not completely blocking), systemic T cell activation or preferential expansion of self-reactive T cells.

In another embodiment, the anti-CTLA-4 antibody has weak binding affinity to CTLA-4 at low pH and will dissociate from CTLA-4 during antibody-induced internalization, allowing released CTLA-4 to recycle back to the cell surface and maintain the function of CTLA-4 as a negative regulator of immune response. Such an antibody may show >3-fold reduction in binding at pH5.5 when compared to that at pH7.0, based on increase of doses of antibodies needed at late endosomal pH5.5 to achieve 50% maximal binding at pH7.0. At lysosomal pH4.5, such reduction reaches 10-fold or more. Preferably, reduction at pH5.5 and pH4.5 would be greater than 10 and 100-fold respectively,

In another embodiment, the anti-CTLA-4 antibody has reduced binding affinity to sCTLA-4 so that sCTLA-4 in circulation may maintain its function as a negative regulator of immune response.

In a preferred embodiment, the anti-CTLA-4 antibody has two or more of these properties. Specifically, the anti-CTLA-4 antibody will selectively deplete Tregs in the tumor microenvironment without antagonizing (i.e. depleting or blocking) the function of membrane bound or soluble CTLA-4 so that it may maintain the function of negative regulator of immune response.

### 3. Methods of Designing and Selecting Antibodies

Further provided herein are the design and/or selection of new anti-CTLA-4 antibodies, and ways to engineer antibodies to enhance the anti-tumor efficacy and/or toxicity profile of existing anti-CTLA-4 antibodies, by incorporating the functional characteristics or attributes of the antibodies described herein. Specifically, provided are methods of increasing the Treg depleting activity of the anti-CTLA-4 antibody to increase CITE, and reducing the endosome trafficking and destruction of antibody bound CTLA-4, to improve the toxicity profile by allowing CTLA-4 to recycle to the cell surface. In a most preferred embodiment, the anti-CTLA-4 antibody is designed or engineered to improve both the Treg depleting activity and the CTLA-4 recycling activity. As anti-human CTLA-4 antibodies tend to not cross react with CTLA-4 from other species, such as mice, is understood that such testing must use a human CTLA4 system such as human cells, cells transfected with human CTLA-4, or a transgenic animal model that expresses human CTLA-4 such as the human CTLA-4 knockin mouse described herein. In one embodiment, antibodies are designed to enhance the depletion of Tregs within the tumor environment. Such antibodies can be tested or selected using any one of the in vitro or in vivo methods described herein. For example, human CTLA-4 knockin mice are injected with a tumor cell line along with the anti-CTLA-4 antibodies, and at a later time point the tumor infiltrating Tregs are removed and counted, and compared to a negative or positive control.

In another embodiment, antibodies are designed to reduce their ability to induce toxicity, particularly irAEs. This is best tested in vivo using a human CTLA-4 expressing animal model. In a preferred embodiment, the anti-CTLA-4 antibodies, either alone or in combination, are administered to mice at the perinatal or neonatal stage to determine their ability to induce irAEs. Readouts for toxicity or irAEs include reduced body weight gain, hematology (CBC), histopathology, and survival.

As demonstrated herein, as a surrogate or their ability to reduce irAEs, the anti-CTLA-4 antibodies can be assayed for their ability to release CTLA-4 at endosomal (acidic) pH. In one embodiment, this can be determined in vitro by assaying the ability to bind CTLA-4 molecules over a pH range. More specifically, the anti-CTLA-4 antibodies can be added at limiting doses to determine the amounts needed at low pH to achieve 50% of maximal binding achieved at pH 7.0. In another embodiment, this can be assayed using cells in vitro whereby the internalization and intracellular localization and trafficking of cell surface CTLA-4 following anti-CTLA-4 engagement is tracked. In one embodiment, the localization of the CTLA-4 protein can be compared to an endosomal marker (e.g. LysoTracker) wherein co-localization with the endosomal marker indicates endosomal degradation and lack of recycling, which in turn correlates with the ability to induce irAEs. In another embodiment, the ability of the internalized CTLA-4 to recycle to the cell surface can be assayed using a fluorescent-CTLA-4 protein, wherein recycling back to the cell surface correlates with the ability to reduce irAEs. In yet another embodiment, the ability of the internalized CTLA-4 to recycle to the cell surface and reduce irAEs can be assayed by co-localization with a marker for recycling endosomes, such as Rab11.

In another embodiment, antibodies are designed or selected for reduced binding to or blocking of soluble CTLA-4 (sCTLA-4). This can be tested in vitro by testing the ability of a soluble CTLA-4 molecule, such as CTLA-4-Fc, to bind to its natural ligand (B7-1 or B7-2) or another anti-CTLA-4 molecule immobilized on a plate or cell surface. In a preferred embodiment, the soluble CTLA-4 molecule is labeled so that its presence after binding can be detected.

### 4. Methods of Treatment

The invention further concerns the use of the antibody compositions described herein for the upregulation of immune responses. Up-modulation of the immune system is particularly desirable in the treatment of cancers and chronic infections, and thus the present invention has utility in the treatment of such disorders. As used herein, the term “cancer” refers to a neoplasm or tumor resulting from abnormal uncontrolled growth of cells. “Cancer” explicitly includes leukemias and lymphomas. The term “cancer” also refers to a disease involving cells that have the potential to metastasize to distal sites.

Accordingly, the methods and compositions of the invention may also be useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include, but are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other specific embodiments, sarcoma, melanoma, or leukemia is treated or prevented by the methods and compositions of the invention.

In another embodiment of the invention, the antibody compositions and antigen binding fragments thereof can be used with another anti-tumor therapy, which may be selected from but not limited to, current standard and experimental chemotherapies, hormonal therapies, biological therapies, immunotherapies, radiation therapies, or surgery. In some embodiments, the molecules of the invention may be administered in combination with a therapeutically or prophylactically effective amount of one or more agents, therapeutic antibodies or other agents known to those skilled in the art for the treatment or prevention of cancer, autoimmune disease, infectious disease or intoxication. Such agents include for example, any of the above-discussed biological response modifiers, cytotoxins, antimetabolites, alkylating agents, antibiotics, anti-mitotic agents, or immunotherapeutics.

In preferred embodiment of the invention, the antibody compositions and antigen binding fragments thereof can be used with another anti-tumor immunotherapy. In such an embodiment, the antibody of the invention or antigen binding fragment thereof is administered in combination with a molecule that disrupts or enhances alternative immunomodulatory pathways (such as TIM3, TIM4, OX40, CD40, GITR, 4-1-BB, B7-H1, PD-1, B7-H3, B7-H4, LIGHT, BTLA, ICOS, CD27 or LAG3) or modulates the activity of effecter molecules such as cytokines (e.g., IL-4, IL-7, IL-10, IL-12, IL-15, IL-17, GF-beta, IFNg, Flt3, BLys) and chemokines (e.g., CCL21) in order to enhance the immunomodulatory effects. Specific embodiments include a bi-specific antibody comprising an anti-CTLA4 antibody described herein or antigen binding fragment thereof, in combination with anti-PD-1 (pembrolizumab (Keytruda) or Nivolumab (Opdivo)), anti-B7-H1 (atezolizumab (Tecentriq) or Durvalumab (Imfinzi), anti-B7-H3, anti-B7-H4, anti-LIGHT, anti-LAG3, anti-TIM3, anti-TIM4 anti-CD40, anti-OX40, anti-GITR, anti-BTLA, anti-CD27, anti-ICOS or anti-4-1BB. In yet another embodiment, an antibody of the invention or antigen binding fragment thereof is administered in combination with a molecule that activates different stages or aspects of the immune response in order to achieve a broader immune response, such as MO inhibitors. In more preferred embodiment, the antibody compositions and antigen binding fragments thereof are combined with anti-PD-1 or anti-4-1BB antibodies, without exacerbating autoimmune side effects.

Another embodiment of the invention includes a bi-specific antibody that comprises an antibody that binds to CTLA4 bridged to an antibody that binds another immune stimulating molecule. Specific embodiments include a bi-specific antibody comprising the anti-CTLA4 antibody compositions described herein and anti-PD-1, anti-B7-H1, anti-B7-H3, anti-B7-H4, anti-LIGHT, anti-LAG3, anti-TIM3, anti-TIM4 anti-CD40, anti-OX40, anti-GITR, anti-BTLA, anti-CD27, anti-ICOS or anti-4-1BB. The invention further concerns of use of such antibodies for the treatment of cancer.

### 5. Production

The anti-CTLA4 antibodies described herein and antigen binding fragments thereof may be prepared using a eukaryotic expression system. The expression system may entail expression from a vector in mammalian cells, such as Chinese Hamster Ovary (CHO) cells. The system may also be a viral vector, such as a replication-defective retroviral vector that may be used to infect eukaryotic cells. The antibodies may also be produced from a stable cell line that expresses the antibody from a vector or a portion of a vector that has been integrated into the cellular genome. The stable cell line may express the antibody from an integrated replication-defective retroviral vector. The expression system may be GPEx™.

The anti-CTLA4 antibodies described herein and antigen binding fragments thereof can be purified using, for example, chromatographic methods such as affinity chromatography, ion exchange chromatography, hydrophobic interaction chromatography, DEAE ion exchange, gel filtration, and hydroxylapatite chromatography. In some embodiments, antibodies can be engineered to contain an additional domain containing an amino acid sequence that allows the polypeptides to be captured onto an affinity matrix. For example, the antibodies described herein comprising the Fc region of an immunoglobulin domain can be isolated from cell culture supernatant or a cytoplasmic extract using a protein A or protein G column. In addition, a tag such as c-myc, hemagglutinin, polyhistidine, or Flag™ (Kodak) can be used to aid antibody purification. Such tags can be inserted anywhere within the polypeptide sequence, including at either the carboxyl or amino terminus. Other fusions that can be useful include enzymes that aid in the detection of the polypeptide, such as alkaline phosphatase. Immuno-affinity chromatography also can be used to purify polypeptides.

### 6. Pharmaceutical Compositions

The invention further concerns a pharmaceutical composition comprising a therapeutically effective amount of any of the above-described anti-CTLA4 antibody compositions or antigen binding fragments thereof, and a physiologically acceptable carrier or excipient. Preferably, compositions of the invention comprise a prophylactically or therapeutically effective amount of the anti-CTLA4 antibody or its antigen binding fragment and a pharmaceutically acceptable carrier

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers may be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, trehalose, gelatin, malt, rice, flour, chalk, silica gel, sodium stearate, glycerol monostearate, talc, sodium chloride, dried skim milk, glycerol, propylene, glycol, water, ethanol and the like. The composition, if desired, may also contain minor amounts of wetting or emulsifying agents, such as Poloxamer or polysorbate, or pH buffering agents. These compositions may take the form of solutions, suspensions, emulsion, tablets, pills, capsules, powders, sustained-release formulations and the like.

Generally, the ingredients of compositions of the invention may be supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline may be provided so that the ingredients may be mixed prior to administration.

The compositions of the invention may be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The anti-CTLA-4 antibody compositions described herein, or antigen binding fragments thereof, may also be formulated for lyophilization to allow long term storage, particularly at room temperature. Lyophilized formulations are particularly useful for subcutaneous administration.

### 7. Methods of Administration

Methods of administering the compositions described herein include, but are not limited to, parenteral administration (e.g., intradermal, intramuscular, intraperitoneal, intravenous and subcutaneous), epidural, and mucosal (e.g., intranasal and oral routes). In a specific embodiment, the antibodies of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local.

### EXAMPLES

### Example 1

### Anti-CTLA-4 mAbs Cause Tumor Rejection by Mechanisms that are Independent of Checkpoint Blockade but Dependent on Host Fc Receptor

Materials and Methods

Animals

CTLA4 humanized mice that express the CTLA-4 protein with 100% identity to human CTLA-4 protein under the control of endogenous mouse Ctla4 locus have been described [38]. The homozygous knock-in mice (Ctla4^{h/h}) were backcrossed to C57BL/6 background for at least 10 generations. Heterozygous mice (Ctla4^{h/m}) were produced by crossing the Ctla4^{h/h} mice with wild type (WT) BALB/c or C57BL/6 mice. WT C57BL/6 mice were purchased from Charles River Laboratories. Human cord blood CD34⁺ stem cell reconstituted NSG™ mice were obtained from the Jackson Laboratories (Bar Harbor, Me.). All animals (both female and male, 6-16 weeks old, age-matched in each experiment) were included in the analysis, and no blinding or randomization was used, except that mice were randomly assigned to each group. All mice were maintained at the Research Animal Facility of Children's Research Institute at the Children's National Medical Center. All studies involving mice were approved by the Institutional Animal Care and Use Committee.

Cell Culture

No cell lines used in this study were listed in the database of cross-contaminated or misidentified cell lines suggested by International Cell Line Authentication Committee (ICLAC). CHO cells and L929 cells transfected with mouse or human B7-1 or B7-2 have been described previously [20, 29]. B7-1-transfected J558 cells [22] P815 cells transfected with B7-H2-GFP [50] have been described previously. Murine colon tumor cell line MC38 was described previously [5]. Melanoma cell line B16-F10 (ATCC® CRL-6475™) and HEK 293T cells (ATCC® CRL-11268™) was originally purchased from ATCC (Manassas, Va., USA). After receiving from vendors, cell passages were kept minimal before in vivo testing. All cell lines were incubated at 37° C. and were maintained in an atmosphere containing 5% CO₂. Cells were grown in DMEM (Dulbecco's Modified Eagle Medium, Gibco) supplemented with 10% FBS (Hyclone), 100 units/mL of penicillin and 100 μg/mL of streptomycin (Gibco).

Antibodies

Mouse anti-human CTLA-4 mAb L3D10 has been described [15]. Anti-CTLA-4 mAb L3D10 used in the study was a chimera antibody consisting of human IgG1 Fc and the variable regions of L3D10. Recombinant WT (M1) and mutated (M17, M17-4) hCTLA-4 proteins, as well as recombinant antibodies including parental and fully humanized L3D10 (clones HL12 and HL32) were produced by Lakepharma, Inc (Belmont, Calif., USA). Recombinant Ipilimumab with amino acid sequence disclosed in WC500109302 and http://www.drugbank.ca/drugs/DB06186 was provided by Alphamab Inc. (Suzhou, Jiangsu, China), or Lakepharma Inc (San Francisco, Calif., USA) of leftover clinical samples. Human IgG-Fc (No azide) was bulk ordered from Athens Research and Technology (Athens, Ga., USA). Anti-mouse CD16/32 mAb 2.4G2, anti-mouse B7-1mAb 1G10, anti-mouse B7-2 mAb GL1, anti-mouse Ctla-4 mAbs 9D9 and 9H10, control hamster IgG, control mouse IgG2b MPC-11, and human CTLA-4-Fc were purchased from Bio-X-Cell Inc. (West Lebanon, N H, USA). Purified hamster anti-mouse Ctla-4 mAb 4F10 was purchased from BD Biosciences (San Jose, Calif., USA). Purified and biotinylated hamster IgG isotype control antibodies used for in vitro blocking assays were purchased from eBioscience (San Diego, Calif., USA). Fusion proteins for human B7-1-Fc, B7-2-Fc, and polyhistidine tagged human CTLA-4 were purchased from Sino Biological Inc. (Beijing, China). Recombinant mouse Ctla-4Fc protein was purchased from BioLegend (San Diego, Calif., USA). Biotinylation was completed by conjugating EZ-Link Sulfo-NHS-LC-Biotin (Thermo Scientific) to desired proteins according to the manufacturer's instructions. Alexa Fluor 488-conjugated goat anti-human IgG (H+L) cross-adsorbed secondary antibody was purchased from ThermoFisher Scientific, USA. The levels of cytokines IL-4, IL-6 and IL-10 were evaluated by Cytometric Beads Array (BD Biosciences, Catalogue number 560485) following the manufacture's protocol. SIY peptide was purchased from MBL International Corporation (Woburn, Mass., USA), and SIY-specific CD8 T cells were detected by H-2K^{b} tetramer SIYRYYGL-PE (MBL Code #TS-M008-1). H-2K^{b} tetramer OVA (SIINFEKL)-PE provided by NIH (#31074) was used as negative control for flow stainings.

Assays for In Vitro and In Vivo Blockade of B7-CTLA-4 Interaction

Three assays were employed to assess the blocking activities of anti-CTLA-4 mAbs. First, plates were coated with either CTLA-4-Fc or their ligand, B7-1. Biotinylated fusion proteins were used in soluble phase in the binding assay, with the amounts of protein bound measured by horse-radish peroxidase (HRP)-conjugated avidin (Pierce High Sensitivity NeutrAvidin-HRP, Thermo Scientific Inc.). Proteins were coated in bicarbonate buffer (0.1M) at 4° C. and the binding assays were performed at room temperature.

Second, flow cytometry was used to detect binding of biotinylated fusion protein to CHO cells transfected to express mouse or human B7-1 and B7-2 on the cell surface. In each assay consisting of 105 μl PBS solution, 1.2×10⁵ CHO cells were incubated with 200 ng biotinylated human or mouse CTLA-4 protein, along with varying doses of anti-human or mouse CTLA-4 mAbs or control IgG, for 30 min at room temperature. The amounts of bound receptors were measured using phycoethrythorin (PE)-conjugated streptavidin purchased from BioLegend (San Diego, Calif., USA). Flow cytometry was performed using FACS CantoII (BD Biosciences), and data were analyzed by FlowJo (Tree Star Inc.).

Third, the up-regulation of B7-1 and B7-2 by anti-CTLA-4 mAbs was used as the readout for blockade of B7-1-CTLA-4 and B7-2-CTLA-4 interaction. Briefly, age and gender-matched mice received 500 μg of antibodies or their controls intraperitoneally. At 24 hours after injection, mice were sacrificed and their spleen cells were stained with antibodies against CD11c (clone N418), CD11b (clone M1/70), B7-1 (clone 16-10-A1) and B7-2 (clone PO3.1) and isotype control Abs purchased from eBioscience Inc (San Jose, Calif., USA). NSG™ mice reconstituted with human CD34⁺ cord blood cells received the same doses of antibodies. The spleens were meshed between two frosted microscope slides, and then incubated for 20 min at 37° C. in 5 ml buffer containing 100 μg/ml Collagenase Type IV and 5 U/ml DNase I. A cell suspension was prepared by gently pushing the digested nodes through a cell strainer, and stained with the antibodies specific for the following markers: hB7-1, clone 2D10 (Biolegend Cat. No 305208); hB7-2: clone IT2.2 (BioLegend, Cat No. 305438); hCD11c, clone 3.9; BioLegend Cat No. 301614); HLA-DR, clone L243 (BioLegend Cat. No. 307616); hCD45, clone HI30 (BioLegend, Cat. No. 304029).

Transendocytosis Assay and Cell-Cell Interaction Assay

Plasmids with GFP (C-GFPSpark tag)-tagged human B7-2/B7-1 and OFP (C-OFPSpark tag)-tagged human CTLA-4 cDNA were purchased from Sino Biological Inc. (Beijing, China) and used to establish stable CHO cell lines expressing either molecule. To measure inhibition of transendocytosis by anti-CTLA-4 mAbs, the Fab fragments were prepared with the Pierce™ Fab Preparation Kit (Thermo Scientific, USA) following the manufacturer's instruction. Given doses of the Fab or control hIgG-Fc proteins were added to GFP-tagged B7-2 expressing CHO cells immediately prior to their co-culturing with OFP-tagged CTLA-4 expressing CHO cells at 37° C. for 4 hours.

Plasmids encoding OFP-tagged human CTLA-4 or human CTLA4^{Y201V} cDNA was used to establish stable HEK293T cell lines. After overnight suspension culturing in 15 mL centrifuge tubes, B7-GFP tagged CHO cells and CTLA4^{Y201V}-OFP tagged HEK293T cells were co-incubated at an approximately 2:1 ratio at 4° C. for 2 hours. Given doses of the Fab or control hIgG-Fc proteins were added to the mixed cells immediately prior to their co-culturing. For both transendocytosis and cell-cell interaction assays, 1×10⁵ B7-GFP tagged CHO cells were used in each single test. The amounts of transendocytosis and cell-cell interaction were determined by flow cytometry based on acquisition of GFP signal from the B7-GFP-transfected CHO cells by CTLA-4-OFP-transfected CHO cells or CTLA4^{Y201V}-OFP transfected HEK293T cells.

The following formula is used for the calculation of both assays:

% transendocytosis or Cell-cell interaction

=(GFP⁺OFP⁺%)/(GFP⁺OFP⁺%+GFP⁻OFP⁺%)

Kinetics of B7-CTLA-4 Interaction

Binding experiments were performed on Octet Red96 at 25° C. by Lakepharma Inc. Biotinylated B7-1-Fc or CTLA-4-Fc were captured on Streptavidin (SA) biosensors. Loaded biosensors were then dipped into a dilution of either B7-1-Fc or CTLA-4-Fc at variable concentrations (300 nM start, 1:3 down, 7 points). The association rate constant, ka, describes the number of B7-1-CTLA-4 complexes formed per second in a 1 M solution of CTLA-4-Fc or B7-1-Fc.

Impact of Anti-CTLA-4 mAb on Pre-Formed B7-CTLA-4 Complex

For ELISA experiments, hB7-1-Fc or hB7-2-Fc were precoated on 96-well high binding polystyrene plates at given concentrations in coating buffer overnight. After washing away the unbound protein, the plates were blocked with 1% BSA in PBST and then incubated with 0.25 μg/ml biotinylated CTLA-4-Fc protein for two hours. After washing away the unbound protein, given doses of hIgG-Fc/Ipilimumab/L3D10 were added and incubated for 2 hours. The plate-bound biotinylated CTLA-4-Fc was detected with HRP-conjugated streptavidin. For flow cytometric assays, surface hB7-1 or mB7-2 expressing CHO cells (1×10⁵/test) were incubated with soluble biotinylated CTLA-4-Fc (200 ng/test) for 30 min at room temperature. After washing, cells were incubated in 100 μl DPBS buffer for the indicated minutes along with giving doses of control hIgG-Fc or anti-CTLA-4 mAbs. The amounts of B7-bound CTLA-4-Fc were detected with PE-streptavidin by flow cytometry, and the mean fluorescence intensity (MFI) of PE was calculated from triplicated samples.

Tumor Growth and Regression Assay

Mice with either heterozygous or homozygous knock-in of the human CTLA4 gene were challenged with given numbers of either colorectal cancer cell MC38 or melanoma cell line B16-F10. Immunotherapies were initiated at 2, 7 or 11 days after injection of tumor cells with indicated doses. The tumor growth and regression were determined by tumor volume as the readouts. The volumes (V) were calculated using the following formula.

V=ab²/2, where a is the long diameter, while b is the short diameter.

Biostatistics

The specific tests used to analyze each set of experiments are indicated in the figure legends. For each statistical analysis, appropriate tests were selected on the basis of whether the data were normally distributed by using the Shapiro-Wilk test. Data were analyzed using an unpaired two-tailed Student's t test or Mann-Whitney test to compare between two groups, either one-way or two-way ANOVA (analysis of variance) with Sidak's correction for multiple comparisons, two-way repeated-measures ANOVA for behavioral tests. Sample sizes were chosen with adequate statistical power on the basis of the literature and past experience. No samples were excluded from the analysis, and experiments were not randomized except what was specified. Blinding was not done during animal group allocation but was done for some measurements made in the study (i.e., tumor size measuring, flow cytometrical assay of B7 expression). In the graphs, y-axis error bars represent S.E.M. or S.D. as indicated. Statistical calculations were performed using Excel (Microsoft), GraphPad Prism software (GraphPad Software, San Diego, Calif.) or R Software (https://www.r-project.org/).

Results

Ipilimumab does not Block the B7-CTLA-4 Interaction if B7 is Presented on Plasma Membrane

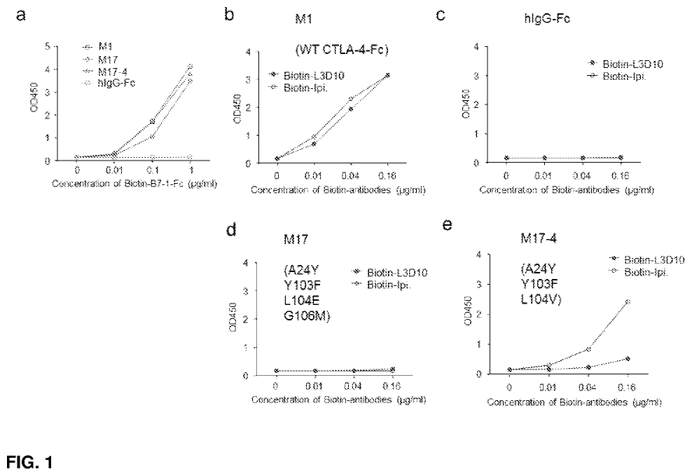

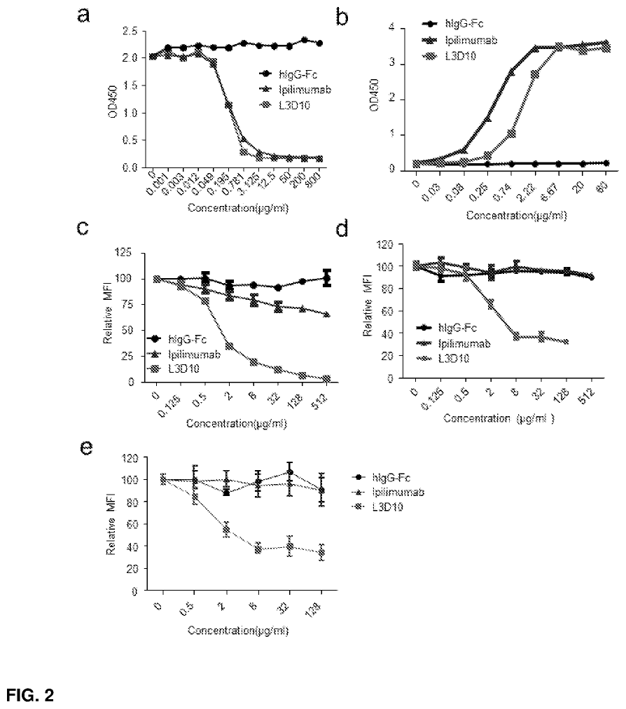

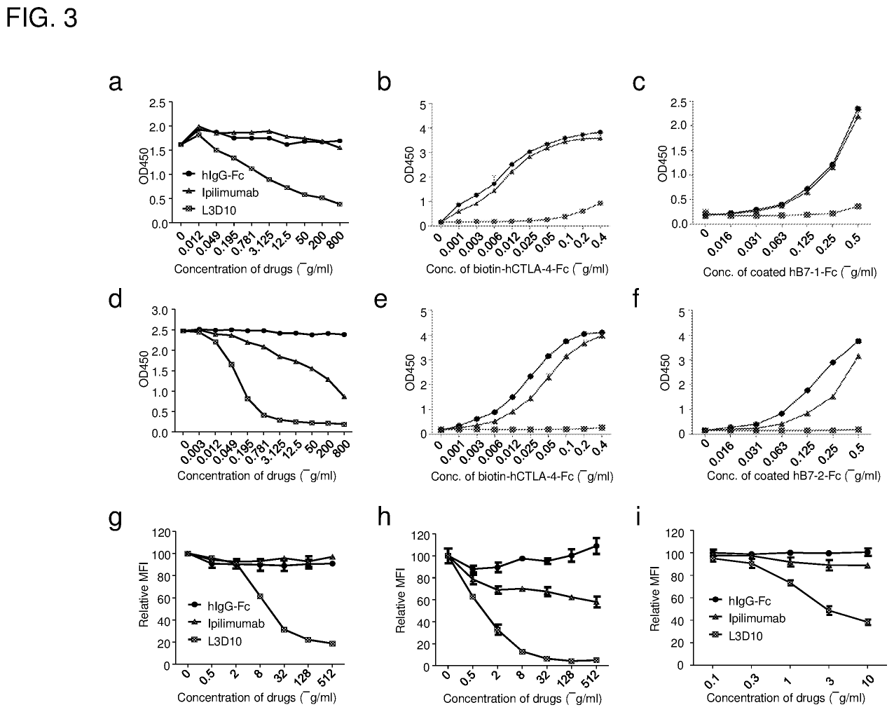

For better comparison, a chimera anti-human CTLA-4 mAb that has the same isotype as Ipilimumab (human IgG1) [14] was produced using the variable region of a mouse anti-human CTLA-4 mAb (L3D10) [15]. The chimera antibody has an apparent affinity of 2.3 nM, which is similar to Ipilimumab (1.8-4 nM) [14, 16]. The two antibodies bind to an overlapping epitope on human CTLA-4 in distinct manner based on their binding to mutant CTLA-4 molecules (FIG. 1). Consistent with the previous report [14], Ipilimumab potently inhibited the B7-1-CTLA-4 interaction when immobilized CTLA-4 is used to interact with soluble B7-1, which is comparable to L3D10 (FIG. 2A). Since B7-1 and B7-2 function as cell surface co-stimulatory molecules, the blockade of anti-CTLA-4 antibodies was evaluated using immobilized human B7-1 and B7-2. As shown in FIG. 3A, Ipilimumab did not block CTLA-4-Fc binding to plate-immobilized hB7-1 even when used at an extremely high concentration (800 μg/ml). The lack of blocking was not due to batch variation of recombinant Ipilimumab, as the same results were obtained using commercial Ipilimumab from three independent sources (including drug used in clinic). In contrast, L3D10 showed significant blocking of plate-immobilized hB7-1 binding at concentrations as low as 0.2 μg/ml, achieving 50% inhibition (IC₅₀) at around 3 μg/ml. Therefore, L3D10 is at least 1,000 fold more efficient than Ipilimumab in blocking B7-1-CTLA-4 interaction when B7-1 is immobilized on the plate. The lack of blocking activity of Ipilimulab was evident across a wide-range of ligand and receptor concentrations (FIGS. 3B and 3C). Binding of plate-immobilized B7-2 to CTLA-4-Fc was somewhat more susceptible to blocking by Ipilimumab, although at a high IC₅₀ of approximately 200 μg/ml (FIG. 3D). Since the IC50 is 10-time higher than the steady plasma levels achieved by the effective dose of 3 mg/kg [7] (19.4 μg/ml, based on company product inserts), it is unlikely that significant blockade of the B7-2-CTLA-4 interaction would be achieved by the clinical doses. The poor blocking activity of Ipilimumab was observed over a wide range of B7-2 and CTLA-4 protein concentrations (FIGS. 3E and 3F). Again, with an IC₅₀ of 0.1 μg/ml, L3D10 is approximately 2,000-fold more efficient than Ipilimumab in blocking B7-2-CTLA-4 interaction. Perhaps the subtle differences between B7-1 and B7-2 can be explained by the fact that the B7-2-CTLA-4 interaction has a higher off rate [17] rather than distinct binding site structures, as structure analyses of the B7-1-CTLA-4 and the B7-2-CTLA-4 complexes show very similar interactions [18-19].

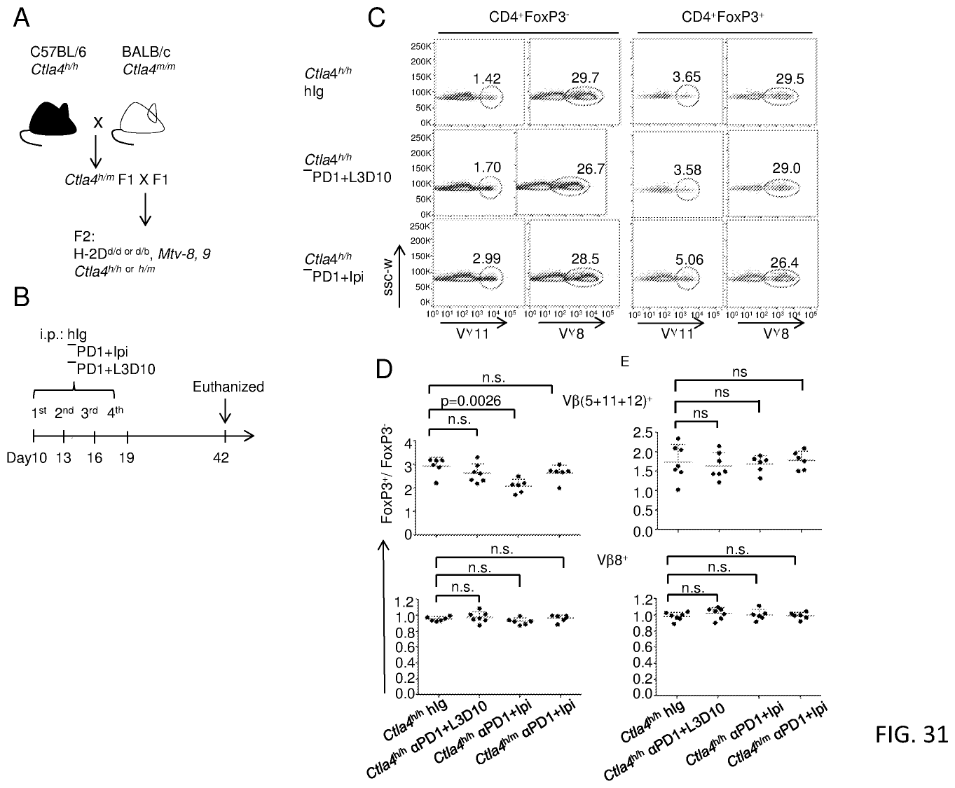

To substantiate this surprising observation, Chinese Ovary Cells (CHO) that express B7 in conjunction with FcR were used [20]. Biotinylated CTLA-4-Fc was used to evaluate the blocking activity of the two anti-human CTLA-4 mAbs. Again, while L3D10 effectively blocked CTLA-4-Fc binding to B7-1-transfected CHO cells, Ipilimumab failed to block even when used at 512 μg/ml (FIG. 3G). While much less potent than L3D10, high doses of Ipilimumab achieved approximately 25% blocking of the interaction between human CTLA-4 and mouse B7-1 (mB7-1) (FIG. 2C). While some blocking of CTLA-4-Fc binding to B7-2- and FcR transfected CHO cell was achieved by Ipilimumab, less than 50% inhibition was observed even when Ipilimumab was used at 512 μg/ml (FIG. 3H). A potential caveat is that biotinylation may have affected binding of Ipilimumab to CTLA-4-Fc. To address this concern, binding of L3D10 and Ipilimumab to biotinylated CTLA-4-Fc used in the blocking studies was compared. As shown in FIG. 2B, Ipilimumab is more effective than L3D10 in binding the biotinylated CTLA-4-Fc. Therefore, the failure in blockade by Ipilimumab was not due to insufficient binding to biotinylated CTLA-4-Fc. A similar pattern was observed when polyhistidine-tagged CTLA-4 was used to interact with B7-1-transfected CHO cells (FIG. 2D). To exclude the possible role of FcR on cell surface, hB7-1 expressing and FcR-negative L929 cells were used. As shown in FIG. 2E, L3D10 but not Ipilimumab blocked CTLA-4 binding to cell surface B7-1. Furthermore, the lack of blocking by Ipilimumab was also observed when LPS-matured spleen dendritic cells were used as the source of B7 (FIG. 31). Taken together, the data suggest that the Ipilimumab's ability to block B7-CTLA-4 interaction is highly dependent on the assay format employed, with minimal to no detectable blocking activity if B7-1 and B7-2 are immobilized, whereas L3D10 is a robust blocker for B7-CTLA-4 interaction regardless of whether the B7 protein is immobilized.

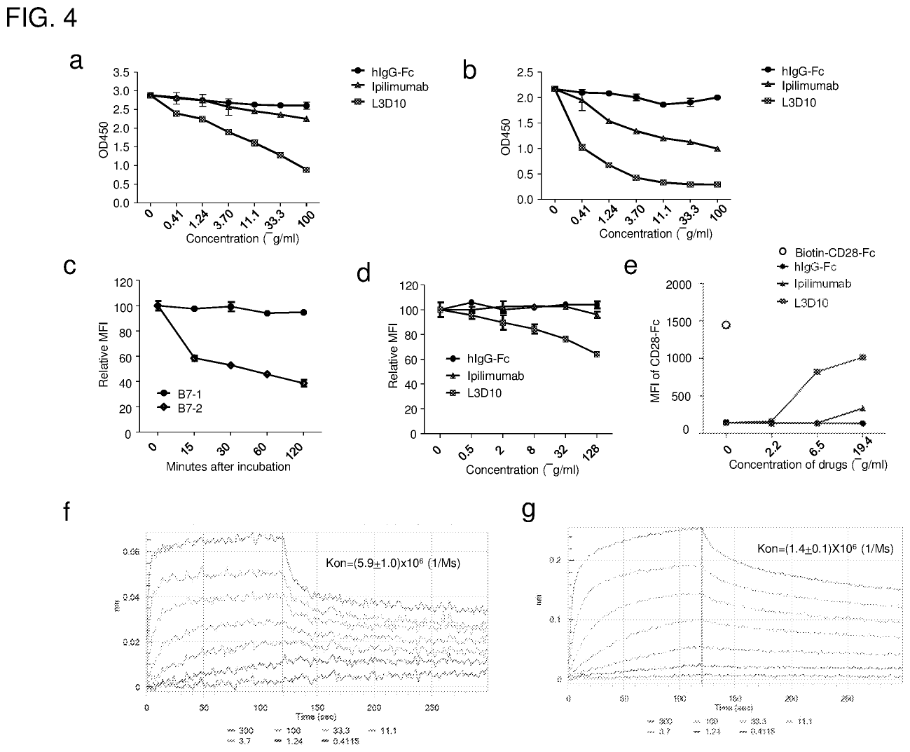

Since CTLA-4 and B7 co-exist in vivo and interact in a dynamic fashion, efficient blocking would require breaking up of pre-existing B7-CTLA-4 complexes. To address this issue, B7 was first allowed to form a complex with biotinylated CTLA-4-Fc. After washing away unbound CTLA-4, grading doses of Ipilimumab or L3D10 is added. After two more hours of incubation, the antibodies and unbound proteins were washed away, and the remaining bound CTLA-4 molecules were detected by HRP-conjugated streptavidin. As shown in FIG. 4A, while L3D10 potently disrupted the pre-existing B7-1-CTLA-4 complex, Ipilimumab failed to do so. Likewise, while high doses of Ipilimumab partially broke B7-2-CTLA-4 complex, it was 250-fold less effective than L3D10 (FIG. 4B).

As the first step to evaluate the impact of anti-CTLA-4 antibodies on pre-formed B7-CTLA-4 complex on cell surface, the stability of the complex was evaluated via flow cytometry by incubating the B7-expressing CHO cells with biotinylated CTLA-4-Fc protein at 4° C. for 0-120 minutes. After washing away disassociated CTLA-4-Fc, PE-conjugated Streptavidin was used to measure cell bound CTLA-4-Fc. As shown in FIG. 4C, the amounts of CTLA-4-Fc on B7-1-expressing CHO cells remained unchanged throughout the 120 minutes of study duration, thus allowing us to test the impact of anti-CTLA-4 antibodies on disrupting the pre-formed B7-1-CTLA-4 complex. In contrast, B7-2-CTLA-4-Fc complex rapidly dissociated within 15 minutes, with majority of the complex collapsed within 30 minutes (FIG. 4C). The rapid disassociation made it impossible to evaluate the impact of anti-CTLA-4 mAbs on pre-formed B7-2-CTLA-4 complex in these assays. As shown in FIG. 4D, Ipilimumab had minimal effect on disrupting the pre-formed B7-1-CTLA-4 complex on cell surface.

Since CTLA-4 has a higher affinity for B7 than CD28-Fc [17, 21], blocking CTLA-4 may relieve its inhibition of CD28-B7 interaction. To test if L3D10 and Ipilimumab can reverse this inhibition, grading amounts of each antibody or control IgG-Fc were added along with biotinylated CD28-Fc and unlabeled CTLA-4-Fc, and the binding of CD28-Fc to B7-1 transfected J558 cells was measured [22]. As shown in FIG. 4E, L3D10 but not Ipilimumab significantly rescued B7-CD28 interaction. The inability of Ipilimumab to break the preformed complex suggests that the kinetics of the B7-CTLA-4 interaction will be a key determinant for the blocking activity of Ipilimumab. Thus, the kinetics of the B7-CTLA-4 interaction were evaluated by using either immobilized B7-1 or CTLA-4. As shown in FIG. 4F, when B7-1 is immobilized, the apparent affinity of bivalent B7-1-Fc and CTLA-4-Fc is 9.9×10⁻¹° M, which is somewhat higher than that when CTLA-4-Fc is immobilized (1.5×10⁻⁹ M) (FIG. 4G). Remarkably, the on rate of CTLA-4 to immobilized B7-1, Kon=5.9×10⁶ (1/Ms) (FIG. 4F), is 4 times higher than that of B7-1 to immobilized CTLA-4, which is 1.4×10⁶ (1/Ms) (FIG. 4G) (P=0.0015). The slower formation of the B7-CTLA-4 complex when B7 is present in solution may allow Ipilimumab to occupy CTLA-4 prior to formation of the B7-CTLA-4 complex which is resistant to breakup by Ipilimumab, thus providing a mechanism to reconcile assay-dependent blocking activity of Ipilimumab. On the other hand, L3D10 can break preformed complex, and can thus block the CTLA-4-B7 interaction regardless of the conditions employed herein.

Ipilimumab does not Effectively Block B7-CTLA-4-Mediated Cell-Cell Interaction and Transendocytosis of B7-1 and B7-2 by CTLA-4

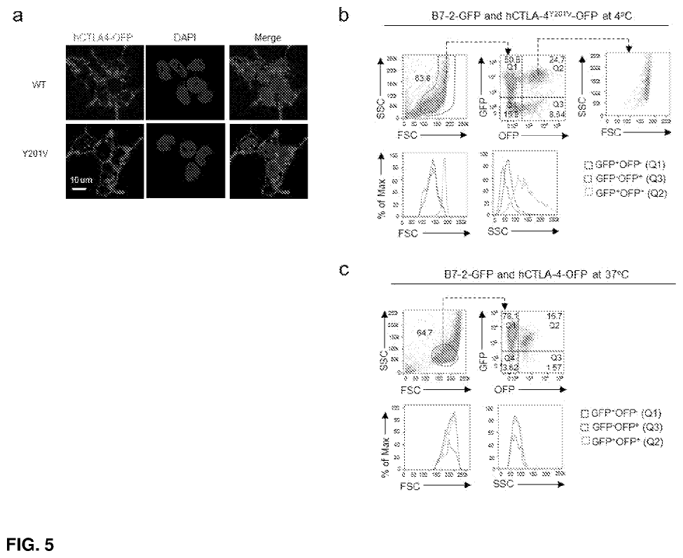

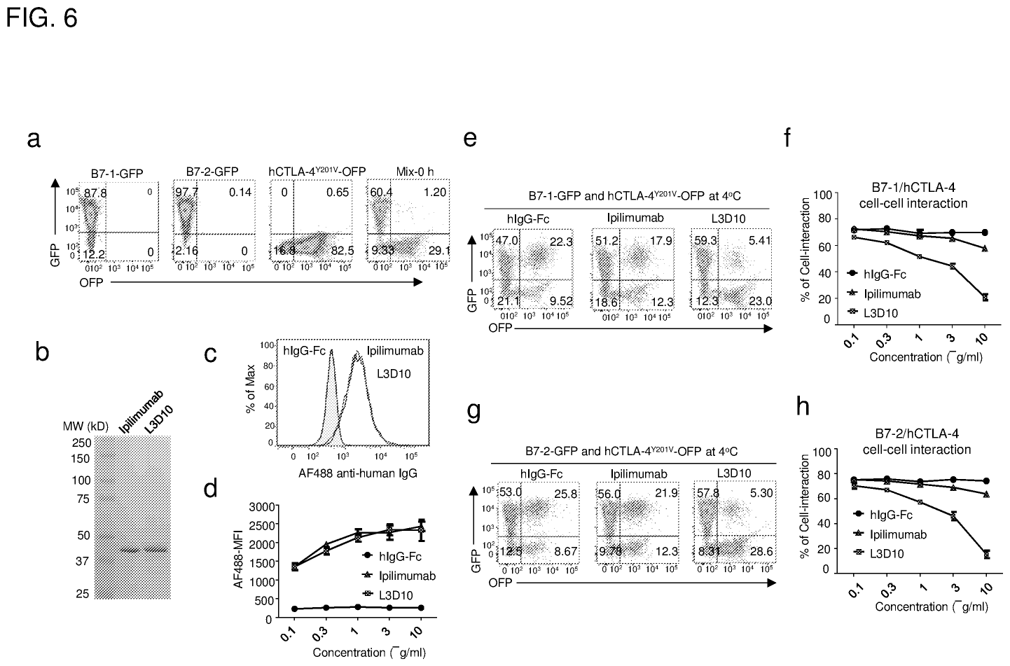

Most CTLA-4 molecules reside inside the cells through AP-2-mediated mechanism [23-24]. In order to measure whether anti-CTLA-4 mAb could block B7-CTLA-4 interaction when they are both stably expressed on cell surface, the Y201V mutation was introduced into CTLA-4 to abrogate its spontaneous endocytosis and thus allow stable cell surface expression [25] (FIG. 5A). As shown in FIG. 6A, the CHO cells expressing either B7-1-GFP or B7-2-GFP and HEK293T cells expressing CTLA-4^{Y201V}-OFP are clearly distinguishable by flow cytometry. When they were mixed immediately prior to FACS analyses, barely any GFP⁺OFP⁺ cells were observed. To compare Ipilimumab and L3D10 for their ability to block cell-cell interaction, Fab fragments were prepared from both antibodies (FIG. 6B) in order to avoid indirect effect caused by cross-linking of CTLA-4 molecules. The antibody Fabs showed comparable binding to cells stably transfected with OFP-tagged CTLA-4 (FIGS. 6C and 3D). After 2 hours of co-incubation at 4° C. without blocking antibody, most of the OFP⁺ cells acquired GFP at equal intensity of the B7-GFP-expressing cells (FIGS. 6E and 3G). Notably, the GFP⁺OFP⁺ cells had forward and side scatters consistent with cell clusters (FIG. 5B). As shown in FIGS. 6E and 6F, effective blocking of B7-1-GFP-CTLA-4^{Y201V} interaction was achieved by L3D10 but not Ipilimumab Fab. Likewise, while only 15% inhibition of cellular B7-2 and CTLA-4 interaction was achieved by 10 μg/ml of Ipilimumab Fab, the same dose of L3D10 Fab caused 80% inhibition (FIGS. 6G and 6H).

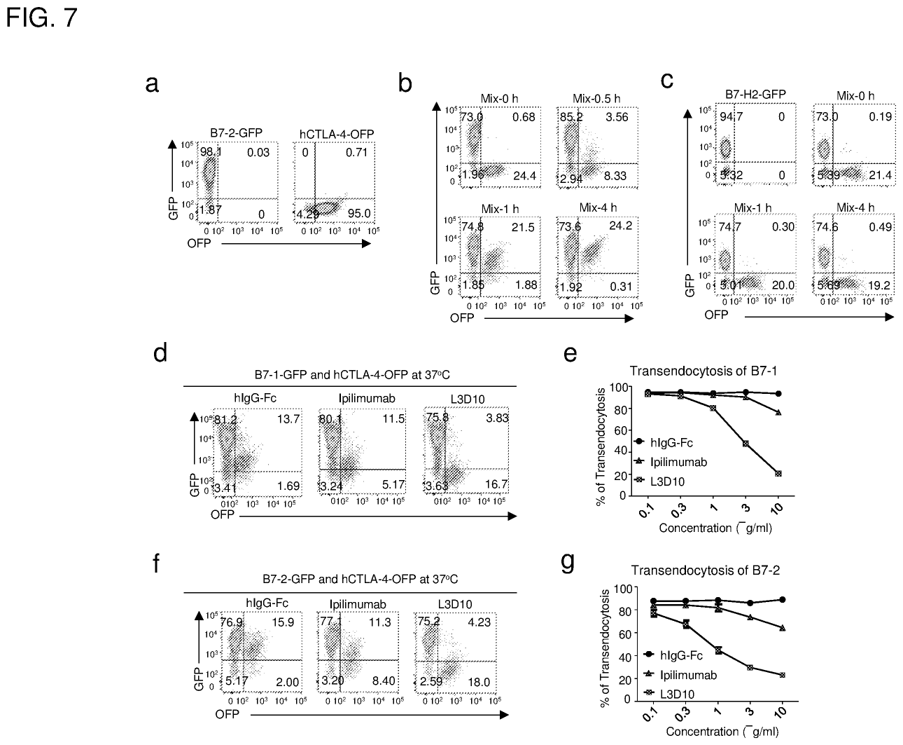

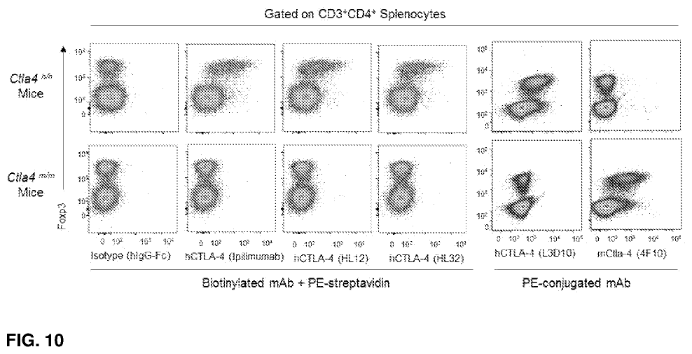

It has been demonstrated that CTLA-4 mediates transendocytosis of cell surface B7-2 [12]. These findings provide us with another assay to measure the blocking activity of anti-CTLA-4 mAbs under more physiologically relevant conditions. CHO cells transfected with either GFP-tagged B7 or OFP-tagged CTLA-4 were used (FIG. 7A). The use of fluorescent protein tagged receptor and ligand allowed us to quantify their interaction in live cells. To ensure CTLA-4-OFP+ cells are surrounded by B7-2-GFP+ cells, excess amounts of B7-2-GFP+ cells were added. As shown in FIG. 7B, co-incubation at 37° C. resulted in a time-dependent accumulation of a new population of cells that expressed both CTLA-4 and B7-2. This accumulation peaked at 4 hours after co-incubation. Since essentially all OFP+ cells had become OFP+GFP+ overtime while the percentage of GFP+ OFP− cells remained unchanged throughout the co-incubation, the appearance of double positive cells was due to uptake of B7-2-GFP by CTLA-4-OFP-transfected CHO cells, as expected. Consistent with this interpretation, the scatters of the OFP+GFP+ are those of single cells (FIG. 10C). As control, the CTLA-4-OFP transfectants were co-cultured with the B7-H2-GFP transfectants. As shown in FIG. 7C, no detectable transfer of GFP signal to the CTLA-4-OFP transfectant was observed over a 4 hour period, thus confirming the specificity of the assay. Having established the model, the impact on transendocytosis by L3D10 and Ipilimunab Fabs was compared. As shown in FIGS. 7D and 4E, the L3D10 Fab is approximately 10-fold more efficient than Ipilimumab Fab in blocking tranendocytosis of B7-1. Similarly, L3D10 Fab is approximately 30-fold more effective in blocking B7-2 transendocytosis (FIGS. 7F and 7G). It should be noted that, while L3D10 Fab effectively blocked transendocytosis of B7-2 (IC50=1 μg/ml), Ipilimumab Fab achieved less than 20% inhibition of B7-1 transendocytosis (FIG. 7E) and only 30% inhibition of B7-2 transendocytosis (FIG. 7G) when used at 10 μg/ml. By molar ratio, this dose is equal to 30 μg/ml of intact Ipilimumab, which is approximately 50% higher than the steady-state plasma drug concentration when an effective dose of Ipilimumab (3 mg/kg) is used in clinic [7].

Ipilimumab does not Block Down-Regulation of B7-1/B7-2 by CTLA-4 In Vivo

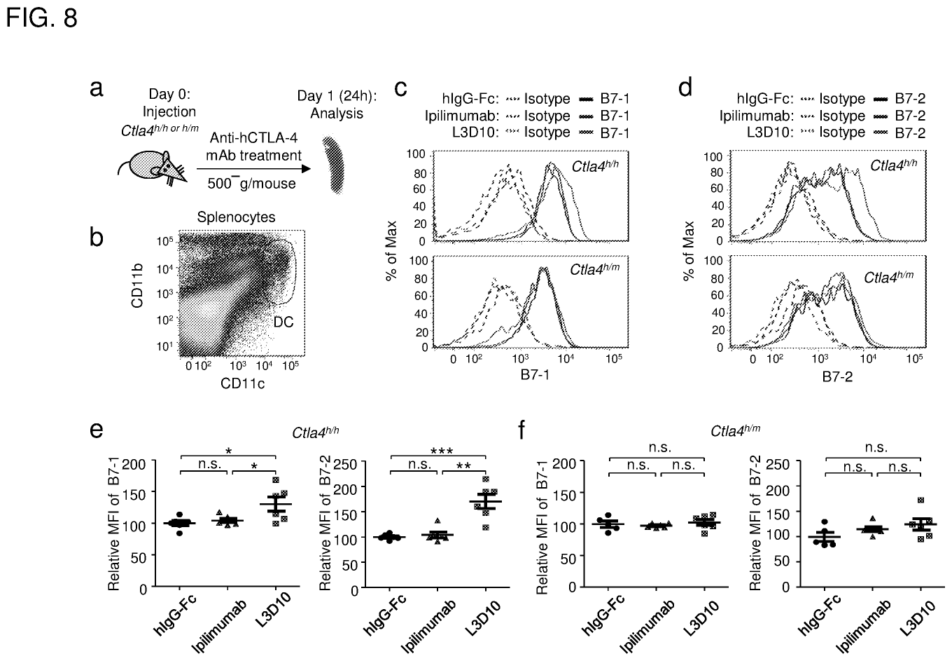

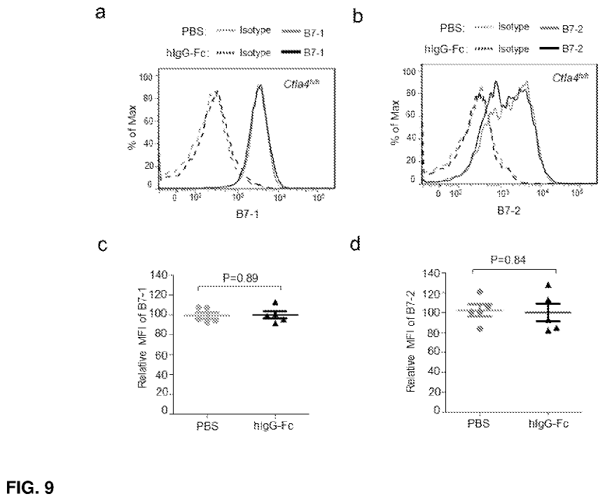

CTLA-4 is expressed predominantly in Treg where it suppresses autoimmune diseases by down-regulating B7-1 and B7-2 expression on dendritic cells (DC) [26] among other potential mechanisms. Since targeted mutation of Ctla4 [26] and treatment with blocking anti-CTLA-4 mAb[12] both increase expression of B7-1 and B7-2 on DC, it has been suggested that the physiological function of CTLA-4 on Treg is to down-regulate B7 on DC through transendocytosis [12, 27]. Therefore, a direct consequence of blocking B7-CTLA-4 interaction is up-regulation of B7 on DC. To evaluate blocking activities of anti-CTLA-4 mAbs in vivo, very high doses of anti-CTLA-4 mAb (500 μg/mouse, which is roughly 25 mg/kg or >8 times the highest Ipilimumab dose used in clinics, 3 mg/kg) were injected into Ctla4^{h/h} or Ctla4^{h/m} mice and harvested spleen cells to measure levels of B7-1 and B7-2 on CD11c^{high} DC at 24 hours after injection (FIGS. 8A and 5B). As shown in FIGS. 8C, 8D and 8E, in comparison to Ctla4^{h/h} mice that received human IgG1-Fc, DC from L3D10-treated mice showed modest but statistically significant elevation of B7-1 and a robust up-regulation of B7-2. The magnitude of up-regulation in B7-2 is comparable to what was achieved using a blocking anti-CTLA-4 mAb in human Treg-DC co-culture [12, 27]. On the other hand, Ipilimumab failed to up-regulate B7-1 and B7-2 in vivo. To rule out the potential effect of contaminating LPS, the endotoxin levels in the antibody preparations was measured, which showed that they were between 0.00025-0.0025 ng/μg, and were 2-10-fold lower than the IgG Fc controls, which did not cause B7-1 and B7-2 up-regulation in vivo (FIG. 9).

Since at least 50% of the CTLA-4 proteins in the Ctla4^{h/m} mice are of mouse origin and do not bind to the anti-human CTLA-4 antibodies (FIG. 10) but functionally cross-react with mouse B7-1 and B7-2 [28-30], and since transendocytosis should only require some unblocked CTLA-4 molecules on Treg, the unbound CTLA-4 should down-regulate B7 on DC even in the presence of blocking anti-human CTLA-4 mAbs. Indeed, neither antibody caused upregulation of B7-1 and B7-2 on DC from Ctla4^{h/m} mice (FIGS. 8C, 8D and 8F). The upregulation of B7 by L3D10 specifically in the Ctla4^{h/h} but not in the Ctla4^{h/m} mice further validates the notion that B7 up-regulation depends on complete blocking of B7-CTLA-4 interaction and ruled out the possibilities that up-regulation of B7 by L3D10 is due to contaminating LPS.

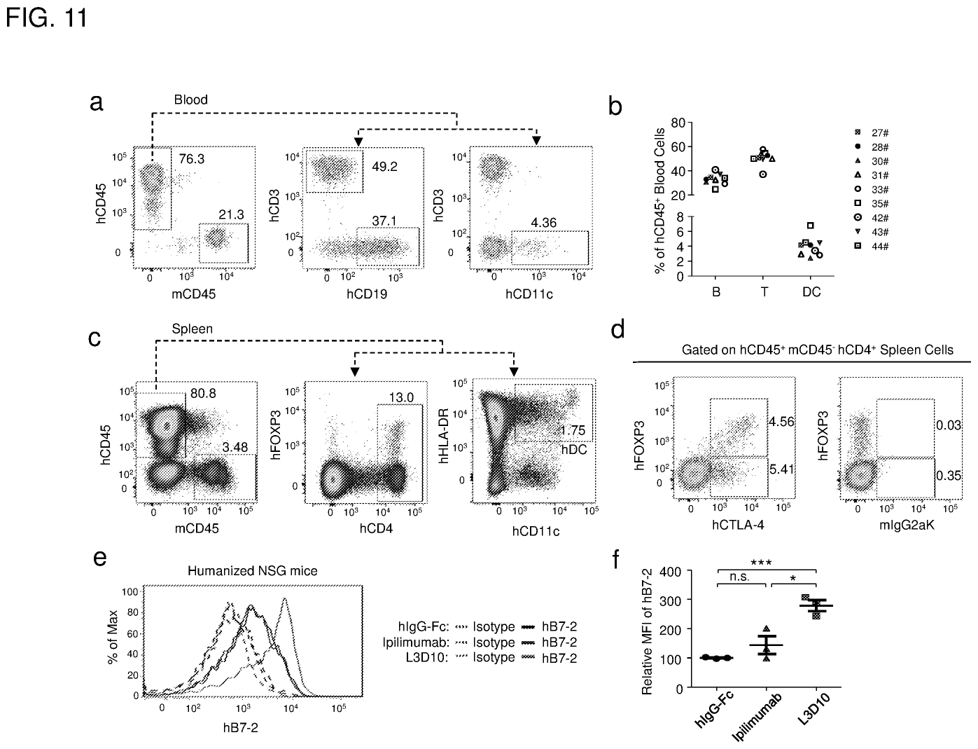

To determine if the lack of blocking by Ipilimumab observed in the Ctla4^{h/h} mice can be observed between human T cells and human dendritic cells, human cord blood CD34⁺ stem cell reconstituted NSG™ mice were employed. As shown in FIGS. 11A and 11B, the peripheral blood of the mice used here consisted of 70-90% of human leukocytes, including T and B lymphocytes and DC. In the spleen, high frequencies of FOXP3⁺ Treg and CD11c⁺ HLA-DR⁺ DC were observed (FIG. 11C). Significant expression of hB7-2 (FIG. 11E) but not hB7-1 (data not shown) was observed on DC. Since human CTLA-4 was expressed at high levels in FOXP3⁺ Treg (FIG. 11D), this model was used to study the B7-2-CTLA-4-interaction between human DC and Treg in an in vivo setting. As shown in FIGS. 11E and 11F, Ipilimumab did not significantly up-regulate B7-2 expression on DC (P=0.22), whereas DC from L3D10-treated mice showed nearly 2.5 fold higher levels of B7-2 (P<0.001), consistent with human CTLA4 gene knockin mouse data. Therefore, L3D10 but not Ipilimumab blocks the down-regulation of B7-2 by hCTLA-4 in human hematopoietic system of humanized mice.

Blocking the B7-CTLA-4 Interaction is Required for Neither Treg Depletion Nor Tumor Rejection

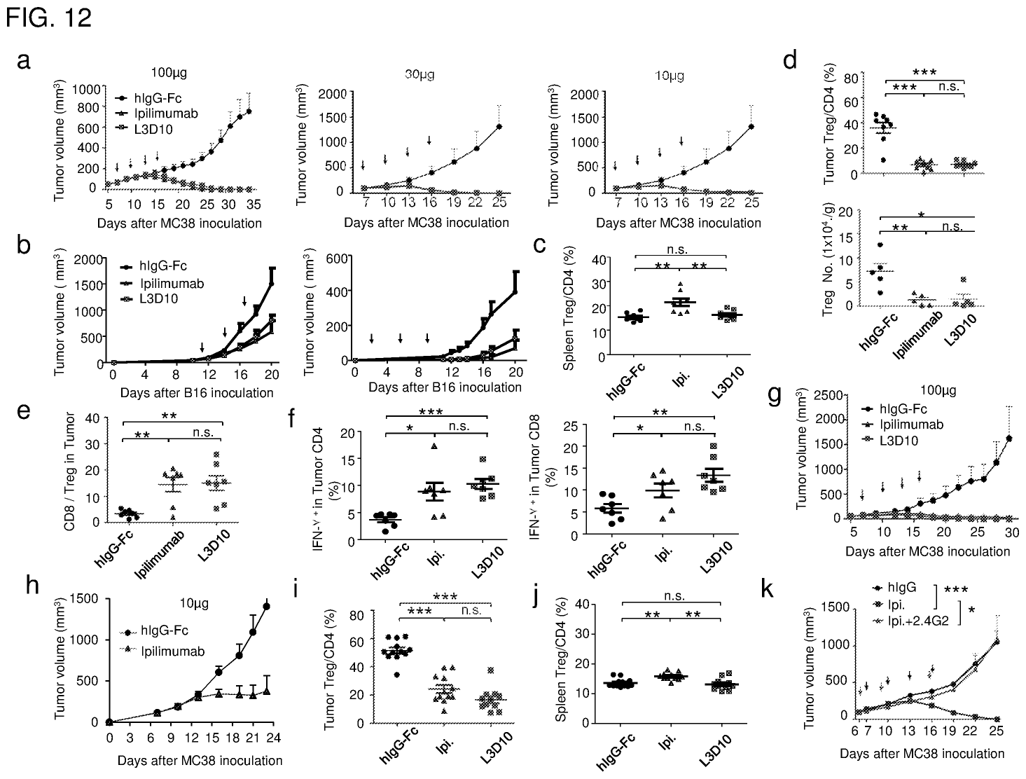

To test whether blockade of the B7-CTLA-4 interaction is required for immunotherapeutic effect, L3D10 and Ipilimumab were first compared for their ability to induce tumor rejection. The Ctla4^{h/h} mice were challenged with colon cancer cell line MC38. When the tumor reached a size of approximately 5 mm in diameter, the mice were treated four times with control human IgG-Fc, L3D10 or Ipilimumab at doses of 10, 30 and 100 μg/mouse/injection and tumor size was observed for 4-6-weeks. As shown in FIG. 12A, while the tumor grew progressively in the control IgG Fc-treated mice, complete rejection was achieved by both anti-CTLA-4 mAbs, even when as low as 10 μg/mouse was used. In multiple experiments, the two antibodies were comparable in causing tumor rejection. In another tumor model, B16 melanoma, both antibodies induced similar retardation of tumor growth, regardless of whether the antibodies were administrated prior to or after tumor was established (FIG. 12B). However, complete rejection was not achieved by antibody monotherapy, as expected from published studies [10].

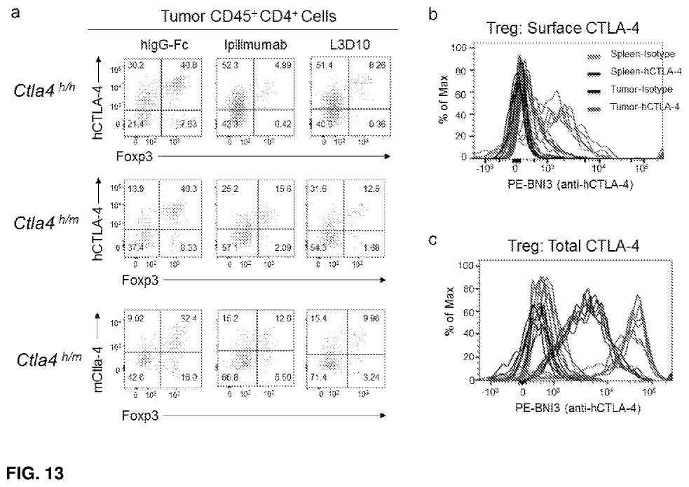

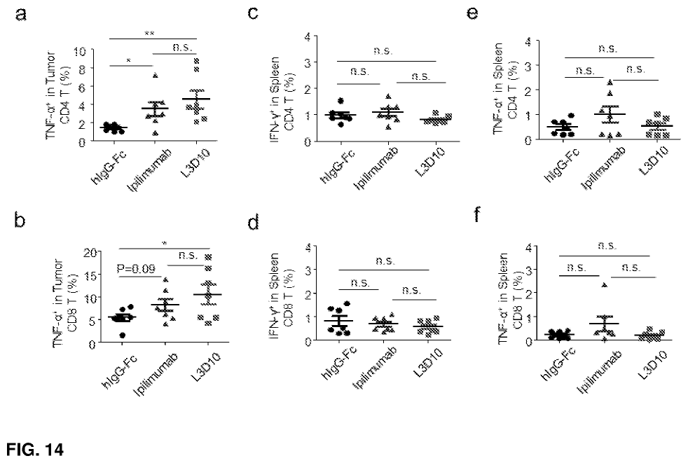

Recent studies have demonstrated that the therapeutic efficacy of anti-mouse Ctla-4 mAbs is affected by the Fc subclass and host Fc receptor, which in turn affect antibody-dependent depletion of Tregs selectively within the tumor microenvironment [9-11]. However, it has not been tested whether such depletion requires blockade of the B7-CTLA-4 interactions. This remains possible as such blockade can up-regulate B7 (FIGS. 8 and 11), which could cause supra-stimulation of CD28, potentially causing T-cell apoptosis [31-32]. To address this issue, tumor-bearing mice were sacrificed before the rejections were completed and analyzed the frequency of Tregs in mice that received control Ig, Ipilimumab or L3D10. While neither anti-CTLA-4 antibody reduced Treg in the spleen (FIG. 12C), both did in the tumor microenvironment, based on the % (FIG. 12D, upper panel) and absolute numbers (FIG. 12D, lower panel) of Tregs. Interestingly, although tumor-infiltrating Foxp3⁻ T cells expressed CTLA-4, albeit at lower levels, they were not depleted by anti-CTLA-4 mAbs (FIG. 13A). The efficient depletion of Tregs in tumor but not spleen or lymph node can be explained by the much higher expression of CTLA-4 on tumor infiltrated Tregs (FIGS. 13B and 13C), which is also reported by previous studies [9-11]. As a result of Treg depletion, the ratio of CD8 T cells over Treg was selectively increased in the tumor (FIG. 12E). Moreover, depletion of Tregs was associated with functional maturation of CD8 and CD4 T cells, as demonstrated by increased interferon γ (IFNγ)-producing cells (FIG. 12F) and tumor necrosis factor α (TNFα)-producing T cells within tumor microenvironment (FIGS. 14A and 14B) but not in the spleen (FIG. 14C-14F).

Since L3D10 and Ipilimumab are comparable in depletion of Tregs in the tumor microenvironment, blockade of the B7-CTLA-4 interaction unlikely contributes to Treg depletion. In addition, since Ipilimumab does not appear to block the B7-CTLA-4 interaction in vivo and still confers therapeutic effect in the Ctla4^{h/h} mice and in melanoma patients, blockade of this interaction is unlikely required for its therapeutic effect. Furthermore, since two mAbs with drastically different blocking activities have comparable therapeutic effects and show similar efficacy in selective Treg depletion in tumor microenvironment, blocking the B7-CTLA-4 interaction does not enhance the therapeutic effect of an antibody. To substantiate this observation, the therapeutic response of the two anti-CTLA-4 mAbs was tested in the Ctla4^{h/m} mice in which the anti-human CTLA-4 mAbs can bind to a maximum of 50% of CTLA-4 molecules and in which neither antibody can block B7-CTLA-4 interaction to achieve upregulation of B7 on dendritic cells (FIG. 8F). Again, both antibodies caused rapid rejection of the MC38 tumors when high doses (FIG. 12G) or lower doses (FIG. 12H) of antibodies were used. Correspondingly, both antibodies selectively depleted Treg in tumor microenvironment (FIG. 12I) but not in the spleen (FIG. 12J). These genetic data further question the relevance of CTLA-4 blockade in both tumor rejection and local Treg depletion and thus dispute the prevailing hypothesis that anti-CTLA-4 mAb induces cancer immunity through blocking the B7-CTLA-4 interaction [4]. Since the therapeutic antibodies were all efficient in Treg depletion but varied in their ability to block B7-CTLA-4 interaction, it was hypothesized that these antibodies caused tumor-rejection by inducing Treg depletion through antibody-dependent cellular cytotoxicity (ADCC). Since ADCC is dependent on FcR on the host effector cells, it was tested if anti-FcR antibodies can abrogate tumor rejection. As shown in FIG. 12K, concurrent anti-FcR treatment completely erased tumor immunotherapeutic effect of Ipilimumab.

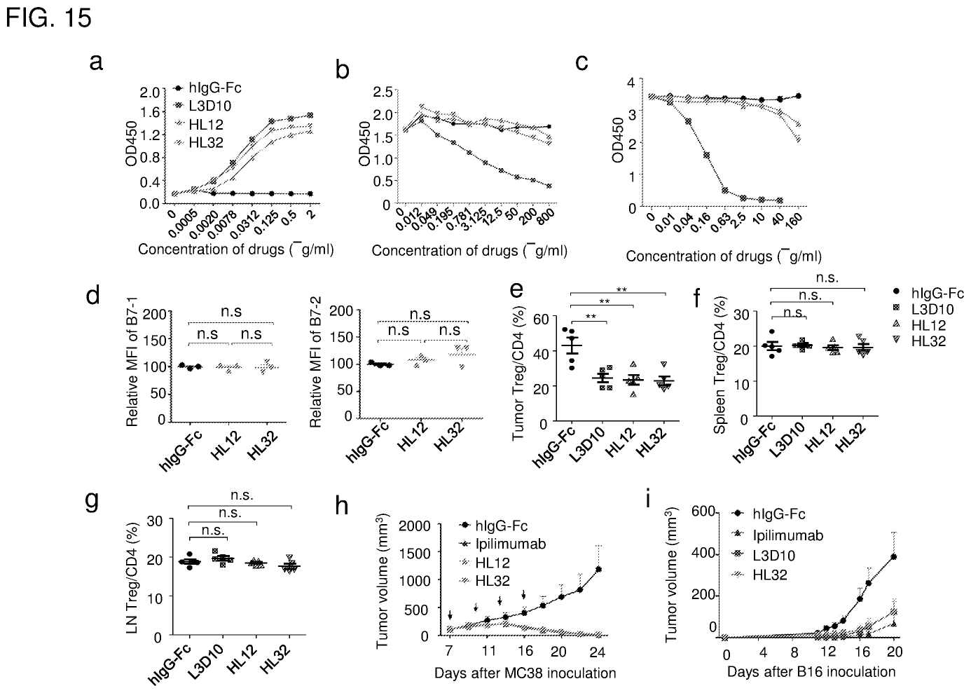

During humanization of L3D10 mAb, two clones called HL12 and HL32 were obtained, which retained potent binding to CTLA-4 (FIG. 15A) but lost the ability to block CTLA-4 binding to plate bound B7-1 (FIG. 15B) and B7-2 (FIG. 15C), perhaps due to an approximately 4-fold increase in off-rate and correspondingly bivalent avidity (Table 1).

Multi-concentration kinetic experiments were performed on the Octet Red96 system (ForteBio). Anti-hIgG-Fc biosensors (ForteBio, #18-5064) were hydrated in sample diluent (0.1% BSA in PBS and 0.02% Tween 20) and preconditioned in pH 1.7 Glycine. The antigen was diluted using a 7-point, 2-fold serial dilution starting at 600 nM with sample diluent. All antibodies were diluted to 10 μg/ml with sample diluent and then immobilized onto anti-hIgG-Fc biosensors for 120 seconds. After baselines were established for 60 seconds in sample diluent, the biosensors were moved to wells containing the antigen at a series of concentrations to measure the association. Association was observed for 120 seconds and dissociation was observed for 180 seconds for each protein of interest in the sample diluent. Kₒₙ, on rate; K_{dis}, off rate; KD, the equilibrium dissociation constant.

TABLE 1
Binding characteristics of humanized
L3D10 clones used in this study.

| Antibodies | Antigen | KD (M) | Kₒₙ(1/Ms) | K_{dis}(1/s) |
|---|---|---|---|---|
| HL12 | CTLA4Fc | 7.2E−09 | 2.3E+05 | 1.6E−03 |
| HL32 | CTLA4Fc | 7.1E−09 | 2.7E+05 | 1.9E−03 |
| L3D10 | CTLA4Fc | 2.3E−09 | 3.5E+05 | 8.0E−04 |

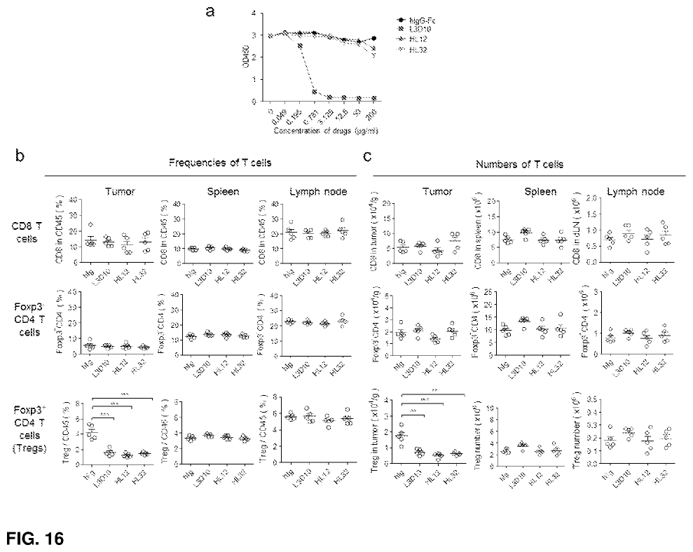

Correspondingly, these antibodies also lost the ability to induce up-regulation of B7-1 and B7-2 on host APC (FIG. 15D). The ability of the antibodies to block soluble B7 binding to immobilized CTLA-4-Fc was also abrogated (FIG. 16A). The fact that these humanized antibodies have lost the ability to block B7-CTLA-4 interaction provides us with an opportunity to further test whether the blocking activity is essential for tumor rejection and Treg depletion. As shown in FIG. 15E, despite the loss of blocking activity, the humanized antibodies rapidly induced Treg depletion in tumor microenvironment but not in spleen (FIG. 15F) or draining lymph node (FIG. 15G). Furthermore, HL12 and HL32 exhibited similar effects as L3D10 on abundance of T cell subpopulations in peripheral lymph organs and tumors (FIGS. 16B and 16C). More importantly, both antibodies were as effective as Ipilimumab and parental L3D10 in causing rejection of MC38 (FIG. 15H) and B16 (FIG. 15I) tumors.

B7-CTLA-4 Interaction is not Required for the Immunotherapeutic Activity of Ipilimumab

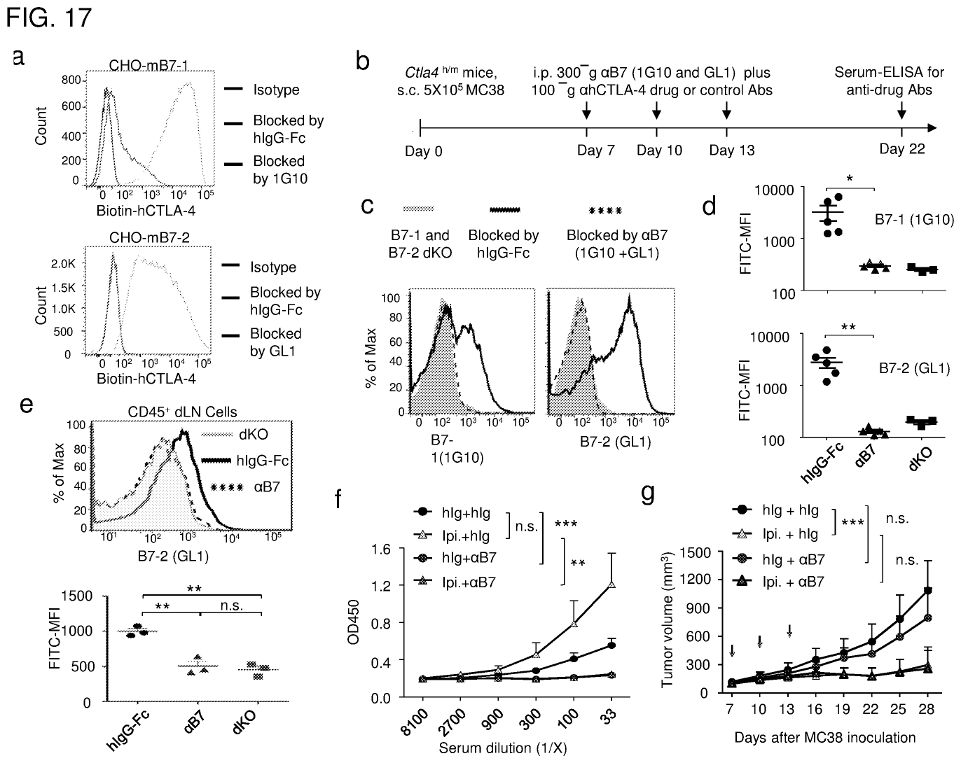

A critical prediction of the CTLA-4 checkpoint blockade hypothesis is that anti-CTLA-4 mAb should not confer immunotherapeutic effect unless B7 is present to deliver a negative signal. Since mice with targeted mutations of Cd80 (encoding B7-1) and Cd86 (encoding B7-2) do not have Treg [33] and thus express very little Ctla4, this prediction was tested by using a saturating dose of anti-B7-1 (1G10) and anti-B7-2 (GL1) mAbs, which block binding of human CTLA-4 to mB7-1 and mB7-2, respectively (FIG. 17A). As diagrammed in FIG. 17B, MC38 tumor-bearing mice were treated with Ipilimumab in conjunction with either control Ig or a combination of anti-mB7-1 and anti-mB7-2 mAbs. The anti-mB7 mAbs used completely masked all B7-1 and B7-2 in the peripheral blood leukocytes as their binding to new anti-mB7 mAb was reduced to what was observed in mice with targeted mutations of both Cd80 and Cd86 (dKO) (FIGS. 17C and 17D). Similar blocking of B7-2 was observed for DC from tumor draining lymph node (FIG. 17E). However, the levels of B7-1 were barely detectable by 1G10 mAb regardless of antibody treatment (data not shown), making it impossible to evaluate the extent of masking of endogenous B7-1. Since B7-1 and B7-2 are both required for antibody response to antigens [34], and since anti-CTLA-4 antibodies are potent inducer of anti-drug antibodies (ADA) [5], ADA is a good indicator for function of both B7-1 and B7-2 in vivo. Functional blocking was further confirmed by the fact that antibody response to Ipilimumab was completely abrogated (FIG. 17F). Importantly, saturating blockade of B7 did not affect the Ipilimumab-induced tumor rejection as anti-mB7 and control Ig treated mice were equally responsive to Ipilimumab therapy (FIG. 17G). Therefore, abrogation of negative signaling by B7 does not explain immunotherapeutic effect of Ipilimumab.

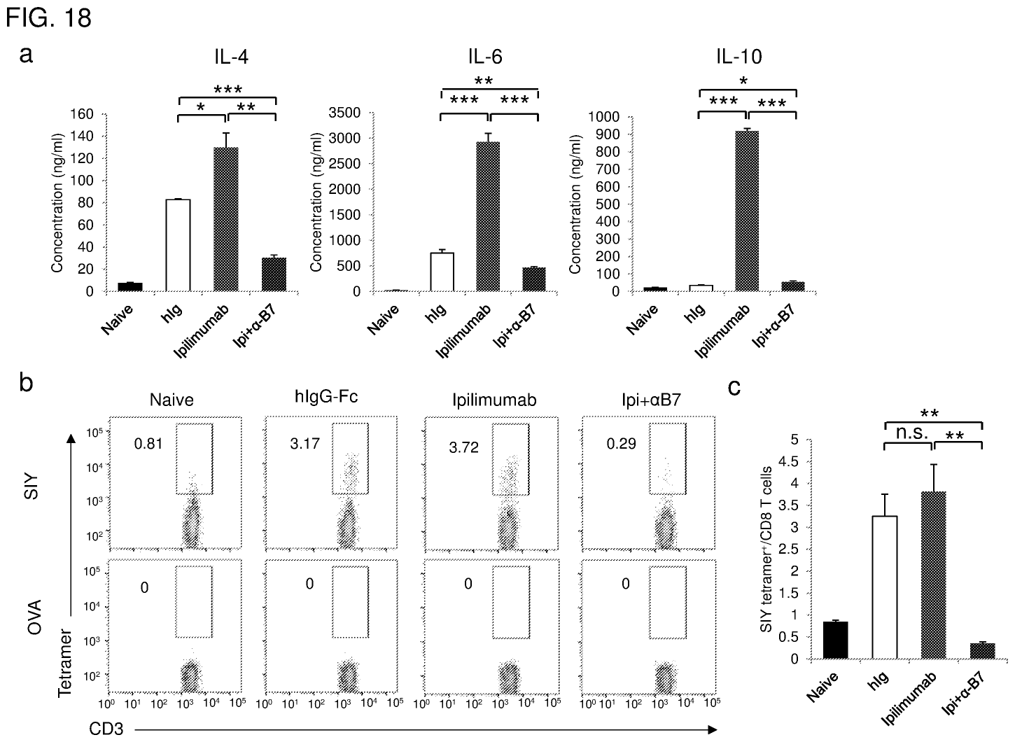

Another key prediction of the checkpoint blockade hypothesis is that anti-CTLA-4 mAb releases breaks of naïve T cells to achieve cancer immunotherapeutic effect. Since anti-B7 mAbs completely abrogated T-cell-dependent antibody responses, it was tested if the in vivo treatment of anti-B7 mAbs prevented Ipilimumab induced Th2 cell activation. As shown in FIG. 18A, Ipilimumab treatment significantly enhanced the in vitro production of Th2-type cytokines, including IL-4, IL-6 and IL-10. This was abrogated by anti-B7 mAbs treatment in vivo. To test the impact of anti-B7 mAbs on de novo priming of CD8 T cells in the periphery lymphoid organ, tumor-bearing mice were immunized with SIY peptide and treated the mice with Ipilimumab in the presence or absence of anti-B7 mAbs. Representative profiles or SIY-H-2K^{b}-specific T cells are shown in FIG. 18B, while summary data from a representative study are presented in FIG. 18C. As shown in FIGS. 18B and 18C, in the absence of anti-B7 mAbs, immunization with SIY peptide induced significant expansion of SIY-specific T cells. This appears to be slightly increased by Ipilimumab. In the presence of anti-B7 mAbs, however, no de novo priming of SIY-specific T cells was observed. Since the data in FIG. 17 showed that anti-B7 mAbs did not interfere with immunotherapeutic effect of Ipilimumab, the data in FIG. 18 suggest that de novo T cell priming after Ipilimumab treatment is not required for achieving the immunotherapeutic effects by Ipilimumab.

Blocking the B7-Ctla-4 Interaction is not Associated with Immunotherapeutic Effect of Anti-Mouse Ctla-4 mAbs

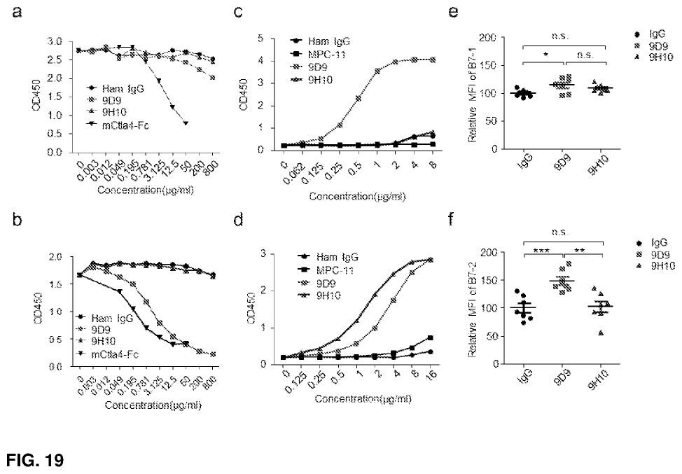

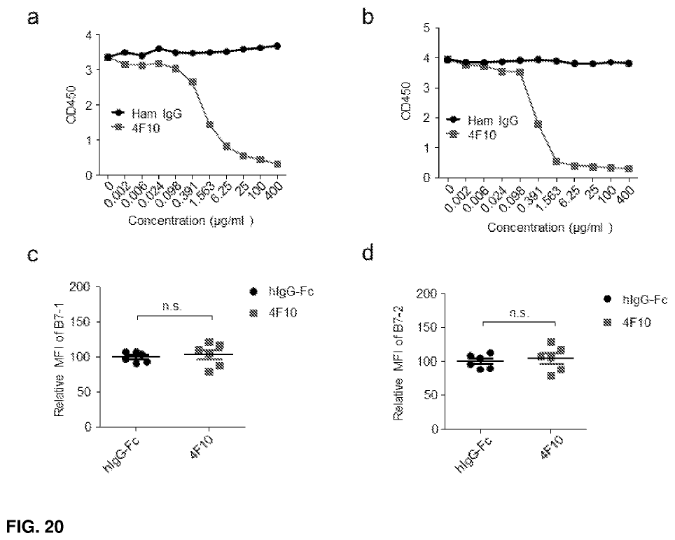

The concept that CTLA-4 is a cell-intrinsic negative regulator for T cell regulation was proposed based on the stimulatory effect of both intact and Fab of two anti-mouse Ctla-4 mAbs[35-36], 4F10 and 9H10, although no data were presented to demonstrate that these antibodies block the B7-Ctla-4 interaction. More recently, a third anti-mouse Ctla-4 mAb, 9D9, was reported to have therapeutic effect in tumor bearing mice and cause local depletion of Treg in tumor microenvironment [10]. Thus, all three commercially available anti-mouse Ctla-4 mAbs that had been shown to induce tumor rejection were tested for their ability to block the B7-Ctla-4 interaction under physiologically relevant conditions. As a first test, increasing amounts of anti-mouse Ctla-4 mAbs (up to 2,000 fold molar excess over Ctla-4-Fc) were used to block binding of biotinylated Ctla-4-Fc to plate-coated mB7-1 and mB7-2. As shown in FIG. 19A, anti-mouse Ctla-4 mAb 9H10 did not block the mB7-1-Ctla-4 interaction even at the highest concentration tested, although a modest blocking was observed when 9D9 was used at very high concentrations. Whereas mAb 9D9 effectively blocked the mB7-2-Ctla-4 interaction, 9H10 failed to do so (FIG. 19B). Interestingly, while 9D9 showed strong binding to soluble Ctla-4Fc, 9H10 showed poor binding (FIG. 19C), even though it was more potent than 9D9 in binding immobilized mouse Ctla-4Fc (FIG. 19D). Since lack of any blocking activity by 9H10 in this assay may simply reflect its poor binding to soluble Ctla-4Fc, again up-regulation of B7-1 and B7-2 on dendritic cells in WT mice (Ctla4^{m/m}) was used to measure in vivo blocking of the B7-Ctla-4 interaction. As shown in FIGS. 19E and 19F, 9H10 did not upregulate B7-1 expression on DCs, while 9D9 increased mB7-1 level by 15% (P<0.05). Interestingly, while 9D9 clearly upregulated mB7-2 on DC, 9H10 failed to do so. Therefore, 9H10, the first and most extensively studied tumor immunotherapeutic anti-Ctla-4 mAb does not block the B7-Ctla-4 interactions. These data argue against a role for blocking the B7-Ctla-4 interaction in the induction of anti-tumor immunity by anti-mouse Ctla-4 mAbs. Since both mAbs showed comparable immunotherapeutic effect and comparable depletion of Treg in the tumor microenvironment [10], local deletion of Treg, rather than blockade of mB7-CTLA-4 interaction, provides a unifying explanation for therapeutic effect of anti-mouse CTLA-4 mAbs. Interestingly, while 4F10 blocked the B7-Ctla-4 interaction in vitro, it failed to induce upregulation of B7 on DCs in vivo (FIG. 20).

Discussion

Although Ipilimumab was called a blocking mAb based on the fact that it blocks the B7-CTLA-4 interaction when B7 is added in soluble form, the data demonstrated that it barely blocks B7-CTLA-4 interaction under physiologically relevant conditions, including those when B7-1 and B7-2 were immobilized to solid phase or expressed on cell membrane, when the B7-CTLA-4 complex was formed prior to exposure to anti-CTLA-4 mAbs, when both B7 and CTLA-4 were expressed as cell surface molecules, and particularly when B7 and CTLA-4 were presented as naturally expressed on DC and T cells respectively and when animals receive antibody treatment in vivo. More importantly, Ipilimumab confers its immunotherapeutic effect without blocking the B7-CTLA-4 interaction because it remains effective either when at least 50% of CTLA-4 does not bind to the antibody in Ctla4^{h/m} mice or when host B7 is masked by anti-B7 mAbs.

A surprising finding in the study described herein is the marked difference in Ipilimumab blocking activity depending on whether B7 or CTLA-4 proteins are placed in soluble phase. This can now be explained by two pieces of data. First, Ipilimumab does not break existing B7-CTLA-4 complexes. Second, the on-rate for soluble CTLA-4 binding to plate-bound B7 is at least three times as fast as that of soluble B7 binding to plate-bound CTLA-4. In combination, these data suggest that when B7 is added in solution, Ipilimumab has more chance than when B7 is immobilized to bind to free CTLA-4 and has more chance to block the CTLA-4-B7 interaction before the complex is formed. Since the CTLA-4-antibody interaction is dynamic, the CTLA-4 molecules that disassociate from antibody could bind to immobilized B7 and becomes “immune” to blocking by Ipilimumab. As such, a partial overlap between B7- and Ipilimumab-binding sites, on CTLA-4, as recently reported [37], does not necessarily enable it to block the B7-CTLA-4 interaction under physiologically relevant conditions.

The differential activity between L3D10 and Ipilimumab to break preformed complex remains to be elucidated. While Kon of Ipilimumab (2.6×10⁵/Ms or 3.83×10⁵/Ms) [14, 16], is lower than that of soluble B7 (1-4×10⁶/Ms, this study), L3D10 does not have a faster Kon (2.07×10⁵/Ms) than Ipilimumab [15]. Therefore, the Kon or Koff does not offer an explanation for the ability of the two antibodies to differentially block B7-CTLA-4 interaction with immobilized B7. A more plausible explanation is that once the complex is formed, the CTLA-4 conformation is changed in such a way as to prevent Ipilimumab from binding it. The published data on Ipilimumab-CTLA-4 complex show partial overlap between Ipilimumab epitope and B7-binding site on CTLA-4 [37], which is consistent with this explanation.

To model the physiological conditions under which both B7 and CTLA-4 are present on cell surface, a transendocytosis assay using CHO cells respectively expressing either GFP-tagged B7-1 or B7-2 or OFP-tagged CTLA-4 was performed. To overcome the complication associated signaling through the cross-linking of CTLA-4, it is important to use Fab rather than bivalent antibodies. The data clearly demonstrate that despite robust binding to cell surface CTLA-4, at concentration that is 10-fold more than needed for saturating binding (10 μg/ml), Ipilimumab Fab caused only 15-30% inhibition of transendocytosis of B7-1 and B7-2. More importantly, by molar ratio, this concentration would translate to approximately 50% higher concentration than steady plasma concentration achieved by clinically effective dosing. Likewise, when cell surface CTLA-4 is stabilized by Y201V mutation to allow stable B7-CTLA-4-mediated cell-cell interaction, the high-doses of Ipilimumab Fab only cause less than 20% inhibition. Since the clinical effective dosing is inadequate to cause effective inhibition of neither B7 transendocytosis nor cell surface interaction mediated by B7 and CTLA-4, the cell-based in vitro assays strongly argue against CTLA-4 blockade as the mechanism of action for the clinically effective drug.

The predictions from these in vitro studies are validated by the in vivo studies. Our in vivo assay is based on the recent discovery that CTLA-4 functions by causing down-regulation of B7 on dendritic cells via transendocytosis [12, 27]. Because of this unique property, one would not expect stable DC-Treg conjugation mediated by B7-CTLA-4 interactions in vivo. Rather, blocking CTLA-4-mediated transendocytosis directly results in higher expression of B7 on DC [12, 27]. To rule out a potential caveat that upregulation of B7 is due to signaling by anti-CTLA-4 mAbs, the heterozygous mice consisting of both mouse and human CTLA4 alleles were used [38]. In this model, anti-human CTLA-4 mAbs can be an effective agonist but not antagonist because it will not be able to bind 50% of CTLA-4 molecules. The fact that blocking anti-CTLA-4 mAb L3D10 induces B7 upregulation in the homozygous but not heterozygous mice confirmed the specificity of the in vivo assay and showed that functional blocking would need block more than 50% of CTLA-4, perhaps because transendocytosis can be accomplished with 50% or less unoccupied CTLA-4. As such, up-regulation of B7 on dendritic cells represents the most physiologically relevant and direct readout for blockade of the B7-CTLA-4 interaction.

The lack of contribution from B7-CTLA-4 blockade is also demonstrated by absence of correlation between blocking and therapeutic efficacy. Despite more than 1000-fold differences in blocking B7-CTLA-4 interaction, L3D10 and Ipilimumab are comparable in inducing tumor rejection. Therefore, such blockade does not significantly contribute to the efficacy of the anti-CTLA-4 mAbs. Interestingly, since L3D10 efficiently induces tumor rejection in heterozygous mice in which it cannot functionally block all the B7-CTLA-4 interaction, such blockade is not necessary for tumor rejection even for a blocking antibody. Remarkably, humanized L3D10 progenies that have lost its blocking activities remain fully active in immunotherapy. These data refute the hypothesis that anti-CTLA-4 mAbs operate primarily through checkpoint blockade [1]. By refuting the prevailing hypothesis, the data suggest that improving the blocking activities of the anti-CTLA-4 mAbs is unlikely the right approach to increase the therapeutic efficacy of anti-CTLA-4 mAb. Our companion paper further validated this concept.

A small proportion of human subject is known to express soluble B7-1 [39]. Since Ipilimumab blocks the interaction between soluble CD80 and CTLA-4, it is of interest to consider whether blocking soluble CD80 may be responsible for tumor rejection. This this unlikely for two reasons. First, since soluble CD80 is known to promote tumor rejection as it provides costimulation for T cells [40], blocking this interaction should suppress rather than promote tumor rejection. Second, the humanized L3D10 clones HL12 and HL32, which lost the ability to block B7-CTLA-4 interaction regardless of whether CD80 is immobilized or in soluble form, are potent inducers of tumor rejection.

Meanwhile, the in vivo studies showed that all therapeutically effective anti-CTLA-4 antibodies used herein are remarkably effective in causing local Treg depletion. Our data provide a piece of clear evidence that, much like anti-mouse Ctla-4 mAbs, anti-human CTLA-4 mAbs, including the clinically effective Ipilimumab, may have provided therapeutic effect through ADCC. This hypothesis is verified by a critical role for host FcR in Ipilimumab-induced tumor rejection. Our work supports the hypothesis that local depletion of Treg within the tumor environment is the main mechanism for clinically effective anti-human CTLA-4 mAb, and hence suggests new approaches to develop the next generation of anti-CTLA-4 mAb for cancer immunotherapy by selectively enhancing local Treg depletion regardless of blocking activity.

The requirement for induction of local Treg depletion within tumor microenvironment to achieve therapeutic effects is inconsistent with another postulate of checkpoint blockade hypothesis [1], which states that unlike anti-PD-1/PD-L1 antibodies, anti-CTLA-4 antibodies promote tumor rejection by preventing negative signaling in the periphery lymphoid organ. By showing that B7 blockade prevented de novo T cell activation without affecting therapeutic effect of Ipilimumab, the data refuted this postulate. Importantly, instead of contributing to tumor rejection, it has been demonstrated that systemic T cell activation strongly correlates to immunotherapy-related adverse effect.

Finally, accumulating genetic data in the mice suggest that the original concept [35-36] that CTLA-4 negatively regulates T cell activation and that such regulation was achieved through Shp-2 [41-42] may need to be revisited [43]. Thus, while the severe autoimmune diseases in Ctla4⁻⁷ mice have been used to support the notion of CTLA-4 as a cell-intrinsic negative regulator for T cell activation [44-45], at least three lines of genetic data have since emerged that are not consistent with this view. First, lineage-specific deletion of the Ctla4 gene in Treg but not in effector T cells is sufficient to recapitulate the autoimmune phenotype observed in mice with germline deletion of the Ctla4 gene [26], although the onset of fatality is slower than mice with either germline or pan-T cell deletion of the gene [44-46]. While the function of Ctla4 in Foxp3⁻ cells remains to be investigated, these data suggest that development of fatal autoimmunity in the Ctla4⁻⁷ mice does not require deletion of Ctla4 in effector T cells. Second, in chimera mice consisting of both WT and Ctla4^{-/-} T cells, the autoimmune phenotype was prevented by the co-existence of WT T cells [47]. These data again strongly argue that autoimmune diseases were not caused by lack of cell-intrinsic negative regulator. The lack of cell-intrinsic negative regulator effect is also demonstrated by the fact that in the chimera mice, no preferential expansion of Ctla4^{-/-} T cells was observed during viral infection [48]. Third, T-cell specific deletion of Shp2, which was proposed to be mediating negative regulation of CTLA-4 [41-42], turned out to reduce rather than enhance T cell activation [49]. In the context of these genetic data reported since the proposal of CTLA-4 as negative regulator for T cell activation, the data reported herein call for a reappraisal of the CTLA-4 checkpoint blockade hypothesis in cancer immunotherapy.

### Example 2

### Complete CTLA-4 Occupation, Systemic T Cell Activation and Preferential Expansion of Self-Reactive T Cells are Dispensable for Tumor Rejection but Correlate with irAE, while Blocking B7-CTLA-4 Interaction Impacts Neither Safety Nor Efficacy of Anti-CTLA-4 Antibodies

Methods

Animals

CTLA4 humanized mice that express the CTLA-4 protein with 100% identity to human CTLA-4 protein under the control of the endogenous mouse Ctla4 locus have been described [24]. The homozygous knock-in mice (Clta4^{h/h}) were backcrossed to the C57BL/6 background for at least 10 generations. Heterozygous mice (Ctla4^{h/m}) were produced by crossing the CTLA4^{h/h} mice with either wild type (WT) BALB/c mice (for tumor growth studies) or WT C57BL/6 mice (for irAE studies). WT BALB/c and C57BL/6 mice were purchased from Charles River Laboratories through an NCI contract. All mice were maintained at the Research Animal Facility of Children's Research Institute at the Children's National Medical Center. All studies involving mice were approved by the Institutional Animal Care and Use Committee.

Cell Culture

Murine colon tumor cell line MC38 was described previously [2], and CT-26 and B16-F10 cell lines were purchased from the ATCC (Manassas, Va., USA). After receiving from vendors, cell passages were kept minimal before in vivo testing. Cell lines were neither authenticated nor regularly tested for mycoplasma contamination. MC38, CT26 and B16-F10 cell lines were incubated at 37° C. with 5% CO₂. MC38 and B16 cells were grown in DMEM (Dulbecco's Modified Eagle Medium, Gibco) supplemented with 10% FBS (Hyclone), 100 units/ml of penicillin and 100 μg/ml of streptomycin (Gibco). CT26 cells were cultured in complete RPMI 1640 Medium (Gibco).

Antibodies

Mouse anti-human CTLA-4 mAb L3D10 has been described [28]. Anti-CTLA-4 mAb L3D10 used in the study was a chimera antibody consisting of human IgG1 Fc and the variable regions of L3D10. Recombinant antibody was produced by Lakepharma, Inc (Belmont, Calif., USA) through a service contract. Recombinant Ipilimumab with the amino acid sequence disclosed in WC500109302 and www.drugbank.ca/drugs/DB06186 was provided by Alphamab Inc. (Suzhou, Jiangsu, China), and Lakepharma Inc. (San Francisco, Calif., USA). Clinically used drug was also used to validate the key results. Human IgG-Fc (no azide) was bulk ordered from Athens Research and Technology (Athens, Ga., USA). Anti-mouse PD-1 mAb RMP1-14 was purchased from Bio-X Cell, Inc. (West Lebanon, N H, USA). Endotoxin levels of all mAbs were determined by LAL assay (Sigma) and were lower than 0.02 EU/n.

Tumor Growth and Regression Assay

Mice with either heterozygous or homozygous knock-in of human CTLA4 gene were challenged with given numbers of either colorectal cancer cell MC38, CT26 or melanoma cell line B16-F10. Immunotherapies were initiated at 2, 7 or 11 days after injection of tumor cells with indicated doses. The tumor growth and regression were determined using volume as the readout. The volumes (V) were calculated using the following formula.

V=ab²/2, where a is the long diameter, while b is the short diameter.

Humanization of L3D10

The L3D10 antibody was humanized by Lakepharma, Inc. through a service contract. The first humanized chain for each utilizes a first framework and contains the most human sequence with minimal parental antibody framework sequence (Humanized HC 1 and LC 1). The second humanized chain for each uses the same framework as HC 1 and LC 1 but contains additional parental L3D10 antibody sequences (Humanized HC 2 and LC 2). The third humanized chain for each utilizes a second framework and, similar to HC 2/LC 2, also contains additional parental sequences fused with the human framework (Humanized HC 3 and LC 3). The 3 light and 3 heavy humanized chains were then combined in all possible combinations to create 9 variant humanized antibodies that were tested for their expression level and antigen binding affinity to identify antibodies that perform similar to the parental L3D10 antibody.

Complete Blood Counts

Blood samples (50 μl) were collected at the age of 41 days using tubes with K₂EDTA (BD) and analyzed by HEMAVET HV950 (Drew Scientific Group, Miami Lakes, Fla., USA) following the manufacture's manual.

Histopathology Analysis of Internal Organ

H&E sections were prepared from formalin fixed organs harvested from mice that received therapeutic or control antibodies and were scored double blind. Score criteria: heart, infiltration in pericardium, right or left atrium, base of aorta, and left or right ventricle each count as 1 point; lung scoring is based on lymphocyte aggregates surrounding bronchiole, 1 stands for 1-3 small foci of lymphocyte aggregates per section, 2 stands for 4-10 small foci or 1-3 intermediate foci, 3 stands for more than 4 intermediate or presence of large foci, 4 stands for marked interstitial fibrosis in parenchyma and large foci of lymphocyte aggregates; liver scoring is based on lymphocyte infiltrate aggregates surrounding portal triad, 1 stands for 1-3 small foci of lymphocyte aggregates per section, 2 stands for 4-10 small foci or 1-3 intermediate foci, 3 stands for 4 or more intermediate or the presence of large foci, 4 stands for marked interstitial fibrosis in parenchyma and large foci of lymphocyte aggregates; kidney scoring: 1. Mild increase of glomerular cellularity; 2. Increase of glomerular cellularity and lymphocyte infiltration in distal or proximal tubes; 3. Large lymphocyte aggregates in collecting ducts; 4. Marked lymphocyte aggregates within cortex and medulla of kidney. Salivary gland scoring is based on lymphocyte infiltration in submandibular gland: 1 stands for 1-3 small foci of lymphocyte aggregates per section, 2 stands for 4-10 small foci or 1-3 intermediate foci, 3 stands for 4 or more intermediate or presence of large foci, 4 stands for marked interstitial fibrosis and tissue destruction in parenchyma and large foci of lymphocyte aggregates. Data shown are combined scores of all organs examined.

Analysis of Autoreactive T Cells Through F1 Intercross

As diagrammed in FIG. 31A, the C57BL/6.Ctla4^{h/h} mice were outcrossed to WT BALB/c mice. The F1 mice were intercrossed to generate the F2 in which both the Ctla4^{h} and H-2 alleles randomly segregated. The Ctla4 alleles and endogenous VSAg Mmtv8, 9 were genotyped using tail DNA according to published reports [24, 30], while the existence of H-2d haplotypes was determined by flow cytometry using peripheral blood leukocytes.

Clinical Chemistry for Drug Toxicity

The kit for measuring serum Troponin I Type 3, Cardiac (TNNI3) was purchased from Cloud-Clone Corp.(Cat. No. SEA478Mu), and TNNI3 levels were measured using ELISA following the manufacture's protocol. Creatinine levels were measured using Creatinine (serum) Colorimetric Assay Kit (Cayman Chemical) or Creatinine (CREA) Kit (RANDOX, Cat No, CR2336). Serum BUN levels were measured using UREA NITROGEN DIRECT kit (Stanbio laboratory) according to the manufacture's manual.

Biostatistics

The specific tests used to analyze each set of experiments are indicated in the figure legends. For each statistical analysis, appropriate tests were selected on the basis of whether the data with outlier deletion was normally distributed by using the D'Agostino & Pearson normality test. Data were analyzed using an unpaired two-tailed Student's t test or Mann-Whitney test to compare between two groups, one-way analysis of variance (ANOVA) or Kruskal-Wallis test for multiple comparisons, and two-way repeated-measures ANOVA for behavioral tests. Correlation coefficient and P-value of linear regression were calculated by Pearson's method. Sample sizes were chosen with adequate statistical power on the basis of the literature and past experience. No samples were excluded from the analysis, and experiments were not randomized except where specified. Blinding was not done during animal group allocation but was done for some measurements made in the study (i.e., tumor size measuring, scoring of histology). In the graphs, y-axis error bars represent S.E.M. or S.D. as indicated. Statistical calculations were performed using Excel (Microsoft), GraphPad Prism software (GraphPad Software, San Diego, Calif.) or R Software (www.r-project.org/). *P<0.05, **P<0.01, ***P<0.001.

Results

Human CTLA4 Knockin Mice Model Faithfully Recapitulates irAE of Combination Therapy

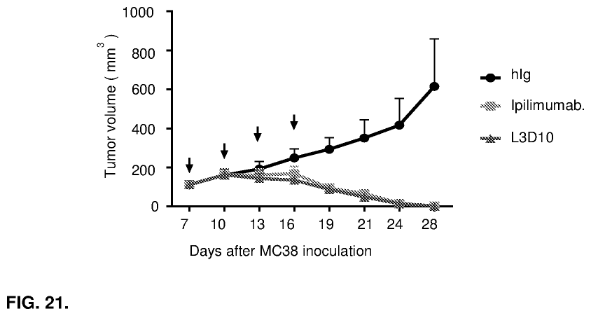

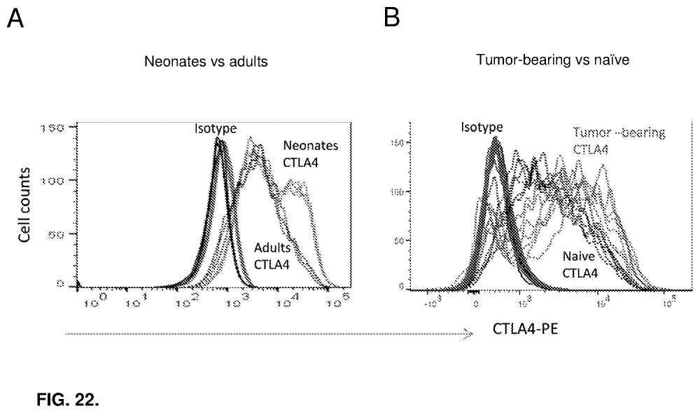

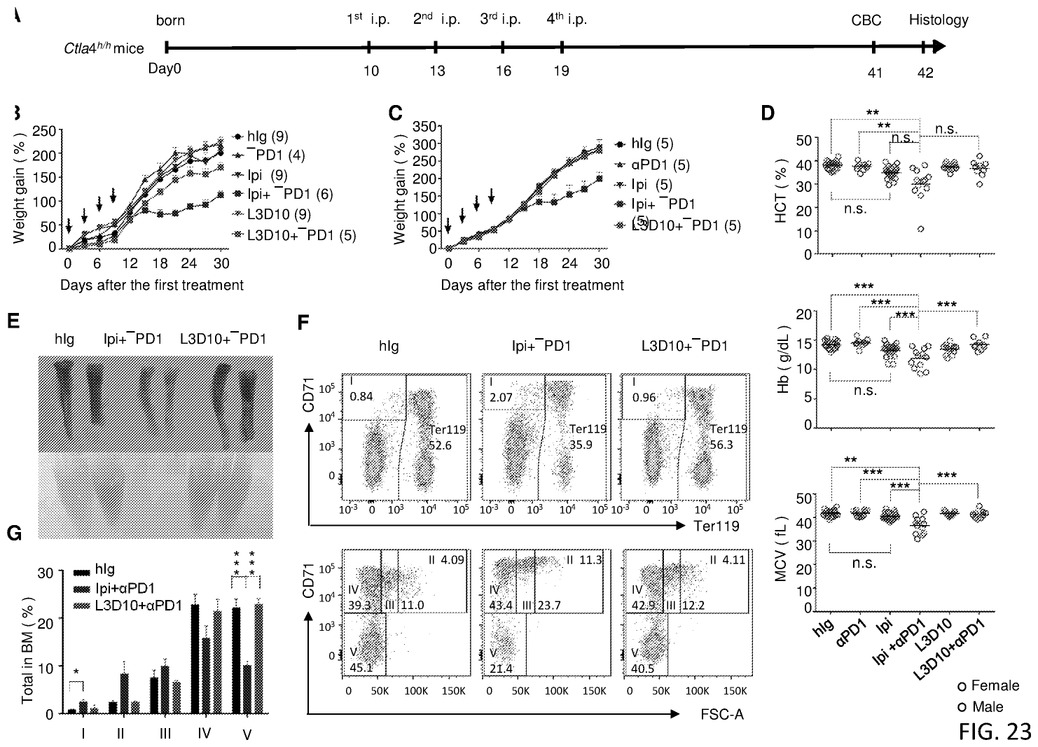

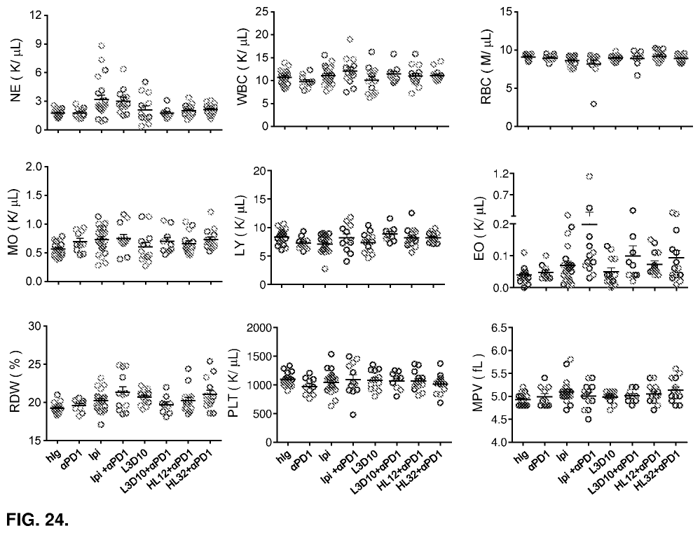

A major challenge in studying the mechanisms and preventive strategies of irAE in combination therapy is that the mouse tolerates high doses of anti-CTLA-4 mAb without significant AE. Two human CTLA-4 mAbs were selected for this study: the clinically used Ipilimumab and L3D10 that was the most potent among the panel of anti-CTLA-4 mAbs [24, 28]. When compared in the same model, the two mAbs were comparable in causing tumor rejection (FIG. 21). Since young mice expressed higher levels of CTLA-4, which recapitulated a feature in adult tumor-bearing mice (FIG. 22), human CTLA4 knockin (Ctla4^{h/h}) mice respectively with control human IgG-Fc, anti-CTLA-4 mAb Ipilimumab, L3D10, anti-PD-1, anti-PD-1+ Ipilimumab or anti-PD-1+L3D10 were treated during the perinatal period. The mice were treated on days 10, 13, 16 and 19 after birth, at the doses of 100 μg/mouse/injection, and were evaluated for the rate of body weight gain over time, and for hematologic and histopathology alterations at 6 weeks of age (FIG. 23A). As shown in FIG. 23B, while a combination of Ipilimumab and anti-PD-1 significantly retarded growth in female mice, either antibody alone did not have a major impact on body weight gain. Male mice also showed substantial and statistically significant growth retardation in response to anti-PD-1+ Ipilimumab (FIG. 23C). Remarkably, no growth retardation was observed when anti-PD-1+L3D10 was used (FIGS. 23B and 1C). To study the impact of combination therapy on hematopoiesis, total blood cell counts (CBC) were performed at one month after initiation of combination therapy (FIG. 24). Significant reduction of blood hematocrit (HCT), total hemoglobin (Hb) and Mean Corpuscular Volume (MCV) levels were observed among the majority of the mice treated with Ipilimumab+ anti-PD-1, while those that received L3D10+ anti-PD-1 were unaffected (FIG. 23D). The presentation of leukocytes was largely normal (FIG. 24). These data demonstrate that the combination of anti-PD-1 and Ipilimumab, but not that of anti-PD-1 and L3D10, causes anemia. As a single agent, Ipilimumab but not L3D10 induced anemia in high proportion of young mice, although the average reduction was not statistically significant. After necropsy, it is clear that red cell generation in the bone marrow was severely limited as the bones and the bone marrow flushed out from Ipilimumab+ anti-PD-1 treated mice appeared pale, while those of L3D10+ anti-PD-1 treated mice were comparable to those from the control IgG-treated mice (FIG. 23E). To quantitate the defects in the red cell lineage in the bone marrow, the distribution of CD71 and Ter119 markers among the bone marrow cells as well as the cell sizes were analyzed. These markers were used to mark five stages of erythrocyte development, sequentially from I to V: stage I, CD71⁺ Ter119⁻; stage II, FSC-A^{hi}CD71⁺ Ter119⁺; stage III, FSC-A^{mi}CD71⁺ Ter119⁺; stage IV, FSC-A^{lo}CD71⁺ Ter119⁺; and stage V, CD71⁻ Ter119⁺. As shown in FIG. 23F and FIG. 23G, anti-PD-1+ Ipilimumab-treated mice showed a significant increase of progenitor cells (stage I) and reduction in the frequency of mature red blood cells (stage V), which explains the apparent severe anemia. In contrast, L3D10 and anti-PD-1 treated mice exhibited normal distribution and maturation of erythrocytes in the bone marrow.

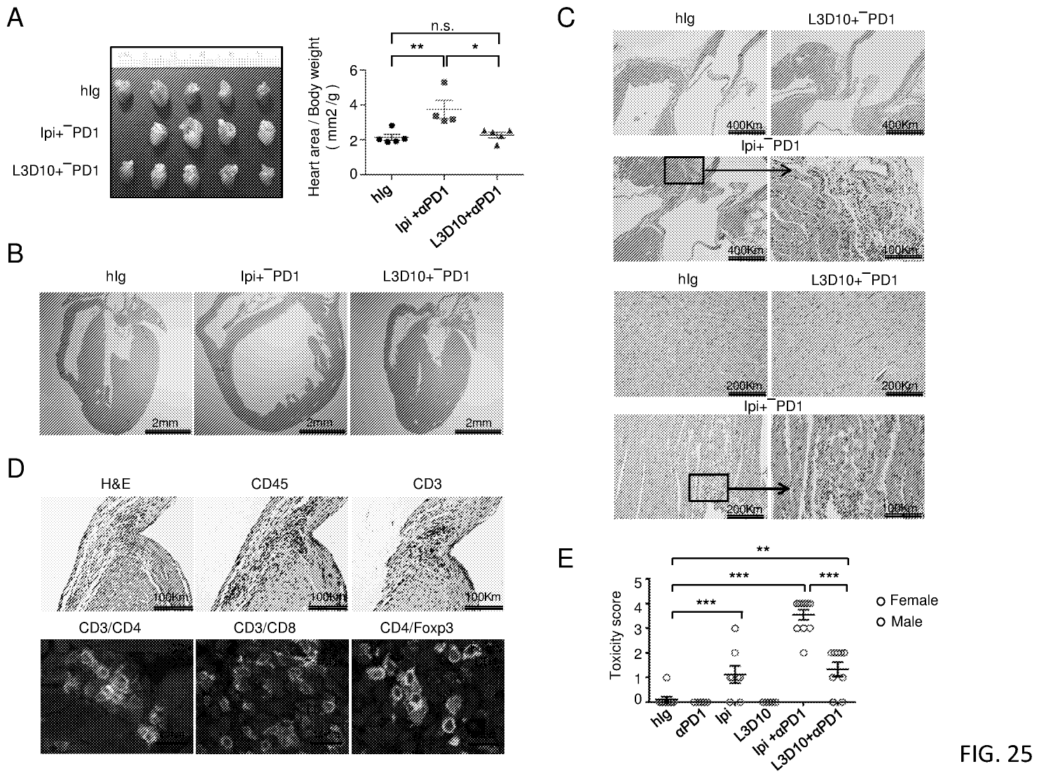

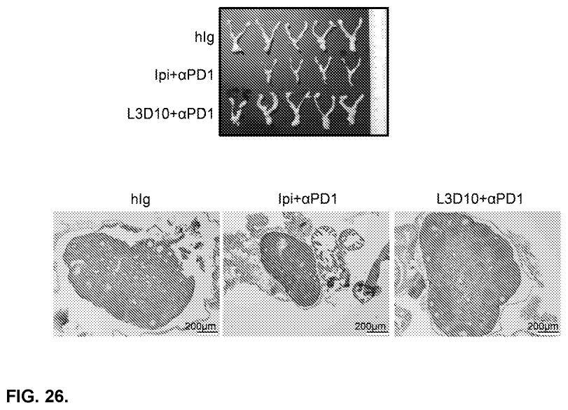

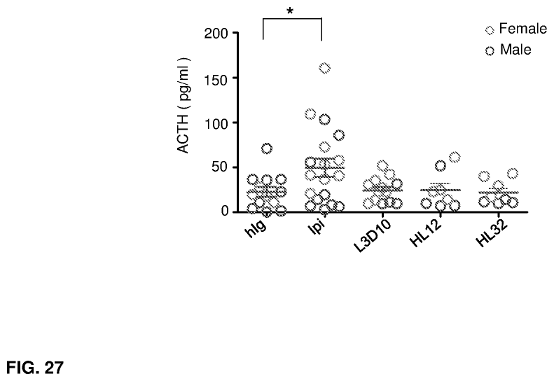

The dramatic difference in growth rate of CTLA4^{h/h} mice that received anti-PD-1 in conjunction with L3D10 vs Ipilimumab suggests that the two anti-CTLA-4 mAbs may induce very different AEs. To test this possibility, mice were sacrificed and necropsy was performed when they reached 42 days of age. Marked cardiomegaly was observed in anti-PD-1+ Ipilimumab-treated, but not in anti-PD-1+L3D10-treated mice (FIG. 25A). The enlarged heart showed dilation of chambers of both the right and left ventricles, albeit more conspicuous on left ventricle, indicating severe dilated cardiomyopathy. The left ventricular and ventricular sepal myocardium wall thickness decreased more than 50% in comparison with heart from the hIgG treated group (FIG. 25B). The histology demonstrated myocarditis with diffuse and massive lymphocyte infiltrations in the endocardium and myocardium, degeneration of cardiomyocytes and structural disruptions at the inflammatory foci (FIG. 25C). High abundance of CD45⁺ and CD3⁺ T cells were observed in the heart from anti-PD-1+ Ipilimumab-treated mice by immunohistochemistry (FIG. 25D, upper panels), consistent with a T-cell-mediated pathology. These cells included both CD4 and CD8 T subsets (FIG. 25D, bottom panels). Foxp3⁺CD4⁺ Treg cells were present at the inflammatory sites of anti-PD-1+ Ipilimumab-treated mice, which suggests that tissue destruction occurred despite the presence of Treg (FIG. 25D). Mild to moderate inflammation was observed in mice that received either L3D10+ anti-PD-1 combination therapy or Ipilimumab monotherapy. However, neither L3D10 nor anti-PD-1 monotherapy caused detectable inflammation (FIG. 25E). The fact that anti-PD-1 treatment failed to induce inflammation in heart may be attributed to the use of mice with the C57BL/6 background, since mice with the C57BL/6 background failed to develop heart diseases even when the Pdl gene was deleted [29], unlike the mice with the BALB/c background. Apart from heart, combination of Ipilimumab and anti-PD-1 mAb also induced severe defect in the female urinary-reproductive organs with histological findings of hypoplastic ovaries and uterus (FIG. 26). Consistent with defective adrenal gland function, a significant elevation of adrenocorticotropic hormone was observed, a likely response to defective production of cortisol by the adrenal gland (FIG. 27).

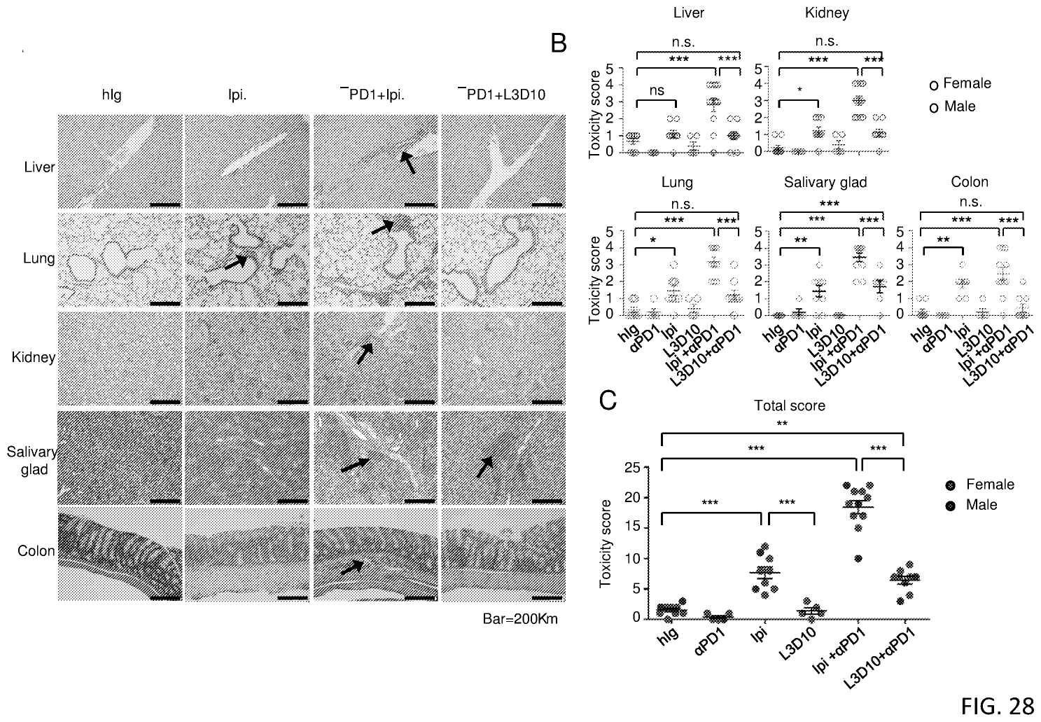

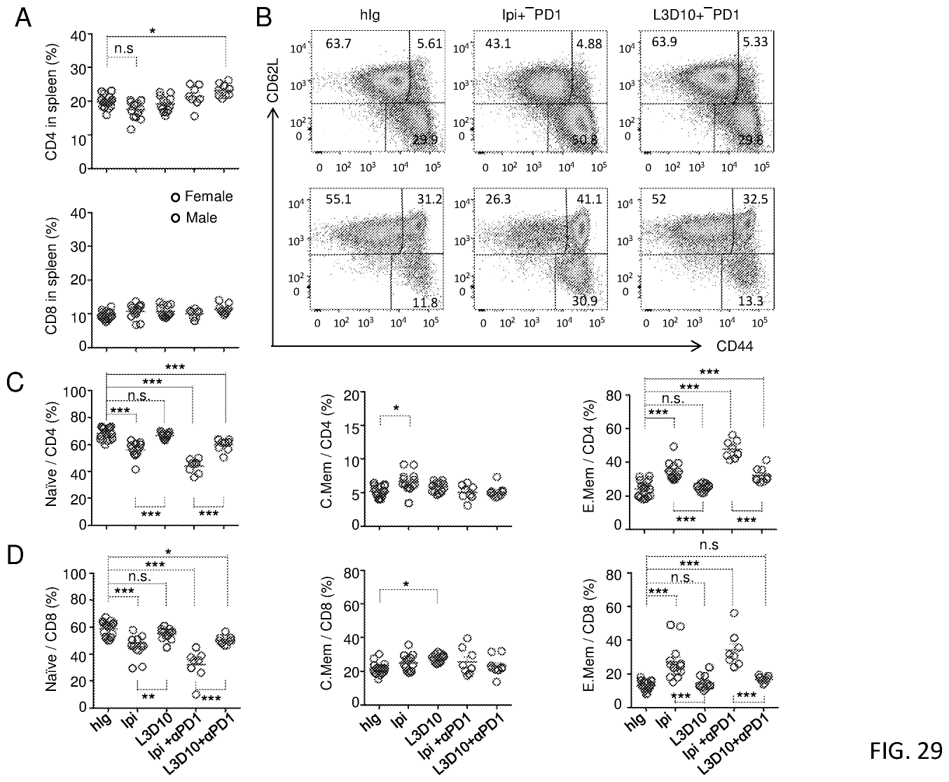

To quantitatively analyze the impact of anti-PD-1, and -CTLA-4 and their combinations on tissue destructions, histology analysis of internal organs and glands from mice receiving either control Ig or immunotherapeutic antibodies was performed. Organs and glands were fixed in 10% formalin, sectioned and stained with hematoxylin and eosin (H&E), and scored double blindly. Representative slides are shown in FIG. 28A, and the scores from individual mouse in each group are presented in FIG. 28B. The composite scores of all organs are presented in FIG. 28C. Confirming its safety, L3D10 monotherapy failed to induce severe inflammation in any organs examined. In contrast, moderate to severe inflammation was induced by Ipilimumab monotherapy in all mice, which is significantly stronger than occasional background inflammation in the control Ig, anti-PD-1 and L3D10 monotherapy groups. When combined with anti-PD-1, Ipilimumab induced inflammation in all mice, with severe inflammation found in all major organs. It is particularly noteworthy that transmural inflammation, which is the most severe form of histological findings in colons and a unique pathology feature of Crohn's disease, was observed in the anti-PD-1 and Ipilimumab-treated mice but was absent in other groups. When the scores from all organs were combined, it is clear that Ipilimumab+ anti-PD-1 induced dramatically stronger inflammation than L3D10+ anti-PD-1 treatment (FIG. 28C). In addition, Ipilimumab alone also induced significantly stronger adverse events than either anti-PD-1 alone or L3D10 alone as single agents (FIG. 28C).

Ipilimumab+Anti-PD1 but not L3D10+Anti-PD-1 Induces Systemic T Cell Activation and Expansion of Autoreactive Effector T Cells

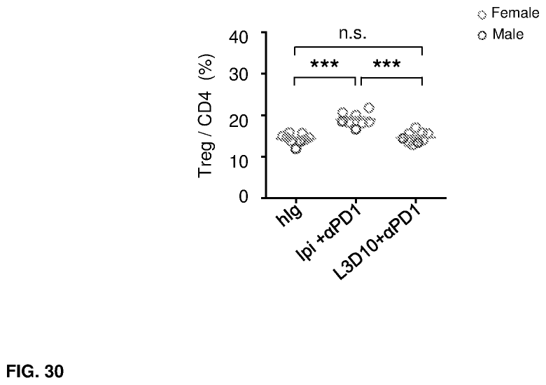

To understand the mechanisms of severe AEs induced by Ipilimumab+ anti-PD-1 combination therapy, the frequency and functional subsets of T cells in three groups of mice that received respectively control IgG, Ipilimumab+ anti-PD1 and L3D10+ anti-PD1 was analyzed. As shown in FIG. 29A, the frequencies of CD4 and CD8 T cells in three groups were substantially the same. Using CD44 and CD62L markers, a substantial expansion of effector memory T cells)(CD44^{hi}CD62L^{lo} in the Ipilimumab+ anti-PD-1 group was observed, although the frequency of central memory T cells (CD44^{hi}CD62L^{hi}) was unaffected (FIGS. 29B and 4D). Correspondingly, the frequency of naïve T cells was greatly reduced in anti-PD-1 and Ipilimumab-treated group (FIGS. 29B and 4D). The abnormal T cell activation was not due to depletion of Treg as the frequency of Treg was significantly elevated in the spleen (FIG. 30).

In order to understand the pathogenesis of irAE, it is of critical importance to understand the impact of immunotherapy on autoreactive T cells. To address this issue, the fact that endogenous self-antigens are recognized by a few selective Vβs was exploited [30]. Since C57BL/6 mice lack I-E to present endogenous superantigens, F2 mice were generated from a (B6.Ctla4^{h/h}×BALB/c WT) F1×F1 cross and the offspring were typed using mAbs that distinguish H-2^{d} (for BALB/c background) and H-2^{b} (for C57BL/6 background) haplotypes. PCR of tail DNA was also used to determine the status of mouse Ctla4 vs human CTLA4 alleles, as well as the endogenous VSAg8, 9 (FIG. 31A).

Using mice with targeted mutation of Ctla4, Yamaguchi et al. showed that, CTLA-4 helps to convert Vβ5, 11 and 12-expressing T cells into Treg as targeted mutation of CTLA-4 increased the % of Teff [31]. Therefore, the impact of anti-PD-1+ Ipilimumab or anti-PD-1+L3D10 on VSAg-reactive Teff and Treg in H-2^{d+} CTLA4^{h/h} mice was analyzed (FIG. 31B). Representative data using Vβ11, which reacts with VSAg8, 9, are shown in FIG. 31C, while summarized Treg/Teff ratios of VSAg-reactive T cells are shown in FIG. 31D. The data for specific Vβ are provided in supplemental Table 2.

TABLE 2
Ipilimumab induced preferential expansion of
Foxp3⁻ compartment among VSAg-reactive CD4 T cells

| | | Percentage of Vβs⁺ in CD4⁺FoxP3⁺ or CD4⁺FoxP3⁻(%) | | | |
|---|---|---|---|---|---|
| | | CD4⁺FoxP3⁻ | | | |
| Group | Mice ID | Vβ11⁺ | Vβ12⁺ | Vβ5⁺ | Vβ8⁺ |
| Ctla4^{h/h} | 6 | 1.64 | 0.14 | 0.04 | 36.00 |
| h/g | 24 | 1.84 | 0.27 | 0.13 | 28.80 |
| | 29 | 1.42 | 0.23 | 0.16 | 29.70 |
| | 32 | 1.50 | 0.18 | 0.06 | 29.50 |
| | 35 | 1.77 | 0.21 | 0.12 | 27.10 |
| | 42 | 2.05 | 0.14 | 0.31 | 35.60 |
| | MEAN ± SD | 1.70 ± 0.23 | 0.20 ± 0.05 | 0.14 ± 0.10 | 31.12 ± 3.74 |
| Ctla4^{h/h} | 41 | 1.44 | 0.23 | 0.10 | 29.80 |
| α-PD1 + L3D10 | 44 | 1.25 | 0.12 | 0.04 | 29.90 |
| | 45 | 1.93 | 0.22 | 0.08 | 34.20 |
| | 53 | 1.70 | 0.18 | 0.16 | 2 [symbol] .70 |
| | 65 | 1.59 | 0.28 | 0.07 | 32.10 |
| | 71 | 2.24 | 0.28 | 0.14 | 29.40 |
| | 7 [symbol] | 1.73 | 0.19 | 0.08 | 2 [symbol] .30 |
| | MEAN ± SD | 1.70 ± 0.32 | 0.21 ± 0.06 | 0.09 ± 0.04 | 30.20 ± 2.37 |
| Ctla4^{h/h} | 64 | 1.61 | 0.22 | 0.10 | 30.20 |
| [symbol] -PD1 + [symbol] | 73 | 2.99 | 0.47 | 0.15 | 28.50 |
| | 75 | 2. [symbol] 1 | 0.22 | 0.21 | 29.20 |
| | 81 | 2.82 | 0.30 | 0.23 | 28.00 |
| | 100 | 2.12 | 0.42 | 0.20 | 28.50 |
| | 101 | 3.10 | 0.52 | 0.44 | 35.00 |
| | MEAN ± SD | 2.52 ± 0.57** | 0.36 ± 0.1 [symbol] ** | 0.22 ± 0.12 | 23.20 ± 2.61 |
| Ctla4^{h/h} | 8 | 1.58 | 0.22 | 0.09 | 27.10 |
| α-PD1 + [symbol] | 17 | 1.11 | 0.16 | 0.09 | 26.80 |
| | 33 | 1.80 | 0.22 | 0.11 | 29.10 |
| | 34 | 1.53 | 0.19 | 0.08 | 28.10 |
| | 61 | 1. [symbol] 2 | 0.13 | 0.05 | 2 [symbol] .10 |
| | 63 | 2.45 | 0.17 | 0.11 | 33.00 |
| | MEAN ± SD | 1.63 ± 0.46 | 0.1 [symbol] ± 0.04 | 0.09 ± 0.02 | 28. [symbol] 7 ± 2.24 |

| | | Percentage of Vβs⁺ in CD4⁺FoxP3⁺ or CD4⁺FoxP3⁻(%) | | | |
|---|---|---|---|---|---|
| | | CD4⁺FoxP3⁺ | | | |
| Group | Mice ID | Vβ11⁺ | Vβ12⁺ | Vβ5⁺ | Vβ8⁺ |
| Ctla4^{h/h} | 6 | 3.79 | 1.26 | 0.74 | 33.20 |
| h/g | 24 | 3.53 | 1.52 | 1.41 | 27.60 |
| | 29 | 3.65 | 1.40 | 0.68 | 29.50 |
| | 32 | 3.41 | 1.31 | 0.82 | 28.70 |
| | 35 | 3.69 | 1.07 | 1.50 | 25.30 |
| | 42 | 3.45 | 1.27 | 0.82 | 33.20 |
| | MEAN ± SD | 3.59 ± 0.15 | 1.31 ± 0.15 | 1.00 ± 0.36 | 28.59 ± 3.14 |
| Ctla4^{h/h} | 41 | 2.69 | 1.39 | 0.68 | 28.60 |
| α-PD1 + L3D10 | 44 | 3.15 | 1.11 | 0.40 | 30.30 |
| | 45 | 3.82 | 0.95 | 0.41 | 34.50 |
| | 53 | 3.5 [symbol] | 0.96 | 0.80 | 29.00 |
| | 65 | 3.81 | 0.97 | 0.97 | 28.20 |
| | 71 | 3.6 [symbol] | 1.42 | 0.72 | 27.80 |
| | 7 [symbol] | 3.15 | 0.96 | 0.58 | 27.40 |
| | MEAN ± SD | 3.41 ± 0.42 | 1.11 ± 0.21 | 0.65 ± 0.21 | 29.40 ± 2.44 |
| Ctla4^{h/h} | 64 | 3.13 | 1.05 | 0.65 | 29.90 |
| [symbol] -PD1 + [symbol] | 73 | [symbol] | 2.11 | 0.81 | 26.40 |
| | 75 | 3.70 | 1.87 | 0.74 | 27.40 |
| | 81 | 3.85 | 0. [symbol] | 1.41 | 24.90 |
| | 100 | 3. [symbol] 1 | 1.08 | 1.14 | 26.30 |
| | 101 | 3.89 | 1.48 | [symbol] | 30.60 |
| | MEAN ± SD | 3.87 ± 0.64 | 1.41 ± 0.50 | 1.06 ± 0.39 | 27.58 ± 2.22 |
| Ctla4^{h/h} | 8 | 2.90 | 1.05 | 1.12 | 25.50 |
| α-PD1 + [symbol] | 17 | 2.44 | 0.91 | 0.74 | 23.70 |
| | 33 | 3.4 [symbol] | 1.35 | 0.93 | 28.90 |
| | 34 | 2.89 | 1.1 [symbol] | 0.82 | 27.30 |
| | 61 | 2.92 | 0.78 | 0.41 | 2 [symbol] .7 [symbol] |
| | 63 | 3.77 | 1.14 | 0.55 | 32.70 |
| | MEAN ± SD | 3.06 ± 0.47 | 1.07 ± 0.20 | 0.76 ± 0.26 | 27.80 ± 3.11 |

[symbol]

indicates data missing or illegible when filed

As shown in FIG. 31C, Ipilimumab+ anti-PD1 doubled the frequency of Foxp3⁻ Vβ11⁺CD4 T cells but increased that of the Foxp3⁺ Vβ11⁺CD4 T cells by merely 30%. Thus, Ipilimumab+ anti-PD-1 not only increased the frequency of autoreactive T cells, but also reduced the frequency of Treg among the autoreactive T cells. The frequency of non-VSAg-reactive T cells (Vβ8⁺) was unaffected regardless of Foxp3 expression. In contrast, anti-PD-1+L3D10 had no effect on frequency of CD4 T cells. The selective expansion of VSAg-reactive Teff was also observed among Vβ5⁺ and Vβ12⁺CD4 T cells (Table 2). As a result, Treg/Teff ratio among all studied VSAg-reactive CD4 T cells was significantly reduced in mice receiving anti-PD-1+ Ipilimumab (P=0.0026). The reduction was selective for VSAg-reactive T cells as the Treg/Teff ratio among Vβ8 was unaffected. These data demonstrate that antigen-specific suppression of autoreactive T cells is weakened by anti-PD-1+ Ipilimumab treatment. Furthermore, to address whether the treatment affected Treg/Teff during T cell development, the Treg/Teff ratio among VSAg-reactive thymocytes was also analyzed. As shown in FIG. 31E, anti-PD-1+ Ipilimumab had no impact on Treg/Teff ratio among thymocytes.

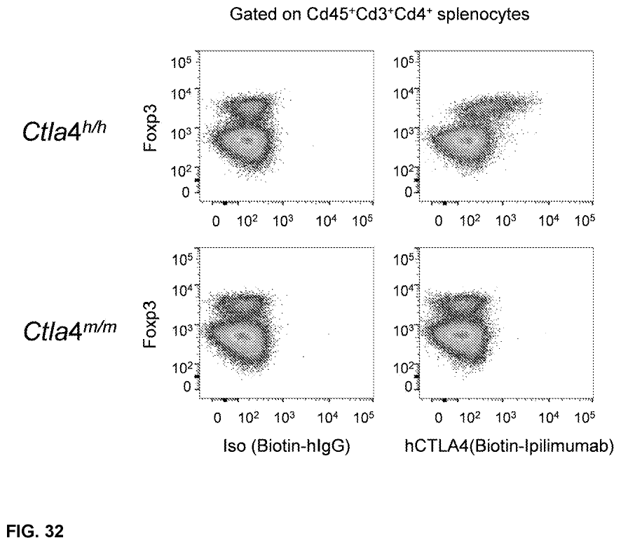

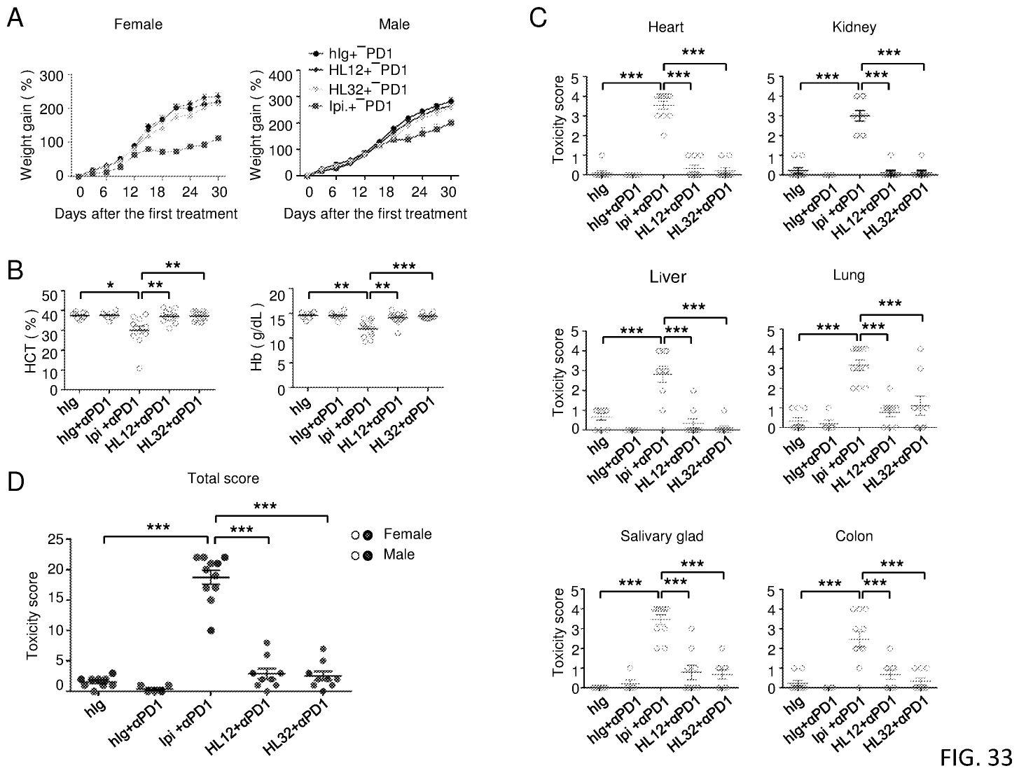

Anti-CTLA-4 mAbs used in this study react with human but not mouse CTLA-4 (FIG. 32) and thus cannot block the function of all CTLA-4 molecules in heterozygous mice carrying mouse Ctla4 and human CTLA4 alleles (Ctla4^{h/m}). It was tested if engaging a maximal of 50% of CTLA-4 is sufficient to cause reduced Treg/Teff among VSAg-reactive T cells. As summarized in FIG. 31D, in the CTLA4^{h/m} mice, no alteration in the ratio of conventional T cell over Treg was observed regardless of antibody treatment.

Humanized L3D10 Clones Exhibit Potent CITE but Minimal irAE

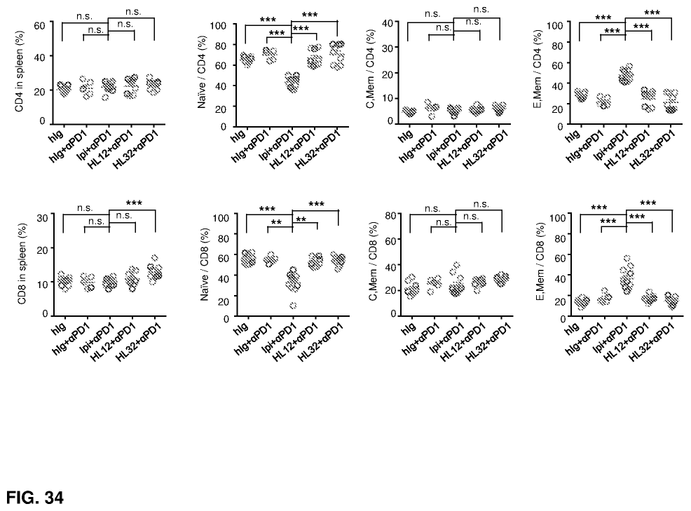

As the first step to translate the L3D10 antibody into clinical testing, L3D10 was humanized, producing two clones with comparable binding to CTLA-4, and these were compared to Ipilimumab for both irAE and CITE. As shown in FIG. 33A, in Ctla4^{h/h} mice, Ipilimumab but not HL12 and HL32 caused growth retardation when combined with anti-PD-1. In contrast to Ipilimumab, neither HL12 nor HL32 induced anemia as measured by HCT and Hb (FIG. 33B). Histopathology analyses further confirmed that when combined with anti-PD-1, HL12 and HL32 induced no inflammation in heart, liver, colon or kidney, although moderate inflammation in lung and salivary glands was observed in a small proportion of mice (FIG. 33C). The composite pathology scores revealed that HL12 and HL32 induced even less inflammation than L3D10 in combination therapy (when comparing FIG. 33D with FIG. 28C). Furthermore, no systemic activation of T cells was induced by the humanized clones when used in combination with anti-PD-1 antibody (FIG. 34). Therefore, the safety profile of L3D10 was not compromised during humanization.

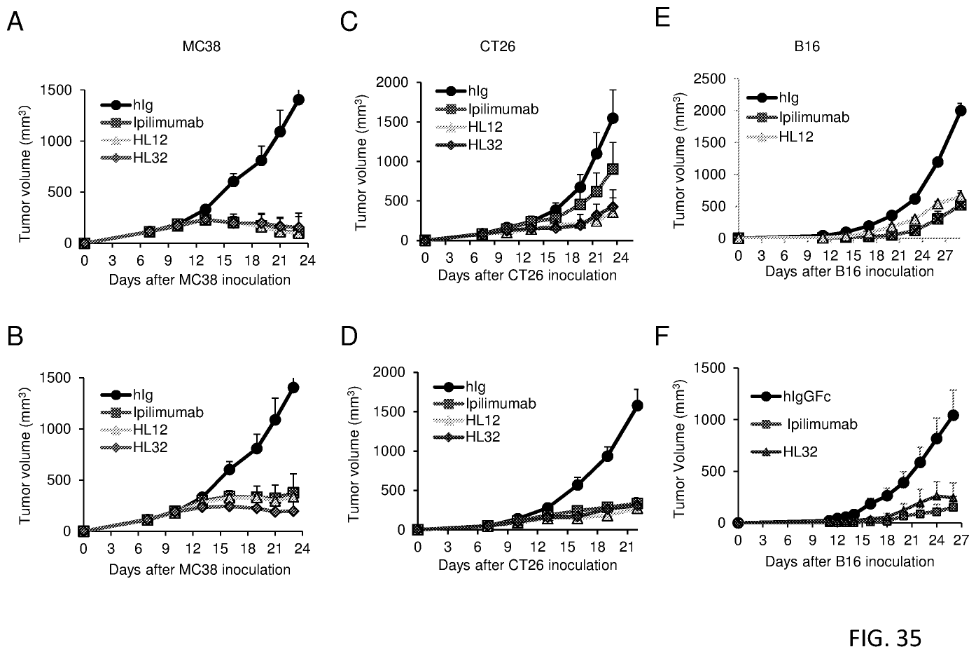

To determine whether better safety of HL12 and HL32 was achieved at the expense of therapeutic effect, Ipilimumab was first compared with HL12 and HL32 for their therapeutic effect. Previous studies have revealed that anti-murine CTLA-4 mAb monotherapy is capable of inducing rejection of colon cancer cell lines MC38 of C57BL/6 origin and CT26 of BALB/c origin. Thus F1 mice (Ctla4^{h/m}) were generated by crossing BALB/c.Ctla4^{m/m} mice and C57BL/6.Ctla4^{h/h} mice. As shown in FIG. 35, while MC38 tumors grow unimpeded in the control Ig-treated mice, their growth was prevented by adding low doses of anti-human CTLA-4 mAbs. At the dose of 30 μg/injection for 4 times, all anti-CTLA-4 mAbs were equally potent (FIG. 35A). At the low dose of 10 μg/injection for 4 times, HL32 appeared somewhat more potent than Ipilimumab and HL12, although the difference was not statistically significant (FIG. 35B). CT26 is somewhat more resistant than MC38 to anti-CTLA-4 immunotherapy, and thus requires higher doses. When high doses of antibodies were used, all three mAbs induced statistically significant growth inhibition (FIG. 35C). At a lower dose, Ipilimumab did reduce tumor growth somewhat, although the reduction was not statistically significant (P=0.29). On the other hand, both HL12 and HL32 induced clear growth inhibition (P<0.001) (FIG. 35D). Significant inhibitions were achieved by both antibodies when B16F10 melanoma tumor models were used (FIGS. 35E and 35F). Taken together, the humanized L3D10 clones HL12 and HL32 are at least as potent as Ipilimumab in causing tumor rejection. Therefore, the humanized mouse model allowed us to identify dramatically safer but at least equally potent anti-CTLA-4 mAbs.

In Ctla4^{h/m} Mice, Engagement of Human CTLA-4 is Sufficient for Inducing Tumor Rejection but not for Autoimmune Disease

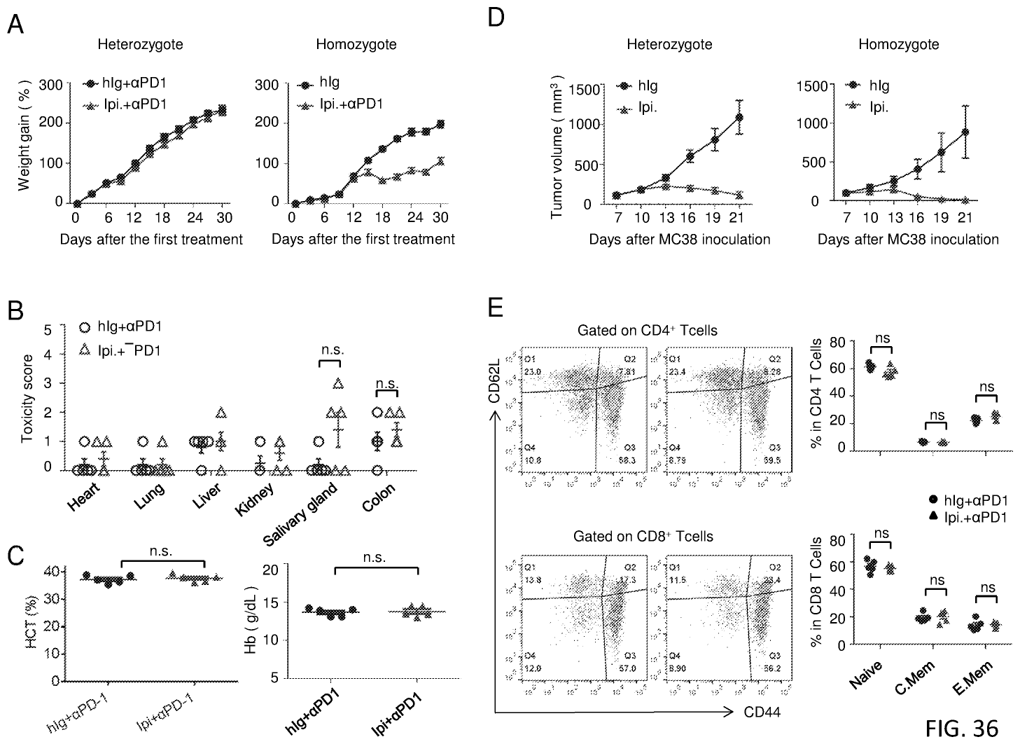

The above data that Ipilimumab can induce tumor rejection in CTLA4^{h/m} mice raised an intriguing issue as to whether this mAb can induce irAE by engaging only part of the cell surface CTLA-4. Since anti-human CTLA-4 mAbs used in this study do not react with mouse CTLA-4 molecules (FIG. 32), it was evaluated whether irAE and CITE require similar levels of receptor engagement by comparing irAE and CITE in the Ctla4^{h/m} mice. Surprisingly, the same dose of Ipilimumab+ anti-PD-1 that induced growth retardation in the Ctla4^{h/h} mice (FIG. 23A) failed to do so in the Ctla4^{h/m} mice (FIG. 36A), Consistent with the lack of irAE, histopathology analysis revealed that, with the exception of moderate inflammation in the salivary gland, anti-PD-1+ Ipilimumab did not cause inflammation in any other organs analyzed (FIG. 36B), even though the doses used caused severe inflammation in essentially all organs analyzed in homozygous mice (FIG. 25 and FIG. 28). Likewise, no anemia was observed in anti-PD-1+ Ipilimumab-treated Ctla4^{h/m} mice (FIG. 36C). Nevertheless, the heterozygous mice are nearly as responsive as the homozygous mice in respect to immunotherapy by Ipilimumab (FIG. 36D). Therefore irAE and cancer immunity can be uncoupled genetically: while the human CTLA4 gene confers CITE responses to Ipilimumab in a dominant fashion, its role in conferring irAE is recessive. These data also suggest distinct mechanisms responsible for irAE vs CITE.

In contrast to what was observed in homozygous mice (FIG. 29), the combination of Ipilimumab+ anti-PD-1 did not induce systemic activation of T cells in Ctla4^{h/m} mice (FIG. 36E). To understand the distinct autoimmune adverse effect, the impact of anti-PD-1+ Ipilimumab in human CTLA-4 homozygous and heterozygous mice on Treg/Teff ratio was analyzed. As shown in FIG. 31C and FIG. 31D, the same treatment that reduces Treg/Teff ratio in homozygous mice had no effect in the heterozygous mice. The distinct genetic requirement further strengthens the notion that autoimmune adverse effect can be uncoupled from cancer immunity. The lack of systemic T cell activation and failure to selectively expand autoreactive Teff explain the lack of irAE in Ctla4^{h/m} mice.

Observing irAE and CITE in the Same Setting

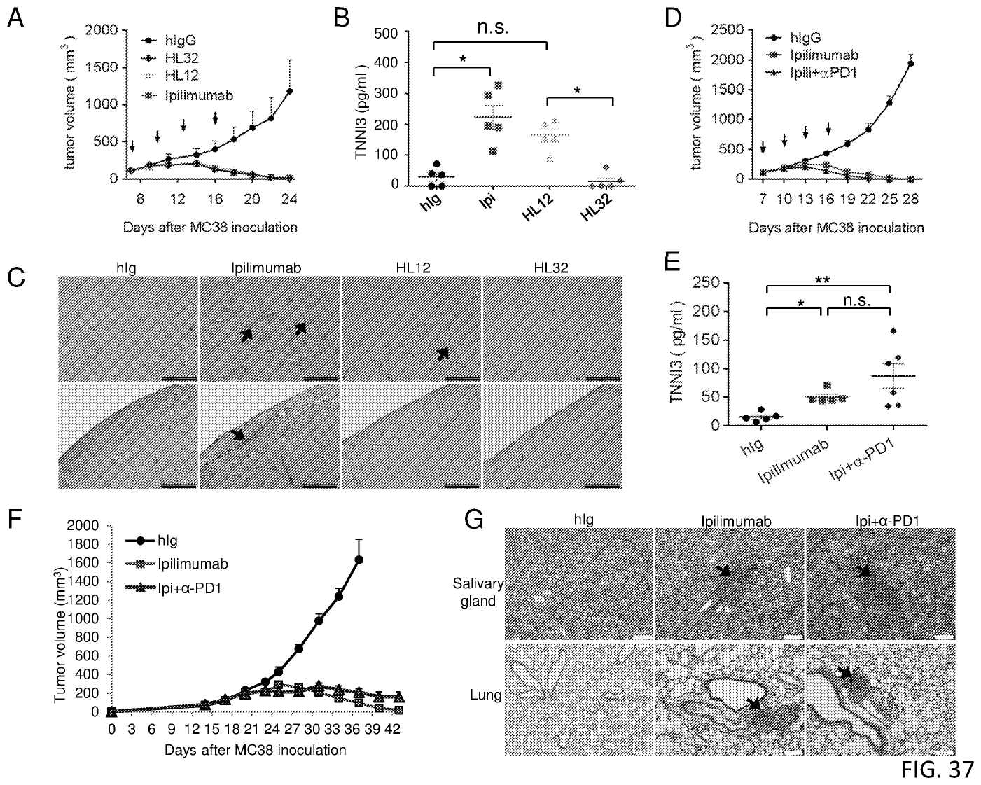

Although separate settings have been used so far to allow more robust evaluation of irAE and CITE, it is of interest to show irAE and CITE can be observed in the same setting. Two approaches were taken to achieve this goal. First, heart adverse events in young adult mice receiving anti-CTLA-4 antibody treatment were evaluated based on both cardiac troponin I (TNNI3, a routine diagnostic marker for various heart disorders) as serum marker and histology analysis. As shown in FIG. 37A, in 6-7 weeks young adult mice, anti-CTLA-4 mAbs induced similarly robust tumor rejection. Despite similar tumor rejections, the three mAbs induced distinct adverse heart defects. Notably, Ipilimumab induced high levels of TNNI3 (FIG. 37B). Correspondingly, histology analysis revealed extensive hyaline deposits within and outside myocytes (FIG. 37C, upper panel) with extensive pericardial inflammation (FIG. 37C, lower panel). Significant although lower levels of TNNI3 were observed in HL12-treated mouse sera, with correspondingly lower levels of hyalination and inflammation in the heart. In contrast, no elevation in serum TNNI3, and correspondingly histology findings of neither hyalination nor inflammation were observed in HL32-treated mice. When combined with anti-PD-1, therapeutic effect of Ipilimumab was comparable between monotherapy and combination therapy (FIG. 37D). While the heart toxicity was increased, there was no statistical significance between Ipilimumab alone group and Ipilimumab plus anti-PD-1 group due to high individual variations typical in toxicity studies (FIG. 37E).

Conversely, CITE was tested using 10-day-old mice as they were robust for evaluating irAE. As shown in FIG. 37F, MC38 tumors grew progressively after being transplanted into 10-day-old mice. Remarkably, the young mice were highly responsive to Ipilimumab both in tumor rejection and in induction of irAE, as demonstrated by rapid tumor regression (FIG. 37F) and pervasive server organ inflammation (FIG. 37G).

Systemic T Cell Activation Strongly Correlates with irAE

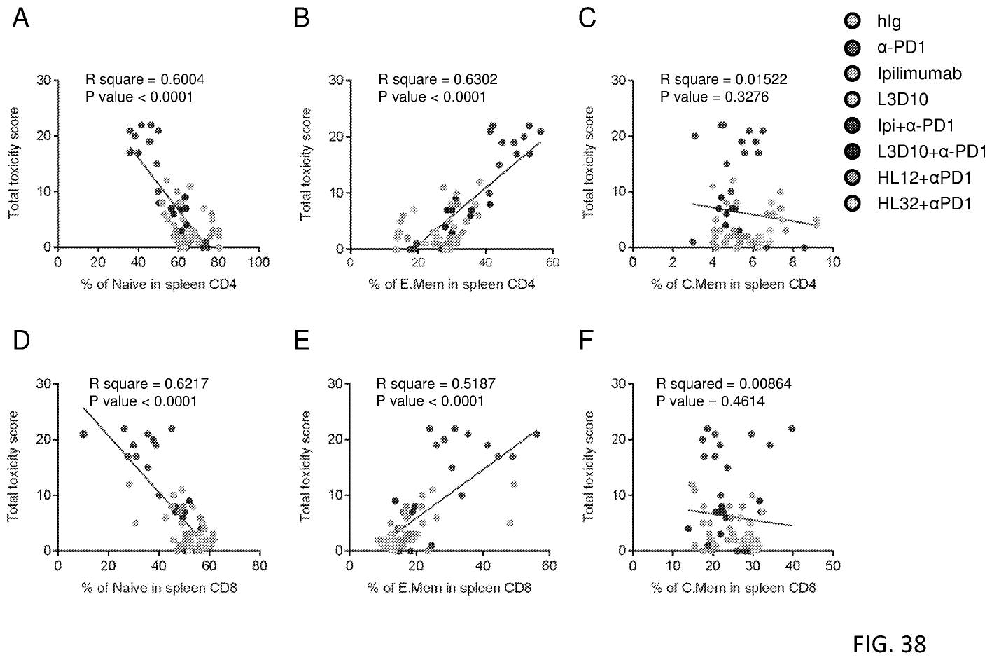

Since various antibodies used in this study demonstrate distinctive profiles of irAE and peripheral T cell activation, it is of interest to determine whether peripheral T cell activation correlates with irAE. As shown in FIGS. 38A and 38D, individual irAE scores of mice receiving either control or one of the five different anti-CTLA-4 mAbs administrations negatively correlate with the percentages of naïve CD4 and CD8 T cells in the spleen. Percentages of central memory T cells do not show such correlation (FIGS. 38C and 10F). In contrast, the percentage of effector memory T cells positively correlates with irAE (FIGS. 38B and 38E). The strong correlations suggest that pervasive T cell activation in the periphery is potentially the underlying cause for irAE.

Discussion

Since the description of irAE as a new clinical entity [10], there has been increasing interest in modeling the condition in mouse models in order to overcome this major bottleneck for the advancement of cancer immunotherapy. The progress has been slow, however, perhaps because mouse tumor models differ from human cancer patients whose immune system has had chronic interactions with the cancer tissue. In addition, since irAE may well be drug-specific, it is difficult to model the irAE of a specific anti-human CTLA-4 mAb with an anti-mouse CTLA-4 mAb. Our study here used human CTLA4 knockin mice to evaluate irAE of clinically used anti-CTLA-4 mAb. It was shown that this model successfully recapitulated most pathological observations associated with Ipilimumab, either alone or in combination with anti-PD-1 mAb, including severe inflammation to organs, such as heart, lung, liver, kidney and intestine. Rare diseases associated with Ipilimumab, such as pure red cell aplasia [19, 20], were also observed in this model.

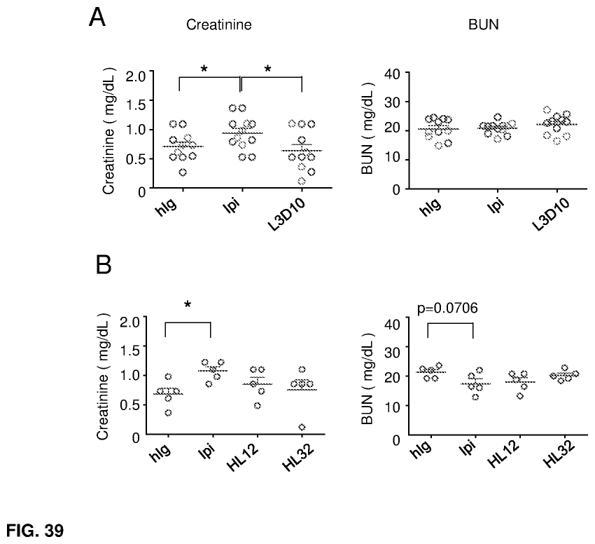

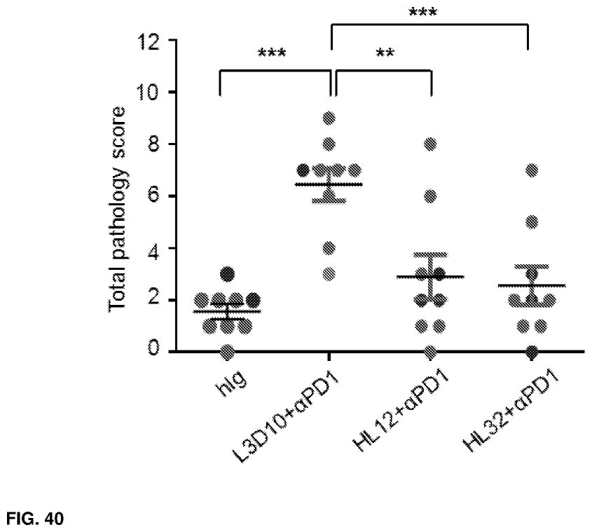

It should be noted that while the models can be used to mimic the combination of Ipilimumab and anti-PD-1, anti-PD-1 alone did not induce irAE in the model. Consistent with clinical observations, while Ipilimumab alone does induce significant adverse effects based on multiple organ inflammation, it is considerably less severe than combination therapy. Furthermore, in order to observe severe irAE, very young mice had to be used. However, while the adverse effect was less severe, laboratory and pathological findings of heart disease (FIG. 37) and kidney destruction (FIG. 39) were observed. It is of interest to note that of the two humanized L3D10 clones, one appears safer than the other in causing heart pathology. Overall, improved safety after humanization was observed without compromising efficacy (FIG. 40). Therefore, the Ctla4^{h/h} mice can be used to discriminate highly similar antibodies and thus to select subclones for further clinical development. A related study has demonstrated that humanization largely abrogated blocking activity of L3D10 without compromising either therapeutic effect or safety, further suggesting that neither CITE nor irAE relates to the blockade of CTLA-4-B7 interaction.

While very young mice are the best to evaluate irAE of anti-CTLA-4 mAbs, they also exhibit strong CITE after Ipilimumab treatment. Since many of the irAE, such as retarded growth, defective development of reproductive system, were observed in young mice, the model described herein may be valuable in predicting potential irAE that are uniquely important for pediatric cancer patients.

It is established that due to lymphopenia, T cells undergo extensive homeostatic proliferation in young mice [32, 33]. Since cancer patients and young mice are often lymphopenic, and lymphopenia is associated with homeostatic proliferation and autoimmune diseases [34, 35], it is of great interest to consider whether lymphopenia is a co-factor for the irAEs. If this is the case, one may use lymphopenia as a potential biomarker for irAE. Furthermore, the data demonstrated that tumor-bearing mice resemble young mice in expressing higher levels of Ctla4, therefore, data from young mice may shed light on that of tumor-bearing hosts. The spectrum of organ-inflammation, including cardiomyoditis, aplastic anemia, and endocrinopathy in the young mice recapitulates clinical findings and lends strong support for this thesis.

Liu et al. have recently used partial Treg depletion to sensitize mice for irAE [36]. While this model recapitulated some pathological features of irAE, it is of note that Ipilimumab systematically expands rather than depletes Treg in human cancer patients [37], a feature observed when Ipilimumab was used in human CTLA4 knockin mice (data not shown). For this reason, it is unlikely that a Treg-depletion-based model reflects the cause of irAE in cancer patients. Nevertheless, since it was found that combination therapy reduced the Treg/Teff ratios, a general defect in Treg may recapitulate some pathological features of irAE.

Using mice that are either homozygous or heterozygous for human CTLA4 alleles, irAE and CITE could be genetically uncoupled. Thus, while irAE is observed only in homozygous mice, CITE is observed in both heterozygous and homozygous mice. The marked difference in genetic requirement suggests distinct mechanisms for irAE and CITE: while irAE represents loss of CTLA-4 function imposed by Ipilimumab, CITE represents a gain of function of human CTLA-4 gene.

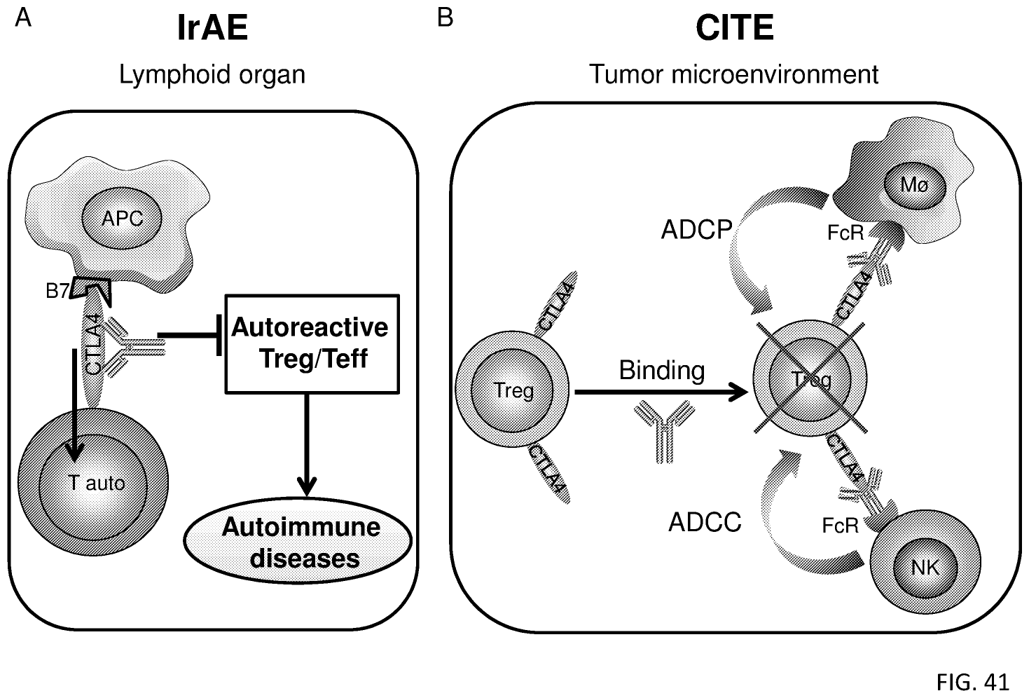

As immunological basis, the distinct genetic requirement is reflected on general T cell activation, as Ipilimumab+ anti-PD-1 induced extensive T cell activation in homozygous mice but not heterozygous mice. Using endogenous superantigen-reactivity as the marker for autoreactivity, it was found that Ipilimumab+ anti-PD-1 prevented conversion of autoreactive T cells into Treg, resulting in increased ratio of autoreactive effector cells over autoreactive Treg. Our previous studies demonstrated that Tregs are the most effective in suppressing T cell activation in vivo if they shared the antigen-specificity with the effector T cells [38]. Therefore, the increased ratio of autoreactive effector over auto-reactive Treg allowed activation of autoreactive T cells, leading to autoimmune diseases, as proposed in FIG. 41A.

It has been demonstrated that bi-allelic deletion of the CTLA4 gene reduced conversion of auto-reactive T cells into Treg [31]. The requirement for bi-allelic engagement by anti-CTLA4 mAbs for irAE is at least partially explained by the requirement for bi-allelic engagement of CTLA-4 in the conversion, as an increased ratio of autoreactive effector/regulatory T cells could lead to autoimmune diseases. The convergence between genetic inactivation of the Ctla4 locus and bi-allelic antibody engagement raised the intriguing possibility that Ipilimumab somehow inactivated the CTLA4 molecules. Since a related study demonstrated that Ipilimumab does not block B7-CTLA-4 interaction under physiological condition, the mechanism by which Ipilimumab inactivates CTLA-4 molecules remains to be determined.

Consistent with a dominant function of human CTLA-4 in CITE, several recent studies, including some by the inventors, have demonstrated a critical role for local depletion of Treg in tumor microenvironment. Thus, using anti-mouse CTLA-4 mAbs with identical Fv but distinct isotypes of Fc, Selby et al. demonstrated that the ability of anti-mouse CTLA-4 mAbs to induce tumor rejection is determined by the Fc portion [16]. Specifically, those with stronger affinity for activating FcgRs, including IgG2a and IgG2b can effectively induce tumor rejection and Treg depletion in the tumor microenvironment. In contrast, those with weaker affinity failed to do so. Consistent with this notion, Bulliard et al [18] showed that the Fcer1 gene, which encodes the activating signaling receptor subunit, is essential for anti-CTLA-4 mAb-induced tumor rejection. Furthermore, among the activating FcγRs that incorporate the Fcer1-encoded subunits, Simpson et al showed that tumor rejection and Treg depletion requires engagement of activating FcγRIV [17], suggesting an obligatory interaction between the Fc portion of anti-CTLA-4 mAb and FcR on either neutrophils or macrophages. Our data in the companion paper further demonstrates that anti-CTLA-4 induced tumor rejection requires Treg depletion but not blockade of B7-CTLA-4 interaction (FIG. 41B). Since the CTLA-4 mAbs were comparable in tumor rejection but yet vary greatly in inducing peripheral T cell activation, the data are inconsistent with the notion that anti-CTLA-4 antibodies promote tumor rejection by stimulating naïve T cell activation in the periphery¹. The distinct mechanism and locality associated with irAE and CITE provide us with new insights on producing more effective and safer CTLA-4-targeting reagents that favor Treg depletion within tumor microenvironment while avoid general T cell activation in the periphery lymphoid organ.

Classical checkpoint blockade hypothesis has suggested that anti-CTLA-4 mAb induces tumor rejection by inducing activation of naïve T cells in the lymphoid organ. In contrast, the data showed that actually the ability of mAbs to cause general activation of T cells in the lymphoid organ correlates with irAE rather than CITE. This is highlighted by the striking correlations between irAE score and systemic T cell activation triggered by combination therapy. In contrast, a related study demonstrated that Ipilimumab can induce tumor rejection without de novo priming of antigen-specific T cells. This is because at the time of Ipilimumab treatment, priming of T cells has already been achieved. At this point, release local suppression by Treg, rather than T cell priming in the lymphoid organ becomes the key to unleash cancer immunity.

Taken together, this work aims on addressing the fundamental issue that whether irAE and CITE can be uncoupled to allow development of safer and more effective immunotherapeutic antibodies. A new model that faithfully recapitulated irAEs is described herein, and using this model, it has been demonstrated that irAE and CITE are not inherently linked. This concept provides a foundation to identify therapeutic anti-CTLA-4 mAbs that are at least as effective as, but significantly less toxic than Ipilimumab. The data demonstrate that humanized L3D10 clones are potential candidates for therapeutic development for human cancer therapy. The notion that T cell activation in the tumor microenvironment entails cancer immunity, while general T cell activation in the peripheral lymphoid organs risks autoimmunity (FIG. 41) will likely have broad implications for selection of targets as well as the targeting therapeutic candidates.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### Example 3

### Antibody-Directed Lysosomal Degradation Underlies Immunotherapy-Related Adverse Effect of Anti-CTLA4 Monoclonal Antibodies

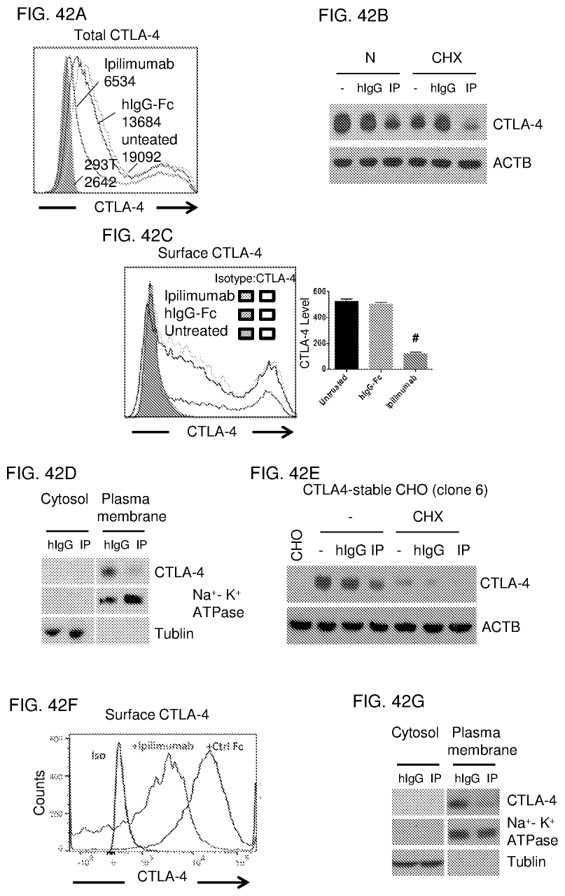

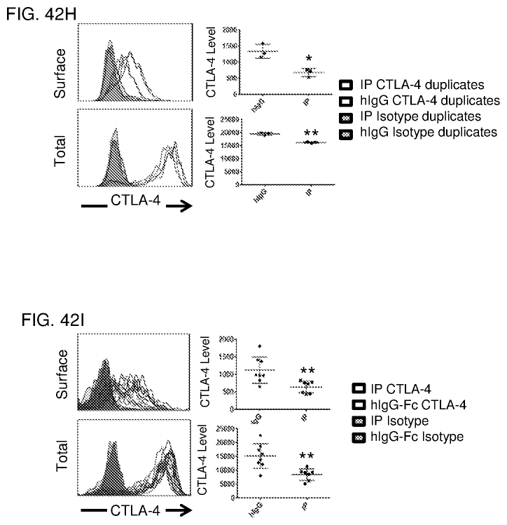

Given the strong autoimmune phenotype both in mice and human with targeted mutation of CTLA-4, it is proposed that irAE may relate to antibody-induced receptor down regulation. To test this hypothesis, multiple cell lines expressing exogenous human CTLA-4 molecules were generated and the impact of clinical drug Ipilimumab on CTLA-4 expression was tested. It was found that Ipilimumab induced the down-regulation of CTLA-4, especially cell surface CTLA-4, in both hCTLA-4-transfected 293T cells (FIG. 42A-D) and CHO stable cell lines expressing human CTLA-4 (FIG. 42E-G). Since CTLA4 primarily resides inside cells and recycles to the cell surface upon activation, cell surface expression of CTLA-4 may be a major factor governing the ability of Ipilimumab to induce CTLA-4 down-regulation. In untreated adult mice, very little cell surface CTLA-4 is detectable on regulatory T cells (data not shown). Correspondingly, Ipilimumab did not cause significant down regulation of CTLA-4 in naïve mice. In contrast, tumor-infiltrating Treg cells express considerably higher levels of cell surface CTLA-4, which is significantly down regulated by Ipilimumab both in vitro and in vivo (FIG. 42H-I).

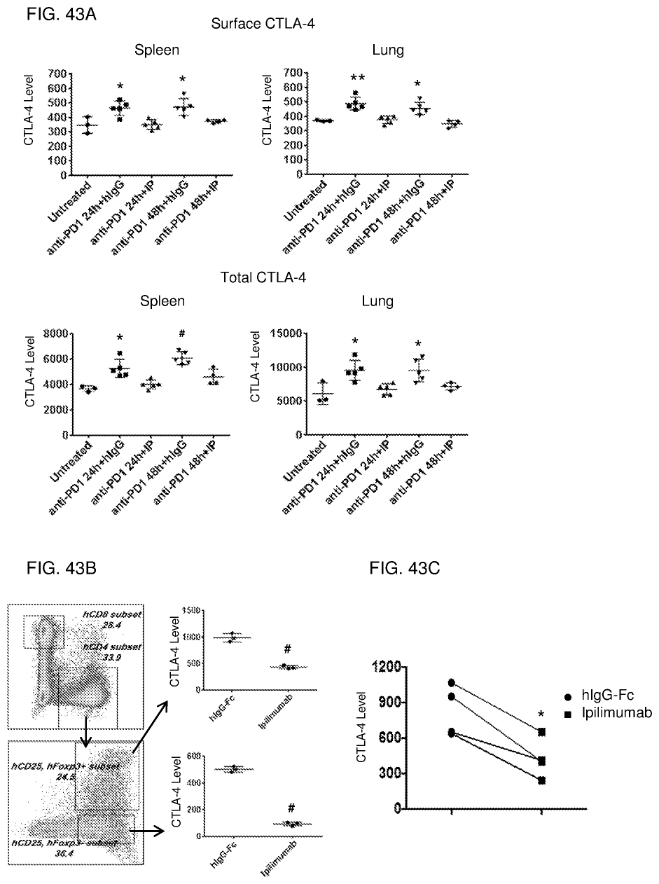

To test whether antibody-induced down regulation of cell surface CTLA4 contributes to susceptibility to irAE, cell surface CTLA-4 levels among Tregs were analyzed in an Ipilimumab-treated irAE CTLA-4^{h/h}-KI neonatal mouse model. In this model, Tregs have been shown to express considerably higher levels of surface CTLA-4 compared to adult mice, and Ipilimumab plus anti-PD-1 combination treatment causes severe irAE (18). Interestingly, it was found that with anti-PD-1 treatment, CTLA-4 expression was remarkably increased in lung and spleen Tregs (FIG. 43A). Combination treatment of Ipilimumab and anti-PD-1 caused significant down regulation of both surface and intracellular CTLA-4 back to the same level as in Tregs without treatment of anti-PD-1 (FIG. 43A). In addition, the down regulation of CTLA-4 by Ipilimumab in a human system was tested by stimulating peripheral blood mononuclear cells (PBMCs) from healthy donors' blood. Cell surface CTLA-4 levels were extremely low in non-activated human blood Tregs (Data not shown). Cell surface CTLA-4 was dramatically increased after activation by anti-CD3 and anti-CD28 stimulation, which was significantly down-regulated by Ipilimumab (FIG. 43B-C).

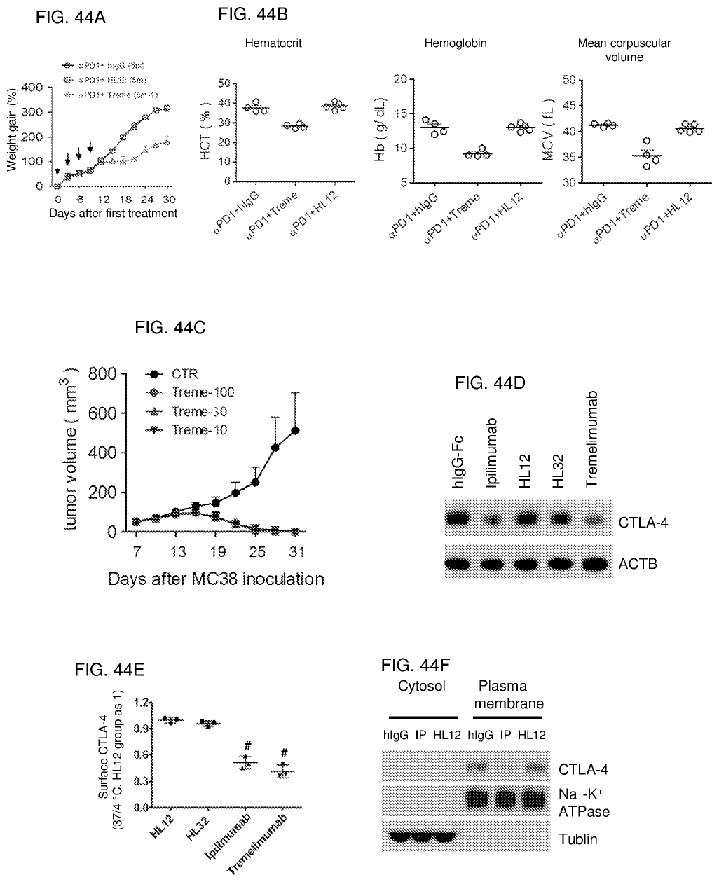

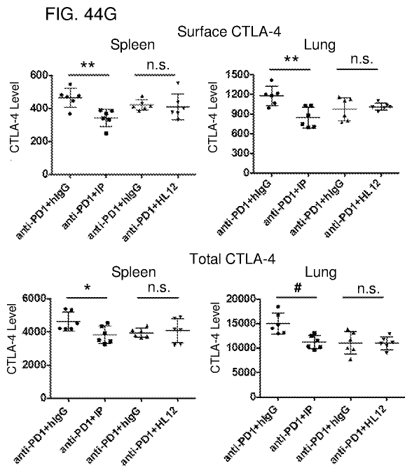

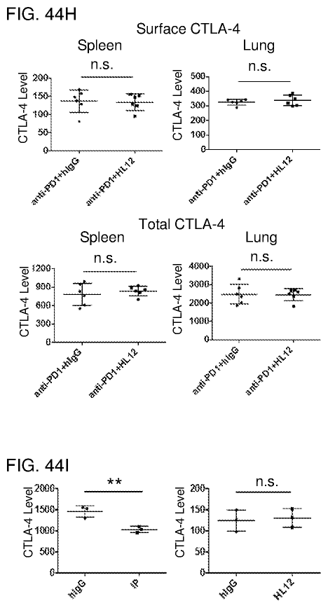

It has been shown that different anti-human CTLA-4 mAbs with comparable Cancer Immuno-Therapeutic Effect (CITE) lead to variety of irAE (18). The clinical drug Ipilimumab, but not human CTLA-4 mAbs HL12 and HL32, induced severe irAE in combination treatment with anti-PD-1 (18). An anti-CTLA-4 monoclonal IgG1 antibody generated with the same sequence of Tremelimumab also caused irAE in CTLA-4^{h/h}-KI neonatal mice model with CITE potential (FIG. 44A-C). Thus, the effects of CTLA4 down-regulation by these anti-CTLA-4 mAbs were compared. As shown in FIG. 44D-F, Ipilimumab and Tremelimumab (IgG1), but not HL12 and HL32, selectively down regulate surface and intracellular CTLA-4 in human cell lines expressing exogenous CTLA-4. In vivo study also showed that Ipilimumab, which triggered strong adverse effects, but not HL12, which did not cause any irAE, down-regulated surface and intracellular CTLA4 level of lung and spleen Tregs in an irAE CTLA-4^{h/h}-KI neonatal mouse model (FIG. 44G-H). Accordingly, similar results were shown in Ipilimumab and HL12 treated human activated Treg cells (FIG. 441). These data provide important evidence that antibody-induced down-regulation of surface CTLA-4 causes immunotherapy-related adverse effects.

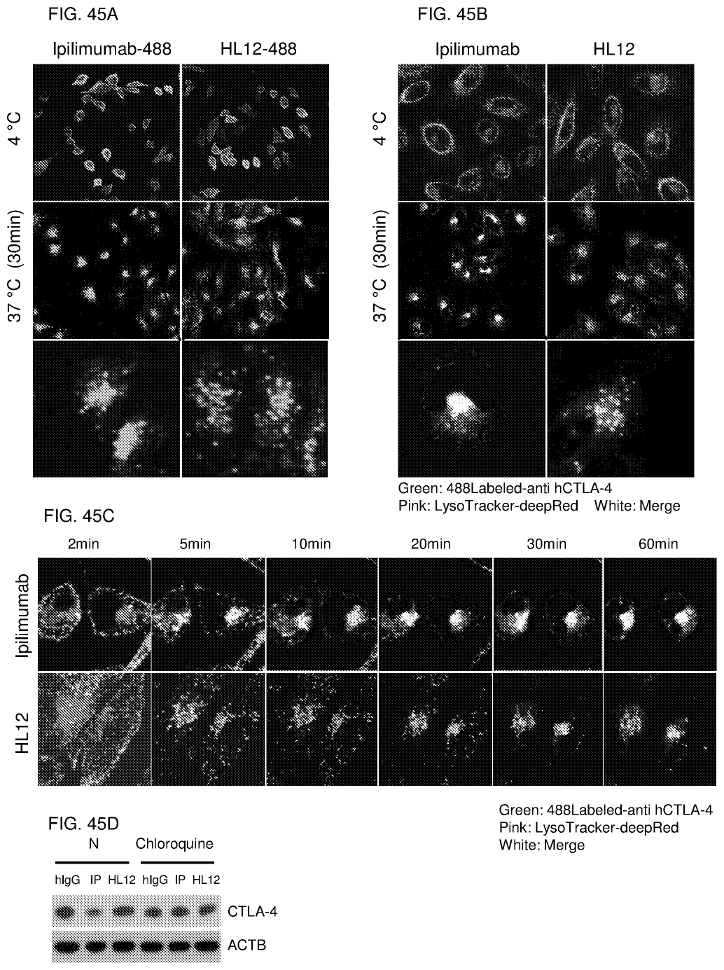

Since CTLA-4 is constitutively internalized from plasma membrane and undergoing both recycling and degradation (19), it was hypothesized that antibody-induced down-regulation of surface CTLA-4 may due to the lysosomal degradation of internalized surface CTLA-4. This was tested by labeling Ipilimumab and HL12 with Alex488 and tracking the surface CTLA-4 trafficking (FIG. 45A-C). Briefly, CTLA-4 expressing CHO cells were incubated with either Ipilimumab-Alex488 or HL12-Alex488 at 4° C., and surface CTLA-4 was shown to bind to the antibodies (FIG. 45A). After putting these cells back to 37° C., both Ipilimumab and HL12 labeled surface CTLA-4 were internalized. However, the patterns of internalized CTLA-4 localization were clearly different between Ipilimumab treatment and HL12 treatment (FIG. 45A). By staining cells with lysosome tracker, it was found that Ipilimumab, which caused significant down regulation of CTLA-4, but not HL12, which had no effects on CTLA-4 level, drove cell surface CTLA-4 to lysosome for degradation (FIG. 45B-C). Correspondingly, the down-regulation of CTLA-4 was inhibited by lysosome blockade in Ipilimumab treated cells, confirming the idea that Ipilimumab drives surface CTLA-4 degradation by lysosomes, but not HL12 (FIG. 45D).

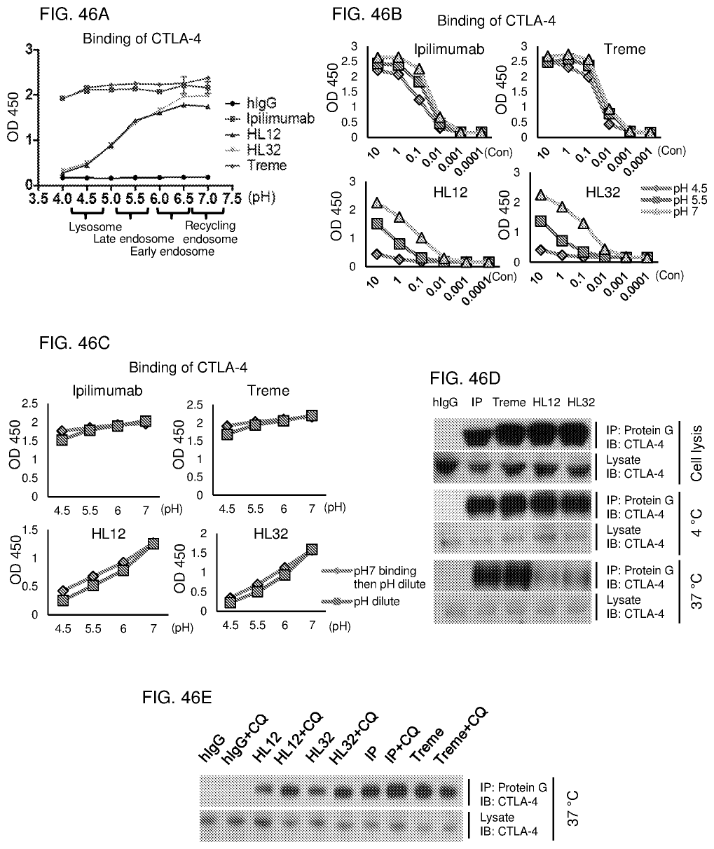

Cell surface proteins are targeted to early endosomes after being internalized. In endosomes, ligands may dissociate from their cognate receptors due to low pH, and the sustaining binding between ligands and receptors during endosome acidification is necessary for late lysosome degradation (20-22). Based on this, the fate of surface CTLA-4 going for lysosome degradation or recycling may be linked to their binding affinity with anti-CTLA-4 mAbs during endosome acidification. To test this, the CTLA4 binding of anti-CTLA-4 mAbs was compared in different pH conditions that exist during the process of endosome acidification. The data in FIG. 46A show that, at 10 μg/ml, Ipilimumab and Tremelimumab (IgG1) exhibit similar saturating binding from pH 7.0 to pH 4.0, which predicts that these complexes can be maintained at the cell surface (pH 7.0), endosomally (pH 5.0-6.5) or lysosomally (pH 4.5) pH (FIGS. 46A-B). In contrast, HL12 and HL32 started to lose the binding affinity with CTLA-4 when pH reached endosomal levels (pH less or equal to 6.0). As shown in FIG. 46B, Ipilimumab and Tremelimumab exhibit essentially identical dose response at pH 7.0 and pH 5.5. The amounts of antibodies needed at pH 5.5 to achieve 50% maximal pH 7.0 binding (IC₅₀) were essentially the same at those needed at pH7.0. The IC₅₀ at pH 4.5 was increased by approximately 50-250%. In contrast, HL12 and HL32 exhibit more than 10-fold reduction when binding at pH 5.5 was compared with that at pH7.0, based on increase of IC₅₀. The reduction of IC at pH 4.5 is greater than 100-fold reduction was observed when their binding at pH 4.5 was compared to pH 7.0, again based on increase of IC₅₀. Similar results of pH dependent binding were shown when the pH was decreased after CTLA-4 already binds to the antibodies (FIG. 46C). To establish whether loss of binding affinity in low pH links the dissociation between antibodies and CTLA-4 during internalization, surface CTLA-4 was labeled with anti-CTLA-4 mAbs at 4° C. before moving cells to 37° C. to allow CTLA-4 internalization and later either degradation or recycling back to the plasma membrane (FIGS. 46D-E). After incubation at 37° C., antibody-bound CTLA-4 was captured by protein-G beads and tested by western blot (FIGS. 46D-E). Data clearly showed that HL12 and HL32, but not Ipilimumab and Tremelimumab (IgG1), dissociated from CTLA-4 during antibody-induced CTLA-4 internalization (FIG. 46D), which was rescued by neutralizing pH during endosome-lysosome transportation (FIG. 46E).

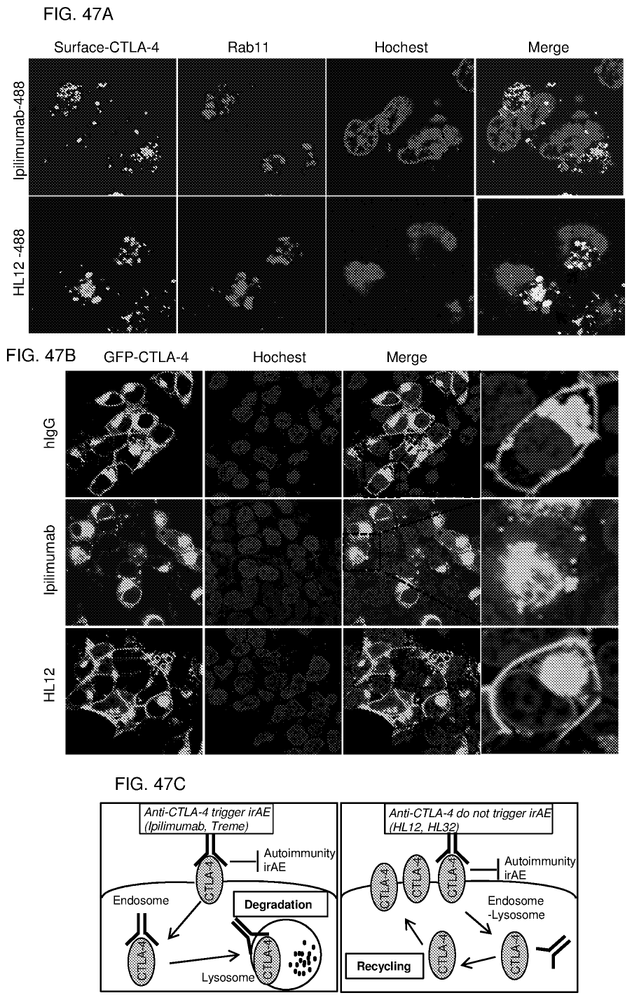

Since CTLA-4 internalized by HL12 and HL32 was released from antibodies and escaped from lysosome degradation, experiments were performed to test whether it could recycle back to the plasma membrane. By checking the recycling endosome marker Rab11, it was found that internalized CTLA-4 triggered by HL12 showed more co-localization with Rab11 compared to Ipilimumab treatment (FIG. 47A), indicating that internalized CTLA-4 triggered by HL12 but not Ipilimumab recycled back to cell surface. To confirm this, GFP-CTLA-4 transfected 293T cells were incubated with either control IgG, Ipilimumab or HL12, and cell surface CTLA-4 was tested by confocal microscopy (FIG. 47B). As expected, compared to the control hIgG-Fc treated cells, which have intact surface CTLA-4, Ipilimumab treated cells lost most of the cell surface CTLA-4 (FIG. 6B). Surface CTLA-4 has been shown in most of the HL12 treated cells, even though there were some gaps of the surface CTLA-4 in these cells, which may due to the unfinished recycling process (FIG. 47B).

The data demonstrate the important principles relevant to anti-CTLA-4 mAbs-induced irAE. As shown in FIG. 47C, anti-CTLA-4 mAbs with strong binding affinity of CTLA-4 at low pH, like Ipilimumab or Tremelimumab, will drive surface CTLA-4 to lysosomal degradation during internalization, which trigger irAE due to the loss of surface CTLA-4. In contrast, anti-CTLA-4 mAbs with weak binding affinity in low pH, like HL12 or HL32, will dissociate from CTLA-4 during antibody-induced internalization. Released surface CTLA-4 from these antibodies will recycle back to cell surface and maintain the function of CTLA-4 as a negative regulator of immune response. These findings provide important innovations to design novel anti-CTLA4 antibodies or engineering existing anti-CTLA-4 antibodies with better anti-tumor efficacy and lower toxicity.

### Example 4

### pH-Sensitive Anti-CTLA-4 Antibodies are More Effective in Treg Depletion in Tumor Microenvironment and Inducing Rejection of Large Established Tumors

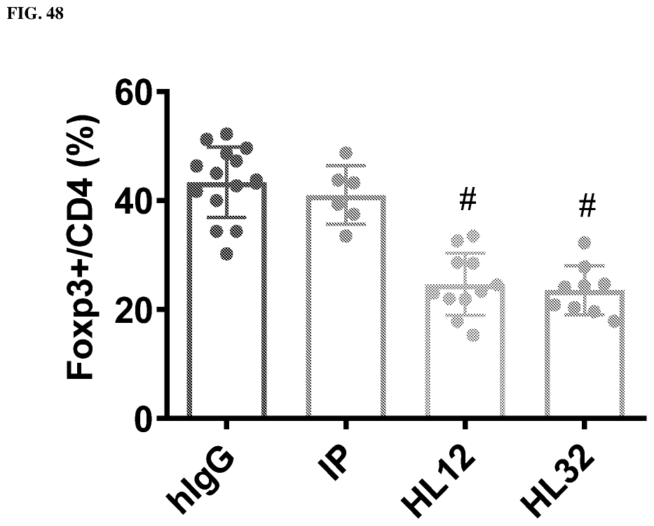

The key to pH-sensitive (non-irAE prone) anti-CTLA-4 antibodies is dissociation from CTLA-4 to allow its escape from lysosomal degradation and recycle to cell surface. The inventors realized that this property could help Treg depletion, as CTLA-4 levels determine target sensitivity to ADCC/ADCP. Given the essential role of Treg depletion in tumor microenvironment for CITE, it is of great interest to consider how the pH-sensitivity that confers less irAE would affect CITE. pH-sensitive and insensitive antibodies in Treg depletion were compared in tumor microenvironment and the rejection of large tumors. To test the function of the antibodies in Treg depletion in tumor microenvironment, the antibodies were injected into mice which were challenged with MC38 tumors 14 days previously. Sixteen hours later, the tumors were harvested and the % of Treg among CD4 T cells were assessed by flow cytometery. As shown in FIG. 48, while HL12 and HL32 significantly reduced Treg within 16 hours, Ipilimumab did not deplete Treg at this time point.

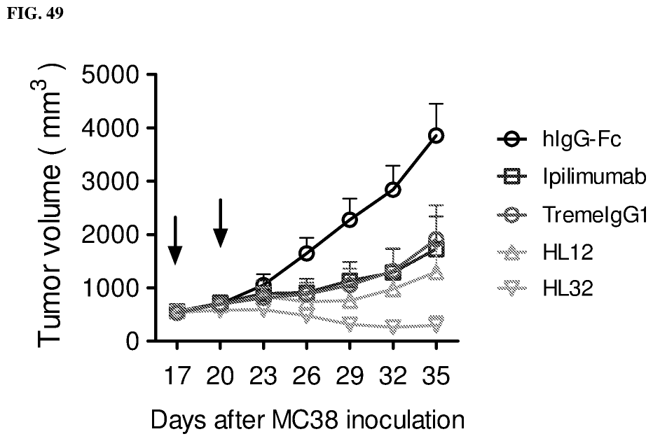

The inventors previously demonstrated that for various small tumors with four treatments, Ipilimumab, HL12 and HL32 are comparable in their efficacy in inducing tumor rejection (FIG. 35). Given the better efficacy of Treg depletion by HL12 and HL32, and given the critical rule for Treg in suppressing anti-tumor T cell responses, the efficacy of different anti-CTLA-4 antibodies were re-evaluated in a more challenging setting, i.e., in mice that bear large tumors, which have a well-established tumor microenvironment. Mice that have received MC38 tumors (with an average size of 10 mm in diameter) 17 days previously were treated twice with 1.5 mg/kg/dose of Ipilimumab, or HL12 and HL32. As shown in FIG. 49, at this relative low doses, HL32 was significantly more effective than Ipilimumab (P<0.0001). HL12 also showed a trend of better efficacy, although the difference did not reach statistical significance.

### REFERENCES CITED BEFORE EXAMPLES 2 AND 3

1 Korman A J, Peggs K S, Allison J P. Checkpoint blockade in cancer immunotherapy. Advances in immunology 2006; 90:297-339.
2 Hahn A W, Gill D M, Pal S K, Agarwal N. The future of immune checkpoint cancer therapy after PD-1 and CTLA-4. Immunotherapy 2017; 9:681-692.
3 Chen L, Han X. Anti-PD-1/PD-L1 therapy of human cancer: past, present, and future. The Journal of clinical investigation 2015; 125:3384-3391.
4 Leach D R, Krummel M F, Allison J P. Enhancement of antitumor immunity by CTLA-4 blockade [see comments]. Science 1996; 271:1734-1736.
5 Kocak E, Lute K, Chang X et al. Combination therapy with anti-CTL antigen-4 and anti-4-1BB antibodies enhances cancer immunity and reduces autoimmunity. Cancer Res 2006; 66:7276-7284.
6 Mokyr M B, Kalinichenko T, Gorelik L, Bluestone J A. Realization of the therapeutic potential of CTLA-4 blockade in low-dose chemotherapy-treated tumor-bearing mice. Cancer Res 1998; 58:5301-5304.
7 Hodi F S, O'Day S J, McDermott D F et al. Improved survival with ipilimumab in patients with metastatic melanoma. N Engl J Med 2010; 363:711-723.
8 Phan G Q, Yang J C, Sherry R et al. Cancer regression and autoimmunity induced by cytotoxic T lymphocyte-associated antigen-4 blockade in patients with metastatic melanoma. Proc Natl Acad Sci USA 2003; 100:8372-8377.
9 Selby M J, Engelhardt J J, Quigley M et al. Anti-CTLA-4 antibodies of IgG2a isotype enhance antitumor activity through reduction of intratumoral regulatory T cells. Cancer immunology research 2013; 1:32-42.
10 Simpson T R, Li F, Montalvo-Ortiz W et al. Fc-dependent depletion of tumor-infiltrating regulatory T cells co-defines the efficacy of anti-CTLA-4 therapy against melanoma. J Exp Med 2013; 210:1695-1710.
11 Bulliard Y, Jolicoeur R, Windman M et al. Activating Fc gamma receptors contribute to the antitumor activities of immunoregulatory receptor-targeting antibodies. The Journal of experimental medicine 2013; 210:1685-1693.
12 Qureshi O S, Zheng Y, Nakamura K et al. Trans-endocytosis of CD80 and CD86: a molecular basis for the cell-extrinsic function of CTLA-4. Science 2011; 332:600-603.
13 Walker L S, Sansom D M. The emerging role of CTLA4 as a cell-extrinsic regulator of T cell responses. Nat Rev Immunol 2011; 11:852-863.
14 Keler T, Halk E, Vitale L et al. Activity and safety of CTLA-4 blockade combined with vaccines in cynomolgus macaques. J Immunol 2003; 171:6251-6259.
15 May K F, Roychowdhury S, Bhatt D et al. Anti-human CTLA-4 monoclonal antibody promotes T cell expansion and immunity in a hu-PBL-SCID model: a new method for preclinical screening of costimulatory monoclonal antibodies. Blood 2005; 105:1114-1120.
16 He M, Chai Y, Qi J et al. Remarkably similar CTLA-4 binding properties of therapeutic ipilimumab and tremelimumab antibodies. Oncotarget 2017.
17 Linsley P S, Greene J L, Brady W, Bajorath J, Ledbetter J A, Peach R. Human B7-1 (CD80) and B7-2 (CD86) bind with similar avidities but distinct kinetics to CD28 and CTLA-4 receptors [published erratum appears in Immunity 1995 February; 2(2): following 203]. Immunity 1994; 1:793-801.
18 Stamper C C, Zhang Y, Tobin J F et al. Crystal structure of the B7-1/CTLA-4 complex that inhibits human immune responses. Nature 2001; 410:608-611.
19 Schwartz J C, Zhang X, Fedorov A A, Nathenson S G, Almo S C. Structural basis for co-stimulation by the human CTLA-4/B7-2 complex. Nature 2001; 410:604-608.
20 Wu Y, Guo Y, Huang A, Zheng P, Liu Y. CTLA-4-B7 interaction is sufficient to costimulate T cell clonal expansion. J Exp Med 1997; 185:1327-1335.
21 Linsley P S, Brady W, Urnes M, Grosmaire L S, Damle N K, Ledbetter J A. CTLA-4 is a second receptor for the B cell activation antigen B7. J Exp Med 1991; 174:561-569.
22 Ramarathinam L, Castle M, Wu Y, Liu Y. T cell costimulation by B7/BB1 induces CD8 T cell-dependent tumor rejection: an important role of B7/BB1 in the induction, recruitment, and effector function of antitumor T cells. J Exp Med 1994; 179:1205-1214.
23 Linsley P S, Bradshaw J, Greene J, Peach R, Bennett K L, Mittler R S. Intracellular trafficking of CTLA-4 and focal localization towards sites of TCR engagement. Immunity 1996; 4:535-543.
24 Bradshaw J D, Lu P, Leytze G et al. Interaction of the cytoplasmic tail of CTLA-4 (CD152) with a clathrin-associated protein is negatively regulated by tyrosine phosphorylation. Biochemistry 1997; 36:15975-15982.
25 Chuang E, Alegre M L, Duckett C S, Noel P J, Vander Heiden M G, Thompson C B. Interaction of CTLA-4 with the clathrin-associated protein AP50 results in ligand-independent endocytosis that limits cell surface expression. J Immunol 1997; 159:144-151.
26 Wing K, Onishi Y, Prieto-Martin P et al. CTLA-4 control over Foxp3+ regulatory T cell function. Science 2008; 322:271-275.
27 Hou T Z, Qureshi O S, Wang C J et al. A transendocytosis model of CTLA-4 function predicts its suppressive behavior on regulatory T cells. J Immunol 2015; 194:2148-2159.
28 Freeman G J, Borriello F, Hodes R J et al. Murine B7-2, an alternative CTLA4 counter-receptor that costimulates T cell proliferation and interleukin 2 production. J Exp Med 1993; 178:2185-2192.
29 Wu Y, Guo Y, Liu Y. A major costimulatory molecule on antigen-presenting cells, CTLA4 ligand A, is distinct from B7. J Exp Med 1993; 178:1789-1793.
30 Liu Y, Jones B, Brady W, Janeway C A, Jr., Linsley P S, Linley P S. Co-stimulation of murine CD4 T cell growth: cooperation between B7 and heat-stable antigen [published erratum appears in Eur J Immunol 1993 March; 23(3):780]. Eur J Immunol 1992; 22:2855-2859.
31 Punt J A, Havran W, Abe R, Sarin A, Singer A. T cell receptor (TCR)-induced death of immature CD4+CD8+ thymocytes by two distinct mechanisms differing in their requirement for CD28 costimulation: implications for negative selection in the thymus. J Exp Med 1997; 186:1911-1922.
32 Punt J A, Osborne B A, Takahama Y, Sharrow S O, Singer A. Negative selection of CD4+CD8+ thymocytes by T cell receptor-induced apoptosis requires a costimulatory signal that can be provided by CD28. J Exp Med 1994; 179:709-713.
33 Proietto A I, van Dommelen S, Zhou P et al. Dendritic cells in the thymus contribute to T-regulatory cell induction. Proc Natl Acad Sci USA 2008; 105:19869-19874.
34 Borriello F, Sethna M P, Boyd S D et al. B7-1 and B7-2 have overlapping, critical roles in immunoglobulin class switching and germinal center formation. Immunity 1997; 6:303-313.
35 Walunas T L, Lenschow D J, Bakker C Y et al. CTLA-4 can function as a negative regulator of T cell activation. Immunity 1994; 1:405-413.
36 Krummel M F, Allison J P. CD28 and CTLA-4 have opposing effects on the response of T cells to stimulation [see comments]. J Exp Med 1995; 182:459-465.
37 Ramagopal U A, Liu W, Garrett-Thomson S C et al. Structural basis for cancer immunotherapy by the first-in-class checkpoint inhibitor ipilimumab. Proceedings of the National Academy of Sciences of the United States of America 2017; 114:E4223-E4232.
38 Lute K D, May K F, Jr., Lu P et al. Human CTLA4 knock-in mice unravel the quantitative link between tumor immunity and autoimmunity induced by anti-CTLA-4 antibodies. Blood 2005; 106:3127-3133.
39 Hock B D, Starling G C, Patton W N et al. Identification of a circulating soluble form of CD80: levels in patients with hematological malignancies. Leuk Lymphoma 2004; 45:2111-2118.
40 Haile S T, Horn L A, Ostrand-Rosenberg S. A soluble form of CD80 enhances antitumor immunity by neutralizing programmed death ligand-1 and simultaneously providing costimulation. Cancer immunology research 2014; 2:610-615.
41 Lee K M, Chuang E, Griffin M et al. Molecular basis of T cell inactivation by CTLA-4 [In Process Citation]. Science 1998; 282:2263-2266.
42 Marengere L E, Waterhouse P, Duncan G S, Mittrucker H W, Feng G S, Mak T W. Regulation of T cell receptor signaling by tyrosine phosphatase SYP association with CTLA-4 [published errata appear in Science 1996 December 6; 274(5293)1597 and 1997 April 4; 276(5309):21]. Science 1996; 272:1170-1173.
43 Liu Y. Is CTLA-4 a negative regulator for T-cell activation? Immunol Today 1997; 18:569-572.
44 Tivol E A, Borriello F, Schweitzer A N, Lynch W P, Bluestone J A, Sharpe A H. Loss of CTLA-4 leads to massive lymphoproliferation and fatal multiorgan tissue destruction, revealing a critical negative regulatory role of CTLA-4. Immunity 1995; 3:541-547.
45 Waterhouse P, Penninger J M, Timms E et al. Lymphoproliferative disorders with early lethality in mice deficient in Ctla-4 [see comments]. Science 1995; 270:985-988.
46 Klocke K, Sakaguchi S, Holmdahl R, Wing K. Induction of autoimmune disease by deletion of CTLA-4 in mice in adulthood. Proc Natl Acad Sci USA 2016; 113:E2383-2392.
47 Bachmann M F, Kohler G, Ecabert B, Mak T W, Kopf M. Cutting edge: lymphoproliferative disease in the absence of CTLA-4 is not T cell autonomous. J Immunol 1999; 163:1128-1131.
48 Bachmann M F, Gallimore A, Jones E, Ecabert B, Acha-Orbea H, Kopf M. Normal pathogen-specific immune responses mounted by CTLA-4-deficient T cells: a paradigm reconsidered. Eur J Immunol 2001; 31:450-458.
49 Nguyen T V, Ke Y, Zhang E E, Feng G S. Conditional deletion of Shp2 tyrosine phosphatase in thymocytes suppresses both pre-TCR and TCR signals. J Immunol 2006; 177:5990-5996.
50 Liu X, Bal X F, Wen J et al. B7H Costimulates Clonal Expansion of, and Cognate Destruction of Tumor Cells by, CD8(+) T Lymphocytes In Vivo. J Exp Med 2001; 194:1339-1348.

### REFERENCES FOR EXAMPLE 2

1 Leach D R, Krummel M F, Allison J P. Enhancement of antitumor immunity by CTLA-4 blockade [see comments]. Science 1996; 271:1734-1736.
2 Kocak E, Lute K, Chang X et al. Combination therapy with anti-CTL antigen-4 and anti-4-1BB antibodies enhances cancer immunity and reduces autoimmunity. Cancer Res 2006; 66:7276-7284.
3 Mokyr M B, Kalinichenko T, Gorelik L, Bluestone J A. Realization of the therapeutic potential of CTLA-4 blockade in low-dose chemotherapy-treated tumor-bearing mice. Cancer Res 1998; 58:5301-5304.
4 Hodi F S, O'Day S J, McDermott D F et al. Improved survival with ipilimumab in patients with metastatic melanoma. N Engl J Med 2010; 363:711-723.
5 Phan G Q, Yang J C, Sherry R et al. Cancer regression and autoimmunity induced by cytotoxic T lymphocyte-associated antigen-4 blockade in patients with metastatic melanoma. Proc Natl Acad Sci USA 2003; 100:8372-8377.
6 Wolchok J D, Kluger H, Callahan M K et al. Nivolumab plus ipilimumab in advanced melanoma. N Engl J Med 2013; 369:122-133.
7 Larkin J, Chiarion-Sileni V, Gonzalez R et al. Combined Nivolumab and Ipilimumab or Monotherapy in Untreated Melanoma. N Engl J Med 2015; 373:23-34.
8 Hellmann M D, Rizvi N A, Goldman J W et al. Nivolumab plus ipilimumab as first-line treatment for advanced non-small-cell lung cancer (CheckMate 012): results of an open-label, phase 1, multicohort study. The Lancet Oncology 2017; 18:31-41.
9 Antonia S, Goldberg S B, Balmanoukian A et al. Safety and antitumour activity of durvalumab plus tremelimumab in non-small cell lung cancer: a multicentre, phase 1b study. The Lancet Oncology 2016; 17:299-308.
10 Fecher L A, Agarwala S S, Hodi F S, Weber J S. Ipilimumab and its toxicities: a multidisciplinary approach. Oncologist 2013; 18:733-743.
11 Ribas A, Kefford R, Marshall M A et al. Phase III randomized clinical trial comparing tremelimumab with standard-of-care chemotherapy in patients with advanced melanoma. Journal of Clinical Oncology 2013; 31:616-622.
12 Beer™, Kwon E D, Drake C G et al. Randomized, Double-Blind, Phase III Trial of Ipilimumab Versus Placebo in Asymptomatic or Minimally Symptomatic Patients With Metastatic Chemotherapy-Naive Castration-Resistant Prostate Cancer. Journal of clinical oncology: official journal of the American Society of Clinical Oncology 2017; 35:40-47.
13 Weber J, Mandala M, Del Vecchio M et al. Adjuvant Nivolumab versus Ipilimumab in Resected Stage III or IV Melanoma. N Engl J Med 2017.
14 Schadendorf D, Hodi F S, Robert C et al. Pooled Analysis of Long-Term Survival Data From Phase I I and Phase III Trials of Ipilimumab in Unresectable or Metastatic Melanoma. Journal of clinical oncology: official journal of the American Society of Clinical Oncology 2015; 33:1889-1894.
15 Korman A J, Peggs K S, Allison J P. Checkpoint blockade in cancer immunotherapy. Advances in immunology 2006; 90:297-339.
16 Selby M J, Engelhardt J J, Quigley M et al. Anti-CTLA-4 antibodies of IgG2a isotype enhance antitumor activity through reduction of intratumoral regulatory T cells. Cancer immunology research 2013; 1:32-42.
17 Simpson T R, Li F, Montalvo-Ortiz W et al. Fc-dependent depletion of tumor-infiltrating regulatory T cells co-defines the efficacy of anti-CTLA-4 therapy against melanoma. J Exp Med 2013; 210:1695-1710.
18 Bulliard Y, Jolicoeur R, Windman M et al. Activating Fc gamma receptors contribute to the antitumor activities of immunoregulatory receptor-targeting antibodies. The Journal of experimental medicine 2013; 210:1685-1693.
19 Nair R, Gheith S, Nair S G. Immunotherapy-Associated Hemolytic Anemia with Pure Red-Cell Aplasia. The New England journal of medicine 2016; 374:1096-1097.
20 Gordon I O, Wade T, Chin K, Dickstein J, Gajewski T F. Immune-mediated red cell aplasia after anti-CTLA-4 immunotherapy for metastatic melanoma. Cancer immunology, immunotherapy: CII 2009; 58:1351-1353.
21 Friedman C F, Proverbs-Singh T A, Postow M A. Treatment of the Immune-Related Adverse Effects of Immune Checkpoint Inhibitors: A Review. JAMA oncology 2016; 2:1346-1353.
22 Bertrand A, Kostine M, Barnetche T, Truchetet M E, Schaeverbeke T. Immune related adverse events associated with anti-CTLA-4 antibodies: systematic review and meta-analysis. BMC medicine 2015; 13:211.
23 Chen T W, Razak A R, Bedard P L, Siu L L, Hansen A R. A systematic review of immune-related adverse event reporting in clinical trials of immune checkpoint inhibitors. Annals of oncology: official journal of the European Society for Medical Oncology 2015; 26:1824-1829.
24 Lute K D, May K F, Lu P et al. Human CTLA-4-knock-in mice unravel the quantitative link between tumor immunity and autoimmunity induced by anti-CTLA-4 antibodies. Blood 2005.
25 Tivol E A, Borriello F, Schweitzer A N, Lynch W P, Bluestone J A, Sharpe A H. Loss of CTLA-4 leads to massive lymphoproliferation and fatal multiorgan tissue destruction, revealing a critical negative regulatory role of CTLA-4. Immunity 1995; 3:541-547.
26 Waterhouse P, Penninger J M, Timms E et al. Lymphoproliferative disorders with early lethality in mice deficient in Ctla-4 [see comments]. Science 1995; 270:985-988.
27 Klocke K, Sakaguchi S, Holmdahl R, Wing K. Induction of autoimmune disease by deletion of CTLA-4 in mice in adulthood. Proceedings of the National Academy of Sciences of the United States of America 2016; 113:E2383-2392.
28 May K F, Roychowdhury S, Bhatt D et al. Anti-human CTLA-4 monoclonal antibody promotes T cell expansion and immunity in a hu-PBL-SCID model: a new method for preclinical screening of costimulatory monoclonal antibodies. Blood 2005; 105:1114-1120.
29 Nishimura H, Okazaki T, Tanaka Y et al. Autoimmune dilated cardiomyopathy in PD-1 receptor-deficient mice. Science 2001; 291:319-322.
30 Abe R, Foo-Phillips M, Hodes R J. Genetic analysis of the Mls system. Formal Mls typing of the commonly used inbred strains. Immunogenetics 1991; 33:62-73.
31 Yamaguchi T, Kishi A, Osaki M et al. Construction of self-recognizing regulatory T cells from conventional T cells by controlling CTLA-4 and IL-2 expression. Proceedings of the National Academy of Sciences of the United States of America 2013; 110:E2116-2125.
32 Min B, Foucras G, Meier-Schellersheim M, Paul W E. Spontaneous proliferation, a response of naive CD4 T cells determined by the diversity of the memory cell repertoire. Proceedings of the National Academy of Sciences of the United States of America 2004; 101:3874-3879.
33 Min B, McHugh R, Sempowski G D, Mackall C, Foucras G, Paul W E. Neonates support lymphopenia-induced proliferation. Immunity 2003; 18:131-140.
34 King C, Ilic A, Koelsch K, Sarvetnick N. Homeostatic expansion of T cells during immune insufficiency generates autoimmunity. Cell 2004; 117:265-277.
35 Liu Y, Zheng P. CD24: a genetic checkpoint in T cell homeostasis and autoimmune diseases. Trends Immunol 2007; 28:315-320.
36 Liu J, Blake S J, Harjunpaa H et al. Assessing Immune-Related Adverse Events of Efficacious Combination Immunotherapies in Preclinical Models of Cancer. Cancer research 2016; 76:5288-5301.
37 Maker A V, Attia P, Rosenberg S A. Analysis of the cellular mechanism of antitumor responses and autoimmunity in patients treated with CTLA-4 blockade. Journal of Immunology 2005; 175:7746-7754.
38 Chang X, Zheng P, Liu Y. Selective Elimination of Autoreactive T cells in vivo by the Regulatory T Cells. Clinical Immunology 2009; 130:61-73.

### REFERENCES FOR EXAMPLE 3

1 Korman A J, Peggs K S, Allison J P. Checkpoint blockade in cancer immunotherapy. Advances in immunology 2006; 90:297-339.
2 Hodi F S, O'Day S J, McDermott D F, et al. Improved survival with ipilimumab in patients with metastatic melanoma. N Engl J Med 2010; 363(8):711-723.
3 Food and Drug Administration FDA approves new treatment for a type of late-stage skin cancer. Mar. 25, 2011.
4 Alexander W. The Checkpoint Immunotherapy Revolution: What Started as a Trickle Has Become a Flood, Despite Some Daunting Adverse Effects; New Drugs, Indications, and Combinations Continue to Emerge. PT 2016; March; 41(3):185-91.
5. Merck. Merck to present new data in five tumor types from studies evaluating pembrolizumab, the company's investigational anti-PD-1 antibody, at ESMO 2014. Sep. 2, 2014.
6. Food and Drug Administration FDA approves Keytruda for advanced melanoma. Sep. 4, 2014.
7 Wolchok J D, Kluger H, Callahan M K et al. Nivolumab plus ipilimumab in advanced melanoma. N Engl J Med 2013; 369:122-133.
8 Larkin J, Chiarion-Sileni V, Gonzalez R et al. Combined Nivolumab and Ipilimumab or Monotherapy in Untreated Melanoma. N Engl J Med 2015; 373:23-34.
9 Hellmann M D, Rizvi N A, Goldman J W et al. Nivolumab plus ipilimumab as first-line treatment for advanced non-small-cell lung cancer (CheckMate 012): results of an open-label, phase 1, multicohort study. The Lancet Oncology 2017; 18:31-41.
10 Antonia S, Goldberg S B, Balmanoukian A et al. Safety and antitumour activity of durvalumab plus tremelimumab in non-small cell lung cancer: a multicentre, phase 1b study. The Lancet Oncology 2016; 17:299-308.
11 Ribas A, Kefford R, Marshall M A et al. Phase III randomized clinical trial comparing tremelimumab with standard-of-care chemotherapy in patients with advanced melanoma. Journal of Clinical Oncology 2013; 31:616-622.
12 Beer™, Kwon E D, Drake C G et al. Randomized, Double-Blind, Phase III Trial of Ipilimumab Versus Placebo in Asymptomatic or Minimally Symptomatic Patients With Metastatic Chemotherapy-Naive Castration-Resistant Prostate Cancer. Journal of clinical oncology: official journal of the American Society of Clinical Oncology 2017; 35:40-47.
13 Wolchok J D1, Saenger Y. The mechanism of anti-CTLA-4 activity and the negative regulation of T-cell activation. Oncologist 2008; 13 Suppl 4:2-9.
14 Waterhouse P1, Penninger J M, Timms E et al. Lymphoproliferative disorders with early lethality in mice deficient in Ctla-4. Science 1995; 270(5238):985-8.
15 Klocke K1, Sakaguchi S2, Holmdahl R1 et al. Induction of autoimmune disease by deletion of CTLA-4 in mice in adulthood. Proc Natl Acad Sci USA 2016; 113(17):E2383-92.
16. Kuehn H S, Ouyang W, Lo B et al. Immune dysregulation in human subjects with heterozygous germline mutations in CTLA4. Science 2014; 345(6204):1623-1627.
17 Lo B, Zhang K, Lu W et al. Patients with LRBA deficiency show CTLA4 loss and immune dysregulation responsive to abatacept therapy. Science 2015; 349(6246):436-40.
18 Du X, Liu M, Su J et al. Uncoupling therapeutic from immunotherapy-related adverse effects for safer and effective anti-CTLA-4 antibodies in CTLA4 humanized mice. Cell Res 2018; Feb. 20.
19 Qureshi O S, Kaur S, Hou T Z et al. Constitutive clathrin-mediated endocytosis of CTLA-4 persists during T cell activation. J Biol Chem 2012; 287(12):9429-40.
20 Devanaboyina S C, Lynch S M, Ober R J et al. The effect of pH dependence of antibody-antigen interactions on subcellular trafficking dynamics. MAbs 2013; 5(6):851-9.
21 Igawa T, Ishii S, Tachibana T et al. Antibody recycling by engineered pH-dependent antigen binding improves the duration of antigen neutralization. Nat Biotechnol 2010:1203-7.
22 van Weert A W1, Geuze H J, Groothuis B et al. Primaquine interferes with membrane recycling from endosomes to the plasma membrane through a direct interaction with endosomes which does not involve neutralisation of endosomal pH nor osmotic swelling of endosomes. Eur J Cell Biol 2000; 79(6):394-9.

1. An anti-CTLA-4 antibody for use in treating cancer, wherein the antibody does not confer systemic T cell activation or preferential expansion of self-reactive T cells.

2. An anti-CTLA-4 antibody for use in treating cancer, wherein the antibody allows CTLA-4 to cycle back to a cell surface.

3. The anti-CTLA-4 antibody of claim 2, wherein the antibody binds to CTLA-4 with a higher affinity at pH 7 as compared to pH 5.5.

4. The anti-CTLA-4 antibody of claim 2, wherein the antibody binds to CTLA-4 with a higher affinity at pH 7 as compared to pH 4.5.

5. The anti-CTLA-4 antibody of any one of claims 2-4, wherein the antibody induces FcR-mediated T regulatory cell depletion in a tumor microenvironment.

6. The anti-CTLA-4 antibody of any one of claims 2-5, wherein the antibody does not confer systemic T cell activation or preferential expansion of self-reactive T cells.

7. The anti-CTLA-4 antibody of any of the preceding claims, wherein the antibody does not block binding of CTLA-4 to its B7 ligand.

8. The anti-CTLA-4 antibody of any one of the preceding claims, wherein the anti-CTLA-4 antibody has reduced affinity to soluble CTLA-4 compared to CTLA-4 located on the cell surface.

9. The anti-CTLA-4 antibody of any of the preceding claims, wherein the anti-CTLA-4 antibody is combined with an anti-PD-1 antibody or anti-PD-L1 antibody.

10. A method of identifying an anti-CTLA-4 antibody that induces lower levels of immunotherapy-related adverse events (irAE), comprising:
(a) providing cells comprising cell surface CTLA-4;
(b) contacting the cells of (b) with a candidate anti-CTLA-4 antibody;
(c) following a period of incubation, detecting the amount of cell surface CTLA-4;
(d) comparing the amount of cell surface CTLA-4 from step (c) to a threshold level, wherein the threshold level is the amount of cell surface CTLA-4 from cells that were contacted with a control anti-CTLA-4 antibody,
wherein a higher amount of cell surface CTLA-4 as compared to the threshold level identifies the candidate anti-CTLA-4 antibody as an anti-CTLA-4 antibody that induces lower levels of irAE.

11. The method of claim 10, wherein the control anti-CTLA-4 antibody is Ipilimumab or Tremelimumab.

12. The method of claim 10, wherein the cells of step (a) express human CTLA-4.

13. The method of claim 10, wherein the cell surface CTLA-4 is detectably labeled.

14. The method of claim 13, wherein the detectable label is a fluorescent tag.

15. The method of claim 14, wherein the fluorescent tag is orange fluorescent protein.

16. The method of claim 10, wherein the detecting of step (c) comprises measuring the amount of the detectable label of the cell surface CTLA-4 using a Western blot, immunohistochemistry, or flow cytometry.

17. The method of claim 10, wherein the incubation of step (c) comprises contacting the candidate anti-CTLA-4 antibody with a detectably labeled anti-IgG antibody, and measuring the amount of the detectable label of the detectably labeled anti-IgG antibody using a Western blot, immunohistochemistry or flow cytometry.

18. The method of claim 17, wherein the detectable label of the detectably labeled anti-IgG antibody comprises alex488.

19. The method of claim 10, wherein the cells are selected from the group consisting of 293T cells, Chinese Hamster Ovary cells, and T regulatory cells (Tregs).

20. An anti-CTLA-4 antibody that has higher binding affinity for CTLA-4 at a high pH of 6.5-7.5 as compared to a low pH of less than or equal to 6.

21. The antibody of claim 20, wherein the high pH is 7 and the low pH is 4.5.

22. The antibody of claim 20, wherein the high pH is 7 and the low pH is 5.5.

23. A method of screening for or designing an anti-CTLA-4 antibody for use in immunotherapy, wherein the anti-CTLA-4 antibody does not cause lysosomal CTLA-4 degradation.

24. The method of claim 23, comprising
(a) contacting the anti-CTLA-4 antibody with a CTLA-4 protein at a pH of 6.5-7.5, and quantifying the amount of anti-CTLA-4 antibody binding to the CTLA-4 protein;
(b) contacting the anti-CTLA-4 antibody with a CTLA-4 protein at a pH of 4.5-5.5, and quantifying the amount anti-CTLA-4 antibody binding to the CTLA-4 protein;
(c) comparing the amount of binding in (a) and (b),
wherein the anti-CTLA-4 antibody does not cause lysosomal CTLA-4 degradation if the amount of binding in (a) as compared to (b) is greater than or equal to a threshold level.

25. The method of claim 24, wherein the pH of (a) is 7.0, the pH of (b) is 5.5, and the threshold level is 3-fold.

26. The method of claim 24, wherein the pH of (a) is 7.0, the pH of (b) is 4.5, and the threshold level is 10-fold.

27. The method of any one of claims 24-26, wherein the amount of anti-CTLA-4 antibody binding is the amount of anti-CTLA-4 antibody required to achieve 50% maximal binding to the CTLA-4 protein.

28. The method of claim 23, wherein the anti-CTLA-4 antibody allows CTLA-4 that has been bound at a cell surface to recycle back to the cell surface after endocytosis.

29. A method of treating cancer in a subject in need thereof, comprising administering to the subject an antibody whose binding to CTLA-4 is disrupted at an acidic pH corresponding to that found in endosomes and lysosomes.

30. The method of claim 29, wherein the anti-CTLA-4 antibody exhibits a reduction of at least 3-fold in its binding to CTLA-4 at pH 5.5 as compared to pH 7.0.

31. The method of claim 29, wherein the antibody exhibits a reduction of at least 10-fold in its binding to CTLA-4 at pH 4.5 as compared to pH 7.0.

32. The method of claim 29, wherein the anti-CTLA-4 antibody exhibits a greater reduction in binding to soluble CTLA-4 than to cell-surface-bound or immobilized CTLA-4, as compared to Ipilimumab or Tremelimumab.

33. An anti-CTLA-4 antibody identified, screened or designed according to any one of claims 10-19 and 23-28.

34. A method of treating cancer in a subject in need thereof, comprising administering to the subject the anti-CTLA-4 antibody of any one of claims 1-8, 20-22, and 33.

35. The method of claim 34, wherein the anti-CTLA-4 antibody is administered in combination with an anti-PD-1 or anti-PD-L1 antibody.

36. The anti-CTLA-4 antibody of any one of claims 1-8, 20-22, and 33 for use in treating cancer in a subject.

37. The anti-CTLA-4 antibody for use of claim 36, wherein the anti-CTLA-4 antibody is administered in combination with an anti-PD-1 or anti-PD-L1 antibody.

38. Use of the antibody of any one of claims 1-8, 20-22, and 33 in the manufacture of a medicament for treating cancer.

39. The use of claim 38, wherein the anti-CTLA-4 antibody is in combination with an anti-PD-1 or anti-PD-L1 antibody.


SEQUENCE LISTING (3 sequences)

SEQ ID NO: 1 - protein, 357 aa, Artificial Sequence
  Mutant protein
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYFEGMGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 357 aa, Artificial Sequence
  Mutant protein
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYFVGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 357 aa, Artificial Sequence
  Fusion protein
IQVTQPSVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK